(19) United States
(12) Patent Application Publication
(10) Pub. No.: US 2025/0319008 A1
(43) Pub. Date: Oct. 16, 2025

(54) HAIR COMPOSITIONS USES AND METHODS THEREOF

(71) Applicant: Function Inc., New York, NY (US)

(72) Inventors: HeeSeon Kim, Palisades Park, NJ (US); Linda Foltis, Nutley, NJ (US)

(21) Appl. No.: 19/178,257

(22) Filed: Apr. 14, 2025

Related U.S. Application Data
(60) Provisional application No. 63/634,217, filed on Apr. 15, 2024.

(51) Int. Cl. A61K 8/42; A61K 8/365; A61K 8/37; A61K 8/9789; A61Q 5/02; A61Q 5/12

(52) U.S. Cl. CPC A61K 8/42; A61K 8/365; A61K 8/375; A61K 8/9789; A61Q 5/02; A61Q 5/12

(57) ABSTRACT

The application disclosed compositions that act in the hair restoration and strengthening, their uses and hair treatment methods. The active ingredients of the present composition act synergistically in increasing resistance to damages, to repair damages, as well as acting in the retention of moisture, thickening, shine, softness, thermal protection, sealing the cuticles and improving the sensorial properties of the hair.

### CROSS REFERENCE TO RELATED APPLICATION

This application claims priority to U.S. Provisional Application No. 63/634,217 filed on Apr. 15, 2024, the disclosure of which is hereby incorporated by reference in its entirety.

### TECHNICAL FIELD

The application relates to hair compositions and uses thereof. The disclosed hair compositions can be used, e.g., for restoring and strengthening hair.

### BACKGROUND

Human hair fibers are around 50-100 m in diameter, and composed of three main parts: cuticle, cortex, and medulla. The cuticle is the outer barrier protecting the cortex from external damage. The cortex makes up most of the hair mass and is responsible for the high tensile strength. While the inner part of the hair fiber is the medulla which provides a negligible contribution to its mechanical strength. Overall, the main chemical component by weight is protein composing 65-95% of the hair.

The hair cuticle accounts for 15% of the weight of a strand of hair. Cuticle cells are mechanically robust and resistant to many environmental factors, including the penetration of foreign molecules, due to the high degree of isodipeptide and disulfide bonds present in its various lamellar layers. The cortex is the middle region of the hair and represents about 80% of the weight of a strand of hair. The cortex is composed of hierarchal structures of elongated, fibrous alpha-helical keratin extending along the fiber axis embedded in an amorphous protein matrix. The medulla corresponds to a central beam of cells and is absent in some hair fibers.

The predominant proteins present in hair are alpha keratins, which act as structural building blocks of hair. Keratin proteins found in human hair contain more cysteine residues and fewer glycine residues, compared to other keratins. This higher cysteine content increases disulfide bridge formation, resulting in greater mechanical strength, and greater resistance to thermal and chemical damages. The strength of the structures formed in keratin is also related to the formation of hydrogen bonds, coulombic interactions, van der Waals forces, and hydrophobic interactions present between different amino acid residues.

Hair suffers damage from many different sources. Hair can be damaged in a variety of ways including environmental, chemical, over washing, or thermal damage. The hair fiber can be damaged by environmental factors such as exposure to UV and chlorine; chemical factors such as dyeing, bleaching, perming, and over-frequent washing with harsh surfactant-based cleansing shampoo compositions; and mechanical factors such as prolonged use of heated styling appliances. Consequently, proteins in hair gradually disappear, the hair dries and becomes brittle, forms split ends, and loses its strength. To make matters worse, with the loss of the proteins, the hair becomes thinner and the likelihood of damage increases. The hair, once damaged, is unable to restore itself to its original state.

Therefore, there is a need for improved compositions and methods to protect the hair from damage, and in case of damage, to repair the damaged hair, to keep the hair beautiful and healthy.

### BRIEF SUMMARY

The application addresses the need by providing a hair composition with ingredients that act synergistically to repair hair cortex, protect hair cuticle, and maintain hair integrity. The hair compositions can be used in hair products such as a shampoo, conditioner, mask, leave-in treatment product.

Accordingly, in one general aspect, the application relates to a hair composition comprising:
(1) a first component comprising at least one of a compound of Formula (I) and a compound of Formula (II)

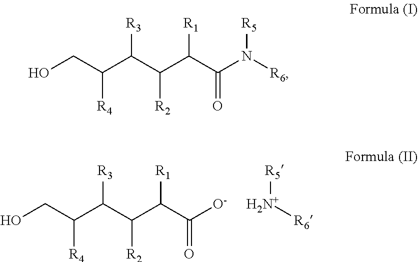

wherein R₁-R₄ and R₁′-R₄′ are independently hydrogen, a hydrocarbon radical having 1 to 10 carbon atoms, a hydroxyl group, an amino group, a sulfhydryl group, an aryl group, or a halogen, and
R₅, R₆, R₅′ and R₆′ are independently hydrogen, an aliphatic hydrocarbon group, an alicyclic hydrocarbon group, an aryl group, an alkylaryl group or a heterocyclic group, with the proviso that R₅′ and R₆′ are not simultaneously hydrogen, wherein the aliphatic hydrocarbon group, the alicyclic hydrocarbon group, the aryl group, the alkylaryl group or the heterocyclic group is substituted with at least one hydroxyl group; and
(2) a second component comprising:
   a. an α,β unsaturated dicarboxylic acid and/or an ester thereof;
   b. a nonionic crosslinking agent selected from the group consisting of polyols, saccharides or a combination thereof; and
   c. an ionic cross-linking agent selected from the group consisting of hydrolyzed proteins, amino acids or a combination thereof.

In certain embodiments, a hair composition of the application comprises the first component comprising the compound of Formula (I) and the compound of Formula (II), and the second component that further comprises one or more plant or seed extracts. Preferably, the total amount of the first component is about 0.2% to 2.5% by weight based on the total weight of the hair composition, and the total amount of the second component is about 0.05% to 2% by weight based on the total weight of the hair composition.

In certain embodiments, a hair composition of the application further comprises one or more additional components. For example, the hair composition can further comprise one or more components selected from the group consisting of surfactants, fatty acid soap, hair and skin conditioning agents, suspending aids, emollients, emulsifiers, rheology modifiers, thickening agents, vitamins, hair growth promoters, self-tanning agents, sunscreens, skin lighteners, anti-aging compounds, anti-wrinkle compounds, hair coloring treatment agents, hair color protecting agents, hair styling products, anti-cellulite compounds, anti-acne compounds, anti-dandruff agents, anti-inflammatory compounds, analgesics, antiperspirant agents, deodorant agents, hair fixatives, particulates, abrasives, moisturizers, antioxidants, keratolytic agents, anti-static agents, foam boosters, hydrotropes, solubilizing agents, chelating agents, antimicrobial agents, antifungal agents, pH adjusting agents, chelating agents, buffering agents, botanicals, hair colorants, oxidizing agents, reducing agents, hair and skin bleaching agents, pigments, anticaries, anti-tartar agents, anti-plaque agents, solvents, and combinations thereof.

In certain embodiments, a hair composition of the application is a rinse-off composition, such as a shampoo, a hair conditioner, or a hair mask. In certain embodiments, a hair composition of the application is a leave-on composition, such as a hair serum or a hair styling product.

In certain embodiment, a hair composition of the application is formulated for the treatment of any hair types, such as straight hairs, wavy hairs, curly hairs, coily hairs, or a combination thereof.

In another general aspect, the application relates to a method of preparing a hair composition of the application, comprising combining the components to thereby obtain the hair composition.

Other aspects of the application relate to uses of a hair composition of the application for hair treatments. In one embodiment, the application relates to a method for treating hair comprising applying to the hair a hair composition of the application. In another embodiment, the application relates to a method of strengthening hair fibers comprising applying to the hair fibers a hair composition of the application. In yet another embodiment, the application relates to a method of reducing and/or preventing hair breakage comprising applying to the hair a hair composition of the application. The hair compositions can be applied to any hair or hair fibers in need of the treatment, such as virgin hair, hair treated with a chemical or mechanical hair treatment (e.g., bleached hairs, dyed hairs), and damaged or weakened hair fibers.

### BRIEF DESCRIPTION OF THE DRAWING

The foregoing and other objects, aspects, features, and advantages of exemplary embodiments will become more apparent and may be better understood by referring to the following description taken in conjunction with the accompanying drawing.

FIG. 1 illustrates the comparison of hair breakage performance over time with different hair compositions.

### DETAILED DESCRIPTION

The application discloses hair compositions that act in the hair restoration and strengthening, their preparation methods and uses thereof in hair treatment.

Surprisingly, a synergistic effect was observed between two components in a hair composition of the application: a first component comprising gluconamides and alkyl ammonium gluconate salts, and a second component comprising bifunctional α,β-unsaturated compounds, and ionic and non-ionic crosslinking agents. The active ingredients of the hair composition act synergistically in increasing resistance to damages, repairing damages, as well as in the retention of moisture, thickening, shine, softness, thermal protection, sealing the cuticles and improving the sensorial properties of the hair.

While not wishing to be bound by theory, it is believed that the disclosed hair compositions can repair the cortex through crosslinking the broken bonds, creating new hydrogen, covalent bonds and ionic bonds, and promoting the formation of crosslinks among hair protein. The hair composition can increase the strength of the hair, through reactions with an amino group, or a sulfhydryl or thiol groups (—SH) contained in the side chains of amino acids present in the keratin structure with derivatives of α,β-unsaturated compounds. These Michael and Aza-Michael reactions lead to the formation of tertiary amines, and, in some embodiments, restoring disulfide bonds. In particular, itaconic acid in the hair composition acts as a Michael acceptor, reacting covalently with both the amino groups (—NH₂) and the sulfhydryl or thiol groups (—SH) of the keratin of the hair through Aza-Michael and Michael reactions, which can form modified keratin. Pro-vitamin B5 and the polysaccharides extracted from linseed and chia seeds present in the hair composition promote moisture retention, thickening, shine, softness, thermal protection, sealing the cuticles and enhance the look and feel of the hair. The crosslinking agents, such as arginine, low molecular weight hydrolyzed proteins and the different amino acid residues present in the protein structure of the hair. The inclusion of non-ionic cross-linking agents, such as disaccharides, pro-vitamin B5 and polysaccharides in a hair composition can form crosslinks between the keratin polypeptide chains. The inclusion of gluconamides and corresponding alkyl ammonium gluconate in the hair composition, surprisingly, demonstrated a significant synergistic association between the keratin polypeptide chains, greatly increased the hair's strength.

In some embodiments, a hair composition of the application help form a high-density cross-linking process. In some embodiments, the hair compositions promote the action of arginine through intermolecular interactions with itaconic acid. In some embodiments, the hair compositions promote crosslinking process through ion-dipole, hydrogen bonds, dipole-dipole, or the ion-dipole interactions.

In some embodiments, the hair compositions can promote hair strength by different mechanisms of action, since methionine sulfoxide catalyzes the formation of disulfide bonds and, in parallel, the bifunctional α,β-unsaturated compounds enable the formation of two covalent Aza-Michael bonds, leading to the modification of keratin polypeptide chains. In addition, the formation of crosslinks can occur through amino acids or hydrolyzed proteins, through intermolecular interactions, forming “bridges” or “ionic crosslinks” with these modified polypeptide chains, promoting surprisingly increases in the maximum rupture tension of bleached hair. The increase in strength can be potentiated by associating with pro-vitamin B5, saccharides and polysaccharides. Furthermore, the hair strength can be increased significantly by including gluconamides and corresponding alkyl ammonium gluconates in the hair composition. This can be attributed to the synergistic effects of all the ingredients.

In some embodiments, the hair compositions can increase hair strength. In some embodiments, the hair compositions can protect the hair from damage. In some embodiments, the hair compositions can repair hair damage, in case of damage. In some embodiments, the hair compositions reduce hair damage and increase survival probability of hair fiber.

In some embodiments, the hair compositions can proactively treat chemically damaged hair. In some embodiments, the hair compositions increase hair's denaturation enthalpies. In some embodiments, the hair compositions increase hair's denaturation enthalpies during bleaching, therefore increase hair resistance to damages during bleaching. In some embodiments, the hair compositions increase hair's denaturation enthalpies after bleaching. In some embodiments, the hair compositions can help to increase the cycles take to break the hair fiber.

In some embodiments, the hair compositions can form a protective barrier on the hair, with no build-up effect. In some embodiments, the hair compositions can protect cuticle integrity. In some embodiments, the hair compositions can increase hair shininess. In some embodiments, the hair compositions can improve hair combability. In some embodiments, the hair compositions can provide hair protection and repair against chemical procedures. In some embodiments, the hair compositions can form smart bonding to avoid brittle effect.

In some embodiments, the hair compositions can protect hair from chemical aggressions such as bleaching and straightening. In some embodiments, the hair compositions can protect hair in different hair types. In some embodiments, the hair compositions can protect the hair from damage, such as coloring, without affecting the dyed colors. In some embodiments, the hair compositions don't affect bleaching efficacy. In some embodiments, the hair compositions don't affect the color parameters of colored hair when hair compositions are added to hair coloration. In some embodiments, the hair compositions are used in bleach and color products.

In some embodiments, the hair compositions reduce dry friction. In some embodiments, the hair compositions reduce dry friction in hair coloration process. In some embodiments, the hair compositions improve dry combability. In some embodiments, the hair compositions reduce hair combing efforts. In some embodiments, the hair compositions protect hair against oxidative damage. In some embodiments, the hair compositions improve the resilience of the hair. In some embodiments, the hair compositions' beneficial effects are maintained even after ceasing use.

In some embodiments, the hair compositions promote moisture retention, thickening, shine, softness, or thermal protection. In some embodiments, the hair compositions seal the cuticles, and increase the look and feel of the hair. In some embodiments, the hair compositions penetrate the hair fiber. In some embodiments, the hair compositions enhance fiber alignment. In some embodiments, the hair compositions penetrate the cortex.

In some embodiments, the hair compositions are based on natural glucose-based chemistry. In some embodiments, the hair compositions are biodegradable. In some embodiments, the hair compositions work across a wide range of PH range.

In some embodiments, the hair compositions are used in conditioners, leave-in, masks, serum, creams, shampoos, sprays, lotions, bleaching and coloring products and gels.

### Definitions

The disclosed compositions and methods may be understood more readily by reference to the following detailed description, which forms a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Various publications, articles and patents are cited or described in the background and throughout the specification, each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the term “about” preceding a numerical value or a series of numerical values means ±10% of the numerical value unless otherwise indicated. For example, “about 100 mg” means 90 to 110 mg.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “one or more” when referring to an optional substituent means that the subject group is optionally substituted with one to twenty-five substituents. In some embodiments, the subject group is optionally substituted with one to ten substituents. In some embodiments, the subject group is optionally substituted with eleven to twenty substituents. In some embodiments, the subject group is optionally substituted with twenty to twenty-five substituents. In some embodiments, the subject group is optionally substituted with one to five substituents. In some embodiments, the subject group is optionally substituted with five to ten substituents. In some embodiments, the subject group is optionally substituted with ten to fifteen substituents. In some embodiments, the subject group is optionally substituted with sixteen to twenty substituents. In some embodiments, the subject group is optionally substituted with one substituent. In some embodiments, the subject group is optionally substituted with two substituents. In some embodiments, the subject group is optionally substituted with three substituents. In some embodiments, the subject group is optionally substituted with four substituents. In some embodiments, the subject group is optionally substituted with five substituents. In some embodiments, the subject group is optionally substituted with six substituents. In some embodiments, the subject group is optionally substituted with seven substituents. In some embodiments, the subject group is optionally substituted with eight substituents. In some embodiments, the subject group is optionally substituted with nine substituents. In some embodiments, the subject group is optionally substituted with ten substituents.

As used herein the term “hair” or “hair fiber(s)” to be treated can be “living,” e.g., on a living body or can be “non-living,” e.g., in a wig, hairpiece or other aggregation of nonliving keratinous fibers. Mammalian, human hair, wool, fur and other keratin containing fibers are suitable substrates for compositions according to embodiments of the invention. “Hair” and “hair fiber(s) are used interchangeably in the application.

As used herein, “virgin hair” refers to hair that has never been treated chemically and/or mechanically by a hair treatment. Examples of the hair treatment include but are not limited to coloring, bleaching, relaxing, straightening, perming, grooming, etc.

As used herein, the expression “leave-on composition” or “leave-in composition” refers to a hair composition that is not rinsed off from the hair shortly after the composition is applied to the hair. A “leave-on composition” can be a hair composition formulated for any leave-in treatment of hair, including but not limited to, hair serum and hair styling products.

As used herein, the expression “rinse-off composition” designates a hair composition that is rinsed off from the hair with water shortly after the composition is applied to the hair. A “rinse-off composition” can be a hair composition formulated for any rinse-off treatment of hair, including but not limited to, shampoo, hair conditioner and hair mask.

As used herein, the expression “dyed hair” means hair which has been colored with a permanent, semi-permanent or temporary color, which can be the same or different from the original color of the hair.

The hair fibers can include virgin hair, hair treated with a chemical or mechanical hair treatment, and damaged or weakened hair fibers.

As used herein, the term “crosslinks” refers to cross-links between keratin polypeptide chains, promoting an increase in deformation resistance in the stretching of capillary structures.

As used herein, the term “ionic cross-linking agent” refers to ionically charged molecules capable of performing crosslinks between keratin polypeptide chains.

As used herein, the term “non-ionic cross-linking agents” refers to uncharged molecules capable of increasing crosslinks between keratin polypeptide chains, promoting a synergistic association in increasing mechanical strength of the hair fiber.

As used herein, the term “bifunctional compound” refers to a compound having at least two organic functions. Examples of “bifunctional compound” include, for example, a compound having two carboxylic groups, an amide and a carboxylic group, etc. Non-exhaustive examples of bifunctional compounds can be found in US2020315936, the relevant content of which is herein incorporated by reference in its entirety.

Non-limiting examples of saccharides include sugars, starches and celluloses.

It is understood in the application that the term “derivatives” in connection with a compound comprises the compound's organic derivatives, hydrates, stereoisomers, multimers, cyclic forms, drug-conjugates, prodrugs and their cosmetically or pharmaceutically acceptable salts. For example, “derivatives” of an α,β-unsaturated compound comprise organic derivatives, such as esters, amides, hydroxyls, ethers, amines, among other common organic functions of the α,β-unsaturated compound.

It is understood in the application that unless specified otherwise, the “%” refers to the “% by weight”.

As used herein, the term “cosmetically acceptable” refers to those compounds, materials, components, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “cosmetically or dermatologically acceptable” refers to the compositions or components described are suitable for use in contact with human skin tissue without undue toxicity, incompatibility, instability, allergic response, and the like.

The hair composition or formulation is cosmetically or pharmaceutically acceptable or suitable for human and/or veterinary use. An acceptable or suitable cosmetic and pharmaceutical composition or formulation is understood to mean any type of solvent, dispersion medium, encapsulation, permeation agents, surfactants, preservatives and other ingredients, alone or in combination, and which are physiologically compatible, do not present instability, incompatibility, toxicity and do not cause allergic reactions or irritability. Examples of cosmetically acceptable ingredients and/or excipients can be found in handbooks and pharmacopoeias for each country. Non-exhaustive examples of pharmacopeia are listed in the American Pharmacopeia (“United States Pharmacopeia”), the European Pharmacopeia and the International Pharmacopeia.

The hydroxyl group means a “—OH” group.

The term “amino”, as used herein, is intended to include the —NH₂ radical, and any substitutions of the N atom.

The sulfhydryl group means a “—SH” group.

The terms “halogen” and “halo”, as used herein, represent chlorine, fluorine, bromine or iodine.

“Alkyl” refers to an optionally substituted straight-chain, or optionally substituted branched-chain saturated hydrocarbon monoradical having from one to about twelve carbon atoms, or from one to ten carbon atoms.

The hydrocarbon group is an organic compound consisting entirely of hydrogen and carbon. It can be saturated or unsaturated, linear, or branched, substituted or unsubstituted.

The aliphatic hydrocarbon group used herein can include saturated or unsaturated, liner or branched, substituted or unsubstituted aliphatic hydrocarbon groups. Examples of the aliphatic hydrocarbon groups can include, but are not limited to, a straight or branched alkyl group having 1 to about 12 carbon atoms, such as a methyl group, an ethyl group, a propyl group, an isopropyl group, a butyl group, an isobutyl group, an sec-butyl group, a t-butyl group, a pentyl group, a hexyl group, a heptenyl group, an octyl group, and a decyl group; an alkenyl group having 1 to 12 carbon atoms, such as a vinyl group, an allyl group, a 1-propenyl group, an isopropenyl group, and a 2-butenyl group; and an alkynyl group having 1 to 12 carbon atoms, such as a 2-propynyl group, and a 2-butynyl group.

The alicyclic hydrocarbon group used herein can include saturated or unsaturated, substituted or unsubstituted alicyclic hydrocarbon groups. Examples of the alicyclic groups can include, but are not limited to, a cycloalkyl group having about 3 to about 10 carbon atoms, such as a cyclobutyl group, a cyclopentyl group, a cyclohexyl group, a cyclooctyl group; and a cycloalkenyl group having about 3 to about 10 carbon atoms, such as a cyclopentenyl group, and a cyclohexenyl group.

“Aryl” refers to a radical derived from a hydrocarbon ring system comprising hydrogen, 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. It can comprise about 6 to about 14 carbon atoms, such as a phenyl group, or a naphthyl group.

“Alkenyl” refers to an optionally substituted straight-chain, or optionally substituted branched-chain hydrocarbon monoradical having one or more carbon-carbon double-bonds. The group may be in either the cis or trans conformation about the double bond(s) and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated.

The term “alkylaryl” as used herein, refers to an alkyl group substituted with an aryl group.

The heterocyclic group used herein can include those containing at least one heteroatom selected from the group consisting of a nitrogen atom, an oxygen atom, and a sulfur atom. The heterocyclic group may be an aromatic heterocyclic group, a non-aromatic heterocyclic group, or a compound heterocyclic group.

A heterocyclic ring of the heterocyclic group can include a nitrogen-containing heterocyclic ring such as pyrroline, pyrrole, piperidine, piperazine, pyridine, pyrimidine, pyridazine, triazole, and quinoline; an oxygen-containing heterocyclic ring such as tetrahydrofuran, furan, and pyran; a sulfur-containing heterocyclic ring such tetrahydrothiophene, and thiophene; and a heterocyclic ring containing at least two heteroatoms selected from the group consisting of a nitrogen atom, an oxygen atom, and a sulfur atom, such as thiazoline, thiazolidine, thiazoie, thiazine, and morpholine.

An “amino acid” refers to a natural amino acid or a non-natural amino acid. The natural amino acid is a naturally-occurring amino acid, including, but not limited to, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan.

A polyol is an organic compound containing multiple hydroxyl groups (—OH). Non-limiting examples, polyols containing two, three and four hydroxyl groups are diols, triols, and tetrols, respectively.

The term “saccharides” or “carbohydrates”, refers to polyhydroxylated aldehydes or ketones with the empirical chemical formula Cₙ(H₂O)ₘ where n and m may be different. The saccharides are divided into four chemical groups: monosaccharides, disaccharides, oligosaccharides, and polysaccharides.

### Hair Compositions

In some embodiments, a hair composition of the application comprises:
(1) a first component comprising at least one of a compound of Formula (I) and compound of Formula (II):

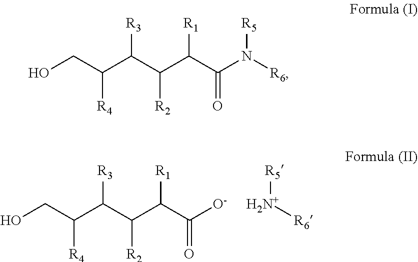

wherein R₁-R₄ and R₁′-R₄′ are independently hydrogen, a hydrocarbon radical having 1 to 10 carbon atoms, a hydroxyl group, an amino group, a sulfhydryl group, an aryl group, or a halogen, and
R₅, R₆, R₅′ and R₆′ are independently hydrogen, an aliphatic hydrocarbon group, an alicyciic hydrocarbon group, an aryl group, an alkylaryl group or a heterocyclic group, with the proviso that R₅′ and R₆′ are not simultaneously hydrogen, wherein the aliphatic hydrocarbon group, the alicyclic hydrocarbon group, the aryl group, the alkylaryl group or the heterocyclic group is substituted with at least one hydroxyl group; and
(2) a second component comprising bifunctional α,β-unsaturated compounds in combination with ionic and non-ionic cross-linking agents, preferably the second component comprises:
   a. an α,β unsaturated dicarboxylic acid and/or an ester thereof;
   b. a nonionic crosslinking agent selected from the group consisting of polyols, saccharides or a combination thereof; and
   c. an ionic cross-linking agent selected from the group consisting of hydrolyzed proteins, amino acids or a combination thereof.

In some embodiments, for the compound of Formula (I), R₁-R₄ are each a hydroxyl group, R₅ and R₆, are independently hydrogen, or a —CH₂CH₂CH₂OH group; preferably R₁-R₄ are each a hydroxyl group, R₅ is a hydrogen, and R₆ is a —CH₂CH₂CH₂OH group.

In some embodiments, the compound of Formula (I) is a compound selected from gluconamides. For example, the compound having the chemical structure of Formula (I) is a monoamide or a bisamides. Preferably, the compound of Formula (I) is hydroxypropylgluconamide having the following structure:

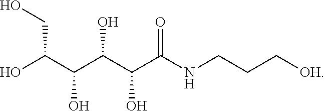 [SMILES: O=C(NCCCO)[C@H](O)[C@@H](O)[C@H](O)[C@H](O)CO]

In some embodiments, for the compound of Formula (II), R₁′-R₄′ are each a hydroxyl group, R₅′ and R₆′ are independently hydrogen, or a —CH₂CH₂CH₂OH group; preferably R₁′-R₄′ are each a hydroxyl group, R₅′ is a hydrogen, R₆′ is a —CH₂CH₂CH₂OH group. In some embodiments, the compound of Formula (II) is hydroxypropylammonium gluconate having the following structure:

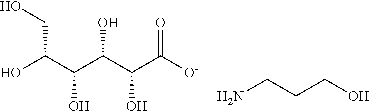 [SMILES: O=C([O-])[C@H](O)[C@@H](O)[C@H](O)[C@H](O)CO ; NCCCO]

In some embodiments, the first component comprises the compound of formula (I) and the compound of formula (II).

In some embodiments, the amounts of the compound of Formula (I) and the compound of Formula (II) can be varied in the hair composition. The mole percentages of the compound of Formula (I) to the compound of Formula (II) can vary from 0.1 mole % to 99.9 mole %. In some embodiments, the molar ratio of the compound of Formula (I) to the compound of Formula (II) can be 1:99 to 99:1. In some embodiments, the molar ratio of the compound of Formula (I) to the compound of Formula (II) can be 20:80 to 80:20. In some embodiments, the molar ratio of the compound of Formula (I) to the compound of Formula (II) can be 30:70 to 70:30. In some embodiments, the molar ratio of the compound of Formula (I) to the compound of Formula (II) can be 40:60 to 60:40. In some embodiments, the molar ratio of the compound of Formula (I) to the compound of Formula (II) can be 45:55 to 55:45.

In certain embodiments, the first component comprises one or more additional ingredients suitable for hair composition. For example, the first component can comprise water, preservatives and/or pH adjuster. In certain embodiments, the first component comprises hydroxypropylgluconamide, hydroxypropylammonium gluconate, water, preservatives and pH adjuster. Preferably, the first component comprises 50% (w/w) hydroxypropylgluconamide and hydroxypropylammonium gluconate, 49% (w/w) water, and 1% (w/w) preservatives (such as benzyl alcohol, sodium benzoate, potassium sorbate) and pH adjuster (such as tartaric acid), wherein the weight percentage (% (w/w)) is relative to the total weight of the first component. More preferably, the first component is FiberHance™ bm, commercially available from Ashland.

The compounds of formula (I) and formula (II) can be made using methods known in the art in view of the present disclosure. A first component comprising compounds of formula (I) and formula (II) and optionally other ingredients can be prepared by combining the compounds of formula (I) and the compound of formula (II), and optionally the other ingredients, using methods known in the art in view of the present disclosure. See, e.g., U.S. Ser. No. 10/568,823, the relevant content of which is incorporated herein by reference in its entirety.

In some embodiments, the second component comprises bifunctional α,β-unsaturated compounds in combination with ionic and/or non-ionic cross-linking agents.

In some embodiments, the α,β-unsaturated compound is an α,β-unsaturated dicarboxylic acid or ester. In some embodiments, the α,β-unsaturated compound comprises itaconic acid, acrylic acid, maleic acid, fumaric acid, methacrylic acid and/or maleic anhydride, or derivatives thereof. In some embodiments, the α,β-unsaturated compound is a polyol diester. In some embodiments, the α,β-unsaturated compound is panthenol di-itaconate. In some embodiments, the α,β-unsaturated compound is obtained from a fermented Aspergillus extract. In some embodiments, the α,β-unsaturated compound comprises itaconic acid.

In some embodiments, the nonionic crosslinking agents comprise polyols, saccharides or a combination thereof. In some embodiments, the nonionic crosslinking agents comprise panthenol. In some embodiments, the nonionic crosslinking agents comprise propanediol. In some embodiments, the nonionic crosslinking agents comprise 1,3-propanediol.

In some embodiments, the saccharides are polysaccharides and/or disaccharides. In some embodiments, the saccharides are of plant origin. In some embodiments, the saccharide is trehalose. In some embodiments, the saccharides are obtained from a plant or seed extracts, such as a flaxseed or chia extract, a Linum usitatissimum seed extract, or Salvia hispanica seed extract. In some embodiments, linseed is the species and cultivars of Linum spp. In some embodiments, chia is the species and cultivars of Salvia spp. In some embodiments, the saccharides are a combination of chia extract and Linseed seed extract.

In some embodiments, the ionic cross-linking agents comprise hydrolyzed proteins, amino acids or a combination thereof. In some embodiments, the ionic cross-linking agents comprise amino acids. In some embodiments, the ionic cross-linking agents comprise natural amino acids. In some embodiments, the amino acid is selected from arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. In some embodiments, the ionic cross-linking agents comprise arginine. In some embodiments, the amino acid comprises cysteine.

In some embodiments, the second component comprises one or more a, p unsaturated dicarboxylic acid and/or an ester thereof; one or more nonionic crosslinking agents selected from the group consisting of polyols, saccharides or a combination thereof; and one or more ionic cross-linking agents selected from the group consisting of hydrolyzed proteins, amino acids or a combination thereof.

In some embodiments, the second component comprises one or more bifunctional α,β-unsaturated compound selected from the group consisting of itaconic acid and its derivatives; one or more crosslinking agents including a) one or more non-ionic crosslinking agents selected from the group consisting of polyols, saccharides or a combination thereof; and b) arginine.

In some embodiments, the second component comprises itaconic acid, arginine, panthenol, or a combination thereof. In some embodiments, the second component comprises itaconic acid, arginine, panthenol, Linum usitatissimum seed extract, Salvia hispanica seed extract, or a combination thereof. In some embodiments, the second component comprises itaconic acid, arginine, panthenol, Linum usitatissimum seed extract, Salvia hispanica seed extract, polyglyceryl-10 laurate, or a combination thereof. In some embodiments, the second component comprises itaconic acid, arginine, panthenol, Linum usitatissimum seed extract, Salvia hispanica seed extract, polyglyceryl-10 laurate, xylitol or a combination thereof. In some embodiments, the second component comprises itaconic acid, arginine, panthenol, Linum usitatissimum seed extract, Salvia hispanica seed extract, polyglyceryl-10 laurate, xylitol, caprylic acid or a combination thereof.

In some embodiments, the second component comprises dicarboxylic acid, amino acid, provitamin B5 and polysaccharides extracted from seeds of Flaxseed (Linum usitatissimum) and Chia (Salvia hispanica). Preferably, the second component comprises 30-70% (w/w) water, 5-20% (w/w) itaconic acid, 5-20% (w/w) arginine, 1-15% (w/w) panthenol, 1-10% (w/w) Linum usitatissimum (Linseed) seed extract, 1-9% (w/w) Salvia hispanica seed extract, 1-8% (w/w) polyglyceryl-10 Laurate, 0.2-0.9% (w/w) xylitol, and 0.2-0.9% (w/w) caprylic acid, wherein the weight percentage is relative to the total weight of the second component. More preferably the second component is Restart Pro, commercially available from Chemyunion.

A second component useful for a hair composition of the application can be prepared using methods known in the art in view of the present disclosure. See, e.g., WO2022/217330, the relevant content of which is incorporated herein by reference.

In some embodiments, a hair composition of the application comprises: a first component comprising hydroxypropylgluconamide, hydroxypropylammonium gluconate, or a combination thereof; and a second component comprising itaconic acid, arginine, panthenol, or a combination thereof.

In some embodiments, a hair composition of the application comprises: a first component comprising hydroxypropylgluconamide, hydroxypropylammonium gluconate, or a combination thereof; and a second component comprising itaconic acid, arginine, panthenol, Linum usitatissimum seed extract, Salvia hispanica seed extract, or a combination thereof.

In some embodiments, a hair composition of the application comprises: a first component comprising hydroxypropylgluconamide, hydroxypropylammonium gluconate, or a combination thereof; and a second component comprising itaconic acid, arginine, panthenol, Linum usitatissimum seed extract, Salvia hispanica seed extract, polyglyceryl-10 laurate, or a combination thereof.

In some embodiments, a hair composition of the application comprises: a first component comprising 50% (w/w) hydroxypropylgluconamide and hydroxypropylammonium gluconate, 49% (w/w) water, and 1% (w/w) preservatives (such as benzyl alcohol, sodium benzoate, potassium sorbate) and pH adjuster (such as tartaric acid), wherein the weight percentage is relative to the total weight of the first component, and a second component comprising 30-70% (w/w) water, 5-20% (w/w) itaconic acid, 5-20% (w/w) arginine, 1-15% (w/w) panthenol, 1-10% (w/w) Linum usitatissimum (Linseed) seed extract, 1-9% (w/w) Salvia hispanica seed extract, 1-8% (w/w) polyglyceryl-10 Laurate, 0.2-0.9% (w/w) xylitol, and 0.2-0.9% (w/w) caprylic acid, wherein the weight percentage is relative to the total weight of the second component.

In some embodiments, a hair composition of the application comprises a first component comprising FiberHance bm and a second component comprising Restart Pro.

According to embodiments of the application, a hair composition of the application comprises 0.1-5% (w/w) of the first component and 0.1-5% (w/w) of the second component, and one or more additional ingredients suitable for hair compositions. In some embodiments, the hair composition comprises about 0.1%, 0.5%, 1%, 1.5%, 2%, 2.5%, 3%, 3.5%, 4%, 4.5%, 5% (w/w) or any value in between of the first component. In some embodiments, the hair composition comprises about 0.1%, 0.5%, 1%, 1.5%, 2%, 2.5%, 3%, 3.5%, 4%, 4.5%, 5% (w/w) or any value in between of the second component. In some embodiments, the hair composition comprises 0.1-5% (w/w), such as 0.1%, 0.5%, 1%. 1.5%, 2%, 15%, 3%, 3.5%, 4%, 4.5%. 5% (w/w) or any value in between of the first component, and 0.1-5% (w/w), such as 0.1%, 0.5%. 1%, 1.5%, 2%, 2.5%, 3%, 3.5%, 4%, 4.5%, 5% (w/w) or any value in between of the second component.

In some embodiments, the hair composition comprises 0.1-3%, such as 0%, 0.3%, 0.6%, 0.9%, 1.2%, 1.5%, 1.8%, 2.1%, 2.4%, 2.7%, 3% (w/w) or any value in between of the first component. In sone embodiments, the hair composition comprises 0.1-3%, such as 0.1%, 0.3%, 0.6%, 0.9%, 1.2%. 1.5%, 1.8%, 2.1%, 2.4%, 2.7%, 3% (w/w) or any value in between of the second component. In some embodiments, the hair composition comprises 0.1-3%, such as 0.1%, 0.3%, 0.6%, 0.9%, 1.2%, 1.5%, 1.8%. 2.1%, 2.4%, 2.7%, 3% (w/w) or any value in between of the first component, and 0.1-3%, such as 0.1%, 0.3%, 0.6%, 0.9%, 1.2%, 1.5%, 1.8%, 2.1%, 2.4%, 2.7% 3% (w/w) or any value in between of the second component.

In some embodiments, the hair composition comprises 0.75-2.5%, such as 0.75%, 1%, 1.25%, 1.5%, 1.75%, 2%, 2.25%, 2.5% (w/w) or any value in between of the first component. In some embodiments, the hair composition comprises 0.2-2%, such as 0.2%, 0.4%, 0.6%, 0.8%, 1.0%, 1.2%, 1.4%, 1.6%, 1.8%, 2% (w/w) or any value in between of the second component. In some embodiments, the hair composition comprises 0.75-2.5%, such as 0.75%. 1%, 1.25%, 1.5%, 1.75%, 2%. 2.25%, 2.5% (w/w) or any value in between of the first component, and 0.2-2%, such as 0.2%, 0.4%, 0.6%, 0.8%, 1.0%, 1.2%. 1.4%, 1.6%, 1.8%, 2% (w/w) or any value in between of the second component.

In some embodiments, the hair composition comprises 0.75-2.5%, such as 0.75%, 1%, 1.25%, 1.5%, 1.75%, 2%, 2.25%, 2.5% (w/w) or any value in between of the first component. In some embodiments, the hair composition comprises 0.2-2%, such as 0.2%, 0.4%, 0.6%, 0.8%, 1.0%, 1.2%, 1.4%, 1.6%. 1.8%, 2% (w/w) or any value in between of the second component. In some embodiments, the hair composition comprises 0.75-2.5%, such as 0.75%, 1%. 1.25%, 1.5%, 1.75%, 2%, 2.25%, 2.5% (w/w) or any value in between of the first component, and 0.2-2%, such as 0.2%, 0.4%, 0.6%, 0.8%, 1.0%, 1.2%, 1.4%, 1.6%, 1.8%, 2% (w/w) or any value in between of the second component.

In some embodiments, the hair composition comprises 1-2%, such as 1%, 1.2%, 1.4%, 1.6%, 1.8%, 2% (w/w) or any value in between of the first component. In some embodiments, the hair composition comprises 0.2-1%, such as 0.2%, 0.4%, 0.6%, 0.8%, 1.0% (w/w) or any value in between of the second component. In some embodiments, the hair composition comprises 0.5-1.5%, such as 0.5%, 0.75%, 1.0%, 1.25%, 1.5% (w/w) or any value in between of the second component. In some embodiments, the hair composition comprises 1-2%, such as 1%, 1.2%, 1.4%, 1.6%, 1.8%, 2% (w/w) or any value in between of the first component, and 0.2-1%, such as 0.2%, 0.4%, 0.6%, 0.8%, 1.0% (w/w) or any value in between of the second component. In some embodiments, the hair composition comprises 1-2%, such as 1%, 1.2%, 1.4%, 1.6%, 1.8%, 2% (w/w) or any value in between of the first component, and 0.5-1.5%, such as 0.5%, 0.75%, 1.0%, 1.25%, 1.5% (w/w) or any value in between of the second component.

In some embodiments, the hair composition comprises: (1) 1% (w/w) of the first component and 0.2% (w/w) of the second component; (2) 2% (w/w) of the first component and 0.35% (w/w) of the second component; (3) 2% (w/w) of the first component and 0.5% (w/w) of the second component; (4) 1% (w/w) of the first component and 0.5% (w/w) of the second component; (5) 2% (w/w) of the first component and 1.5% (w/w) of the second component; and (6) 2% (w/w) of the first component and 1% (w/w) of the second component.

In some embodiments, the hair composition comprises: (1) 2% (w/w) of the first component and 2% (w/w) of the second component; (2) 1% (w/w) of the first component and 0.35% (w/w) of the second component; (3) 1% (w/w) of the first component and 0.3% (w/w) of the second component; (4) 1% (w/w) of the first component and 0.35% (w/w) of the second component.

Hair compositions according to embodiments of the application include leave-on compositions and rinse-off compositions. Examples of such hair compositions include, but are not limited to hair serum, hair styling products, shampoos, hair conditioners, hair masks, etc.

Hair compositions according to embodiments of the application can be formulated for application to any type of hairs, such as straight, wavy, curly, and/or coily hairs.

In some embodiments, a hair composition of the application further comprises at least one additional ingredient, such as one selected from the group consisting of surfactants, fatty acid soap, hair and skin conditioning agents, suspending aids, emollients, emulsifiers, rheology modifiers, thickening agents, vitamins, hair growth promoters, self-tanning agents, sunscreens, skin lighteners, anti-aging compounds, anti-wrinkle compounds, hair coloring treatment agents, hair color protecting agents, hair styling products, anti-cellulite compounds, anti-acne compounds, anti-dandruff agents, anti-inflammatory compounds, analgesics, antiperspirant agents, deodorant agents, hair fixatives, particulates, abrasives, moisturizers, antioxidants, keratolytic agents, anti-static agents, foam boosters, hydrotropes, solubilizing agents, chelating agents, antimicrobial agents, antifungal agents, pH adjusting agents, chelating agents, buffering agents, botanicals, hair colorants, oxidizing agents, reducing agents, hair and skin bleaching agents, pigments, anticaries, anti-tartar agents, anti-plaque agents, solvents, and combinations thereof.

In some embodiments, a hair composition of the application further comprises a preservative. In some embodiments, the preservative comprises a benzoic acid or salt thereof, xylityl sesquicaprylate, sodium benzoate, potassium sorbate, benzyl alcohol, or ethylhexylglycerin.

In some embodiments, a hair composition of the application further comprises surfactants. In some embodiments, the surfactants comprise cationic surfactant, non-ionic surfactant, amphoteric surfactant, anionic surfactant, sodium benzoate, potassium sorbate, or a combination thereof.

In some embodiments, a hair composition of the application further comprises one or more cationic surfactants. In some embodiments, the cationic surfactants comprise polyquaternium, such as polyquaternium-10, polyquaternium-7 and polyquaternium-73, Guar hydroxypropyltrimonium chloride, behenamidopropyl dimethylamine, polyquaternium-37, Behentrimonium chloride, Brassicamidopropyl Dimethylamine, Stearamidopropyl Dimethylamine, Polyquaternium-16, Disunfloweroylethyl Dimonium Chloride, Cetrimonium Chloride, Hydroxyethyl Cetearamidopropyldimonium Chloride, and Polyquaternium-11.

In some embodiments, a hair composition of the application further comprises one or more non-ionic surfactants. In some embodiments, the non-ionic surfactants comprise lauryl glucoside, C12-20 alkyl glucoside, Sunfloweroyl methylglucamide.

In some embodiments, a hair composition of the application further comprises one or more amphoteric surfactants. In some embodiments, the amphoteric surfactants comprise cocamidopropyl hydroxysultaine, lauryl betaine, or a combination thereof.

In some embodiments, a hair composition of the application further comprises one or more anionic surfactants. In some embodiments, the anionic surfactants comprise sodium lauryl methyl isethionate, sodium cocoyl glutamate, disodium cocoyl glutamate, sodium lauroyl lactylate, sodium methyl cocoyl taurate, sodium lauroyl sarcosinate, or a combination thereof.

In some embodiments, a hair composition of the application further comprises one or more emollients. In some embodiments, the emollients comprise Glycol Distearate, Jojoba Wax PEG-120 esters, Dimethicone, Ethylhexyl palmitate, Olea europaea (olive) fruit unsaponifiables, Caprylic/Capric Triglyceride (CCT), cetyl esters, Ethylhexyl Stearate, Castor oil propanediol esters, Crambe abyssinica seed oil phytosterol, Helianthus annuus (sunflower) seed oil, Sunflower Seed Oil Glycerides, Simmondsia chinensis (jojoba) butter, Astrocaryum murumuru Seed butter, Plukenetia volubilis seed oil, Salvia hispanica seed oil, Polycitronellol Acetate, Undecane, Tridecane, Simmondsia chinensis (Jojoba) seed oil, Crambe abyssinica (Abyssinian) Seed oil, Ricinus communis (castor) seed oil, Helianthus annuus (sunflower) seed oil unsaponifiables, Mangifera indica (mango) seed butter, Squalane, Astrocaryum vulgare Kernel oil, Dimethiconol, Argania spinosa kernel oil (Argan oil), Ethylhexyl Olivate, Glyceryl Ricinoleate, C₁₃₋₁₅ Alkane, Cocos nucifera (coconut) oil, Avocado oil, Amodimethicone, Gluconamido Amodimethicone, panthenol, propanediol, Copernicia cerifera (Carnauba) wax, or a combination thereof.

In some embodiments, a hair composition of the application further comprises one or more lubricants. In some embodiments, the lubricants comprise polycitronellol acetate, and cetyl esters.

In some embodiments, a hair composition of the application further comprises one or more thickening agents. In some embodiments, the thickening agents comprise acrylates copolymer, acrylamidopropyl trimonium chloride/acrylamide copolymer, Polyquaternium-73, or a combination thereof.

In some embodiments, a hair composition of the application further comprises one or more chelating agents. In some embodiments, the chelating agents comprise EDTA.

In some embodiments, a hair composition of the application further comprises one or more moisturizers. In some embodiments, the moisturizers comprise glycerin. In some embodiments, the moisturizers comprise Cocos nucifera (Coconut) Oil. In some embodiments, the moisturizers comprise Jojoba Oil.

In some embodiments, a hair composition of the application further comprises one or more exfoliators. In some embodiments, the exfoliators comprise Lactobacillus/Pumpkin Fruit Ferment Filtrate.

In some embodiments, a hair composition of the application further comprises one or more acids. In some embodiments, the acids comprise citric acid, tartaric acid, caprylic acid, itaconic acid, or a combination thereof.

In some embodiments, a hair composition of the application further comprises water.

In some embodiments, a hair composition of the application has a PH of 4-6, such as a pH of 4, 4.5, 5, 5.5 or 6.

In some embodiments, a hair composition of the application further comprises betaine.

In some embodiments, a hair composition of the application further comprises one or more extracts. In some embodiments, the extracts comprise Raphanus sativus (Radish) seed extract, Rosmarinus officinales (rosemary) leaf extract, Tetrahexyldecyl Ascorbate, Caesalpina spinosa Fruit extract, Copernicia cerifera (Carnauba) Wax, or Helianthus annuus (sunflower) sprout extract.

In some embodiments, a hair composition of the application further comprises other cosmetically acceptable hair care ingredients selected from the group consisting of solubilizers, preservatives, surfactants, carriers, dyes, antioxidants, fillers, surfactants, UVA and/or UVB filters, fragrances, thickeners, humectants, whitening agents, antimycotic agents, antiparasitic agents, antimicrobial agents, external analgesics, sunscreens, photoprotectors, keratolytic agents, detergents, surfactants, moisturizers, nutrients, vitamins, energy enhancers, antiperspiration agents, astringents, deodorants, hair removers, firming agents, anti-callous agents, anti-static agents. In some embodiments, the cosmetically acceptable hair care ingredients are selected from the group consisting of emulsifier, preservative and solubilizer. Examples of cosmetically acceptable ingredients and/or excipients can be found in handbooks and pharmacopoeias for each country. Non-exhaustive examples of pharmacopeia are the American Pharmacopeia (“United States Pharmacopeia”), the European Pharmacopeia and the International Pharmacopeia.

In some embodiments, a hair composition of the application further comprises other hair care ingredients. The ingredients include, but are not limited to, a rheology modifier, a surfactant, an auxiliary fixative, a solvent, water, a conditioner, a propellant, a neutralizing agent, fragrance, a fragrance solubilizer, a thickener, preservative, an emulsifier, emollient, humectant, colorant, wax, fatty acid soap, suspending aids, vitamins, hair growth promoters, self-tanning agents, sunscreens, anti-dandruff agents, anti-inflammatory compounds, analgesics, antiperspirant agents, deodorant agents, hair fixatives, particulates, abrasives, moisturizers, antioxidants, keratolytic agents, anti-static agents, foam boosters, hydrotropes, solubilizing agents, chelating agents, antimicrobial agents, antifungal agents, pH adjusting agents, chelating agents, buffering agents, botanicals, oxidizing agents, reducing agents, hair bleaching agents, pigments, anticaries, anti-tartar agents, anti-plaque agents, hair and skin conditioning agents, skin lighteners, anti-aging compounds, anti-wrinkle compounds, hair coloring treatment agents, hair color protecting agents, hair styling products, anti-cellulite compounds, anti-acne compounds, and hair colorants.

In some embodiments, a hair composition of the application includes one or more hair conditioners which repair and improve the properties of hair, such as elasticity, resistance, texture (i.e., softness, smoothness, etc.), and appearance. In some embodiments, the hair composition comprises one or more hair conditioners selected from: panthenol, salicylic acid, sodium salicylate, carnosine, tetrahexyl decyl ascorbate, hyaluronic acid/sodium hyaluronate, hydrolyzed hyaluronic acid, sarcosine, PC A, arginine PC A, brassicyl valinate esylate, brassicyl isoleucinate esylate, brassica alcohol, glycolipids, glycosphingolipids, sphinganine, Rubus ideaus (raspberry) leaf extract, bis-ethyl(isostearylimidazoline) iso stearamide, Camellia sinensis leaf extract, Carthamus tinctorius (safflower) seed extract, Glycine soja (soybean) seed extract, Elaeis guineensis (palm) fruit extract, Oryza sativa (rice) extract, hydrolyzed Adansonia digitata seed extract, hydrolyzed corn starch, behenyl/stearyl aminopropanediol esters, C₁₀₋₄₀ isoalkylamidopropylethyldimonium ethosulfate, Linum usitatissimum (linseed) extract, Salvia hispanica seed extract, Astrocaryum mururmuru fruit extract, Moringa oleifera seed extract, cinnamidopropyltrimonium chloride, hydroxypropyl guar hydroxypropyltrimonium chloride, Trifolium pratense (clover) flower extract, Opuntia ficus-indica stem extract, Kappaphycus alvarezii extract, Laminaria saccharina extract, guar hydroxypropyltriammonium chloride, bis-diglyceryl polyacyladipate-2, hydroxypropylgluconamide, hydroxypropylammonium gluconate, biosaccharide gum-4, Prunus amygdalus dulcic (sweet almond) seed extract, soybean glycerides, Butyrospermum parkii (shea) butter unsaponifiables, hydrolyzed chestnut extract, glyceryl rosinate, sodium methoxy peg-16 maleate/styrene sulfonate copolymer, panthenyl hydroxypropyl steardimonium chloride, and any combinations thereof.

In some embodiments, a hair composition of the application includes one or more conditioners which help retain moisture. In some embodiments, a hair composition of the application comprises one or more conditioners selected from: niacinamide, allantoin, maltooligosyl glucoside, hydrogenated starch hydrolysate, Opuntia ficus-indica stem extract, sodium hyaluronate crosspolymer, jojoba esters, jojoba wax PEG-120 esters, jojoba wax PEG-80 esters, hydrolyzed jojoba esters, oleic acid, linoleic acid, linolenic acid, lactobionic acid, ursolic acid/sodium ursolate, nordihydroguaiaretic acid, ceramides, fructooligosaccharides, galactoarabinan, schizophyllan, Anetholea anisate leaf extract, Tetraselmis suecica extract, saccharide isomerase, Alteromonas ferment extract, Pseudoalteromonas ferment extract, Corylus avellana leaf extract, Phyllanthus emblica fruit extract, Betula alba juice, Saccharum officinarum (sugarcane) extract, Rhodomyrtus tomentosa fruit extract, Hedychium coronarium root extract, Epilobium angustifolium flower/leaf/stem extract, Tremella fuciformis sporocarp extract, alphaglucan oligosaccharide, and any combinations thereof.

In some embodiments, the conditioning agents can be silicones, organic conditioning oils, natural and synthetic waxes, and cationic polymers.

In some embodiments, a hair composition of the application includes one or more cleansing agents that help to clean the hair but do not remove or replace any essential oils. In some embodiments, a hair composition of the application comprises one or more cleansing agents selected from: an anionic surfactant, an amphoteric surfactant, a non-ionic surfactant, a cationic surfactant, a biosurfactant, and any combinations thereof.

Surfactants, as cleaning agents, are also ingredients routinely included in hair products, providing foaming and detergent properties. Typically, dirt is excess of sebum, produced by scalp and dirt adsorbed to this sebum. Surfactants weaken the physicochemical adhesive force that binds lipid residues to the hair, transfer it into the aqueous rinse, and disperse them, avoiding redeposition on the hair fiber. Surfactants have, generally, a tail of fatty hydrocarbons and a polar head. In contact with water, they attain the structure formation of a micelle, with hydrophilic exterior and hydrophobic interior, where the residues are trapped and kept in a dispersed form within the aqueous rinse.

In some embodiments, a hair composition of the application includes one or more surfactant of an anionic surfactant, a cationic surfactant, an amphoteric and zwitterionic surfactant, a nonionic surfactant, a biosurfactant, or combinations thereof. The anionics are the primary cleansing agents, while the cationics, amphoterics and nonionics are additives that give certain product attributes.

In some embodiments, a hair composition of the application further comprises one or more anionic surfactants that include, but are not limited to, the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule. In some embodiments, the anionic surfactants include, but are not limited to, sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. In some embodiments, the anionic surfactants comprise sodium lauryl sulphate, triethanolamine monolauryl phosphate, sodium lauryl ether sulphate 1 EO, 2EO and 3EO, ammonium lauryl sulphate and ammonium lauryl ether sulphate 1EO, 2EO and 3EO. In some embodiments, the anionic surfactants comprise sodium methyl 2-sulfolaurate, sodium lauryl sulfoacetate, disodium 2-sulfolaurate, alkylbenzene sulfonates, ammonium lauryl sulfate, ammonium xylenesulfonate, sodium laureth sulfate, sodium lauryl sulfate, sodium lauryl sulfoacetate, sodium 2-sulfolaurate, sodium myreth sulfate, sodium xylenesulfonate, sodium C₁₄₋₁₆ olefin sulfonate, sodium C₁₄₋₁₆ alpha olefin sulfonate, disodium lauryl sulfosuccinate, sodium methyl cocoyl taurate, sodium cocoyl isethionate, sodium lauryl isethionate, sodium cocoyl sarcosinate, sodium lauroyl sarcosinate, sodium myristoyl sarcosinate, lauroyl sarcosine, cocoyl sarcosine, myristoyl sarcosine, oleoyl sarcosine, myristoyl/stearoyl sarcosine, sodium lauroyl glutamate, sodium cocoyl glutamate, disodium cocoyl glutamate, cocoyl glutamic acid, lauroyl glutamic acid, potassium cocoyl glutamate, potassium lauroyl glutamate, potassium myristoyl glutamate, sodium myristoyl glutamate, sodium stearoyl glutamate, disodium stearoyl glutamate, sodium palmoyl glutamate, sodium lauroyl lactylate, sodium astrocaryum murumuruate, sodium laurylglucosides hydroxypropyl sulfonate, disodium laurylglucosides hydroxypropyl citrate, sodium hydroxypropyl sulfonate laurylglucoside crosspolymer, dioctyl sodium sulfosuccinate, or any combinations thereof. In some embodiments, the anionic surfactants comprise one or more alkoxylated monoacids, alkyl sulfosuccinates, alkyl ether sulfosuccinates, or mixtures thereof. In some embodiments, the anionic surfactants are chosen from ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, salts thereof, or mixtures thereof. In some embodiments, the anionic surfactants comprise one or more acyl isethionate surfactants, selected from sodium cocoyl isethionate, sodium cocoyl methyl isethionate, sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, a blend of stearic acid and sodium cocoyl isethionate, ammonium cocoyl isethionate, ammonium cocoyl methyl isethionate, or mixtures thereof. In some embodiments, the hair compositions comprise sodium cocoyl isethionate.

In some embodiments, a hair composition of the application further comprises one or more cationic surfactants, i.e., surfactants with a positive charge on their hydrophilic end. The cationic surfactants useful for the application include, but are not limited to, ethyl lauroyl arginate HCl, polyquaternium-10, polyquaternium-7, polyquatemium-11, polyquaternium-16, polyquatemium-57, quaternium-15, quatemium-18, quaternium-22, quaternium-87, guar hydroxypropyltrimonium chloride, polyquaternium-73, Guar hydroxypropyltrimonium chloride, behenamidopropyl dimethylamine, polyquaternium-37, behentrimonium chloride, brassicamidopropyl dimethylamine, stearamidopropyl dimethylamine, disunfloweroylethyl dimonium chloride, cetrimonium chloride, hydroxyethyl cetearamidopropyldimonium chloride, stearimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide, and combinations thereof.

In some embodiments, a hair composition of the application further comprises one or more amphoteric and/or zwitterionic surfactants, wherein the amphoteric and/or zwitterionic surfactants are a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The amphoteric and/or zwitterionic surfactants can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof. In some embodiments, the amphoteric and/or zwitterionic surfactants include, but are not limited to, alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms, lauryl amine oxide, cocodimethyl sulphopropyl betaine and preferably lauryl betaine, cocamidopropyl betaine, or sodium cocamphopropionate. In some embodiments, the amphoteric surfactants are selected from: cocamindopropyl hydroxysultaine, cocamidopropyl betaine, coco betaine, betaine, cocoamphoacetate, cocoamphodipropionate, disodium cocoamphodiacetate, disodium cocoamphodipropionate, lauryl betaine, lauramidopropyl betaine, lauramidopropyl hydroxysultaine, lauryl hydroxysultaine, sodium dilauramidoglutamide lysine, sodium cocoamphopropionate, lauroamphoacetate, and any combinations thereof.

In some embodiments, a hair composition of the application further comprises one or more nonionic surfactants. In some embodiment, the nonionic surfactants comprise condensation products of aliphatic (C₈-C₁₈) primary or secondary linear or branched chain alcohols or phenols with alkylene oxides. In some embodiments, the alkylene oxides comprise ethylene oxides. In some embodiments, the ethylene oxides have 6 to 30 ethylene oxide groups. In some embodiments, the nonionic surfactants comprise mono- or di-alkyl alkanolamides. In another embodiments, the nonionic surfactants include coco mono- or di-ethanolamide and coco mono-isopropanolamide.

In some embodiments, the nonionic surfactants comprise the alkyl polyglycosides (APGs). In some embodiments, the APGs comprise an alkyl group connected (optionally via a bridging group) to a block of one or more glycosyl groups. In some embodiments, APGs are defined by the following formula: RO-(G)ₙ, wherein R is a branched or straight chain alkyl group, which can be saturated or unsaturated, and G is a saccharide group. In some embodiments, R represents a mean alkyl chain length of from C₅ to C₂₀. In some embodiments, R represents a mean alkyl chain length of from C₈ to C₁₂. In some embodiments, R represents a mean alkyl chain length of from C₉ to C₁₀. In some embodiments, R represents a mean alkyl chain length of C₉. In some embodiments, R represents a mean alkyl chain length of from C₁₀. In some embodiments, G is selected from C₅ or C₆ monosaccharide residues. In some embodiments, G is a glucoside. In some embodiments, G is selected from the glucose, xylose, lactose, fructose, mannose and derivatives thereof. In some embodiments, G is glucose. In some embodiments, G is xylose. In some embodiments, G is lactose. In some embodiments, G is fructose. In some embodiments, G is mannose. In some embodiments, the degree of polymerization, n, may have a value of from 1 to about 10 or more. In some embodiments, n is from about 1.1 to about 2. In some embodiments, n is from about 1.1 to about 1.3. In some embodiments, n is from about 1.3 to about 1.5. In some embodiments, n is from about 1.5 to about 2.0. In some embodiments, n is about 1.3. In some embodiments, n is about 1.4. In some embodiments, n is about 1.5.

In some embodiments, the nonionic surfactant comprises alkyl polyglycosides, which are commercially available, and include those materials identified as: Oramix™ NS10 (available from Seppic); Plantaren™ 1200 and Plantaren™ 2000 (available from Henkel).

In certain embodiments, the nonionic surfactant can be chosen from alkyl and polyalkyl esters of polyethylene oxide, alkyl and polyalkyl ethers of polyethylene oxide, optionally polyoxyethylenated alkyl and polyalkyl esters of sorbitan, optionally polyoxyethylenated alkyl and polyalkyl ethers of sorbitan, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof. Exemplary and nonlimiting alkyl and polyalkyl esters of polyethylene oxide include those containing at least one C₈-C₃₀ alkyl radical, with a number of ethylene oxide (EO) units ranging from 2 to 200. Mention may be made, for example, of PEG-20 stearate, PEG-40 stearate, PEG-100 stearate, PEG-20 laurate, PEG-8 laurate, PEG-40 laurate, PEG-55 propylene glycol oleate, PEG-150 distearate, PEG-7 cocoate, PEG-9 cococate, PEG-8 oleate, PEG-10 oleate, and PEG-40 hydrogenated castor oil. Exemplary and nonlimiting alkyl and polyalkyl ethers of polyethylene oxide) include those containing at least one C8-C30 alkyl radical, with a number of ethylene oxide (EO) units ranging from 3 to 200. Mention may be made, for example, of laureth-3, laureth-4, laureth-5, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C₁₂₋₁₅ pareth-3, C₁₂₋₁₃ pareth-4, C₁₂₋₁₃ pareth-23, trideceth-3, trideceth-4, trideceth-5, trideceth-6, trideceth-7, Trideceth-8, trideceth-10, and mixtures thereof. Exemplary and nonlimiting polyoxyethylenated alkyl and polyalkyl esters of sorbitan include those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

In some embodiments, the non-ionic surfactant is selected from: decyl glucoside, lauryl glucoside, coco-glucoside, cocoyl methyl glucamide, capryloyl/caproyl methyl glucamide, lauroyl/myristoyl methyl glucamide, cocamidopropylamine oxide, decylamine oxide, and combinations thereof. In some embodiments, the non-ionic surfactant is a solubilizer, emulsifier, stabilizer, emollient, thickener, and any combinations thereof.

In some embodiments, the non-ionic surfactant comprises lauryl lactyl lactate, Trideceth-12, Trideceth-7, Trideceth-8, and Crambe abyssinica seed oil phytosterol esters.

In some embodiments, a hair composition of the application further comprises one or more cleansing agents, such as a biosurfactant selected from: Sapindus mukorossi (soapnut) extract, saponin, lipopeptide, potassium cocoyl hydrolyzed oat protein, starmella bombicola/Brassica oil ferment, glycoside, glycolipid, rhamnolipid, phospholipid, and any combinations thereof.

In some embodiments, a hair composition of the application includes co-surfactants. In some embodiments, those co-surfactants help impart aesthetic, physical or cleansing properties to the composition.

In some embodiments, a hair composition of the application includes cosmetically or dermatologically acceptable excipients. The cosmetically or dermatologically acceptable excipients includes, but are not limited to, preservatives, antioxidants, thickeners, chelating agents, co-solvents, sunscreen agents, vitamins, dyes, hair coloring agents, proteins, amino acids, organic polymers, natural extracts such as plant extracts, humectants, fragrances, perfumes, oils, emollients, lubricants, butters, penetrants, thickeners, viscosity modifiers, polymers, resins, hair fixatives, silicones, film formers, surfactants, detergents, emulsifiers, opacifying agents, volatiles, propellants, liquid vehicles, carriers, salts, pH adjusting agents (buffers), neutralizing agents, buffers, hair conditioning agents, anti-static agents, anti-frizz agents, anti-dandruff agents, absorbents, and any combinations thereof. In some embodiments, additives are selected from the group consisting of preservatives, antimicrobial agents, emulsifiers, solvent and wetting agents.

A hair composition of the application can optionally further comprise at least one thickening agent (also referred to as rheology or viscosity modifying agents). As non-limiting examples, polysaccharide-based thickeners, which are polymers having monosaccharides or disaccharides as base units, may be chosen. The polysaccharide thickeners which can be used in the compositions according to the application include, by way of example only, gums, celluloses, starches, or mixtures thereof. In some embodiments, a hair composition of the application includes a rheology modifier or the rheology modifying polymer, such as a polymer selected from the group consisting of carboxymethyl cellulose, hydroxyethyl cellulose, hydroxypropyl cellulose, hydroxypropylmethyl cellulose, hydroxypropyl guar, hydroxymethyl hydroxyethyl cellulose, and combinations thereof.

In some embodiments, the one or more cosmetically or dermatologically acceptable excipients useful for a hair composition of the application comprise one or more thickeners, such as one or more selected from: lauryl lactate, hydroxypropyl starch phosphate, acrylates copolymer, ammonium acryloyldimethyltaurate, sodium acryloyldimethyltaurate/VP copolymer, sodium acrylate/sodium acryloyldimethyl taurate copolymer, behenyl alcohol, stearic acid, sodium polyacrylate, polyacrylate crosspolymer-11, carbomer, acrylates/C10-30 alkyl acrylate crosspolymer, acrylates copolymer, acrylamidopropyl trimonium chloride/acrylamide copolymer, Polyquaternium-73, hydroxyethyl acrylate/sodiumacryloyldimethyltaurate copolymer, lithium magnesium sodium silicate, sodium magnesium fluorosilicate, glycol distearate, hydrogenated castor oil, hydrogenated castor oil hydroxystearate, hydrogenated castor oil isostearate, hydrogenated castor oil stearate, hydrogenated castor PEG-8 esters, Copernicia cerifera (Carnauba) wax, and any combinations thereof. Non-limiting examples of gums include acacia, agar, algin, alginic acid, ammonium alginate, amylopectin, calcium alginate, calcium carrageenan, cetyl hydroxyethylcellulose, hydropropylcelluloses, cetyl alcohol, cetearyl alcohol, carnitine, carrageenan, dextrin, gelatin, gellan gum, guar gum, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosacharide gum. Modified gums or derivatives of gums may also be used, such as, for example, deacylated gellan gum, welan gum, or hydroxypropylated guar gum, such as Jaguar HP 105 sold by Rhodia. Non-limiting examples of celluloses include hydroxyalkylcelluloses, such as hydroxyethylcellulose, hydroxypropylmethylcellulose, cetyl hydroxyethylcellulose, or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C₈-C₂₂ alkyl groups, such as the product NATROSOL™ Plus Grade 330 CS (C₁₆ alkyls), sold by Aquaion, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose. Non-limiting examples of starches include modified starches, starch-based polymers, methylhydroxypropyl starch, potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In some embodiments, the one or more cosmetically or dermatologically acceptable excipients comprise one or more emulsifiers, such as that selected from: caprylic/capric/diglyceryl succinate, C14-16 glycol palmitate, C18-20 glycol isostearate, cocamidopropyl lauryl ether, dipentaerythrityl hydroxystearate, dipentaerythrityl hydroxyisostearate, dipentaerythrityl hexacaprate/caprylate, palm kernel glycerides, lecithin, hydrogenated lecithin, phosphatidylcholine, C₁₂₋₁₆ alcohols, palmitic acid, glyceryl stearate, cetyl alcohol, stearyl alcohol, behenyl alcohol, palmitic acid, stearic acid, glyceryl behenate, glyceryl stearate, glyceryl stearate citrate, glyceryl oleate citrate, sorbitan laurate, lauryl laurate, sorbitan olivate, cetearyl olivate, sodium stearoyl lactylate, sodium stearoyl glutamate, sodium cocoyl glutamate, caprylyl/capryl glucoside, polyhydroxy stearic acid, phenethyl alcohol, sodium surfactin, polyglycerin-3, polyglyceryl-3 distearate, polyglyceryl-3 diisostearate, polyglyceryl-3 cocoate, polyglyceryl-3 dicitrate/stearate, polyglyceryl-3 beeswax, polyglyceryl-3 polyricinoleate, polyglyceryl-4 oleate, polyglyceryl-4 caprate, polylgylceryl-4 laurate, polyglyceryl-6 caprylate, polyglyceryl-6 stearate, polyglyceryl-6 distearate, polyglyceryl-6 behenate, polyglyceryl-6 polyhydroxystearate, polyglyceryl-6 oleate, polyglyceryl-6 ricinoleate, polyglyceryl-6 polyricinoleate, polyglyceryl-10 laurate, candelilla/jojoba/rice bran polyglyceryl-3 esters, stearoyl glutamic acid, Polysorbate-20, polyglyceryl-10 laurate, polyglyceryl-6 caprylate, polyglyceryl-3 cocoate, polyglyceryl-4 caprate, polyglyceryl-6 ricinoleate, polyglyceryl-6 caprylate, sodium surfactin, C14-22 alcohols, stearyl alcohol, palmitic acid, stearic acid, Crambe abyssinica seed oil phytosterol esters, Trideceth-12, and any combinations thereof.

In some embodiments, a hair composition of the application can optionally further comprise one or more chelating agents, which can improve stabilization of surfactant formulation foam during the user experience and can even remove some metals from the hair itself, which can lead to softer-feeling hair without the use of conditioning agents. To avoid lack of use and ensuing decrease in applied peptide benefits, the clarifying formula must avoid the consumer-associated perception of unpleasant “stripping” this can be achieved by the combination of peptides and salicylic acid in formulation to both minimize swelling of the cuticle that could lead to more prominent scales, tangling, breakage, etc. and prevent protein loss during the more intensely clarifying shampoo process. In some embodiments, the one or more cosmetically or dermatologically acceptable excipients comprise one or more chelating agents selected from, but not limited to: phytic acid, sodium phytate, caprylhydroxamic acid, trisodium dicarboxymethyl alaninate, gluconic acid, sodium gluconate, pentasodium pentetate, sodium carboxymethyl inulin, tetrasodium glutamate diacetate, trisodium ethylenediamine succinate, sodium thiosulfate, diiospropyl oxalate, disodium EDTA, disodium EDTA-copper, HEDTA, oxalic acid, potassium citrate, sodium citrate, dodium oxalate, TEA-EDTA, tetrasodium EDTA, trisodium EDTA, trisodium HEDTA, and any combinations thereof.

In some embodiments, the one or more cosmetically or dermatologically acceptable excipients useful for a hair composition of the application comprise one or more preservatives, such as those selected from: benzyl alcohol, benzoic acid, butyl paraben, diazolidinyl urea, DMDM hydantoin, ethyl paraben, imidazolidinyl urea, iodopropynyl butylcarbamate, isobutyl paraben, methyl paraben, methylchloroisothiazolinone, methylisothiazolinone, phenoxyethanol, propyl paraben, sodium benzoate, potassium sorbate, ethylhexylglycerin, gluconolactone, xylityl sesquicaprylate, caprylic acid, itaconic acid, ethyl lauroyl arginate, and any combinations thereof.

In some embodiments, the one or more cosmetically or dermatologically acceptable excipients useful for a hair composition of the application comprise one or more anti-static agents. In some embodiments, the anti-static agent is a cationic surfactant selected from: quaternary ammonium compounds, behentrimonium chloride, behentrimonium methosulfate, cetrimonium chloride, cetrimonium methosulfate, cetyltrimethyl ammonium chloride (CTAC), benzalkonium chloride, benzethonium chloride, cinnamidopropyltrimonium chloride, cocotrimonium chloride, dicetyldimonium chloride, dicocodimonium chloride, laurtrimonium chloride, stearamidopropyl dimethylamine, steartrimonium chloride, stearalkonium chloride, tricetyldimonium chloride, PCA ethyl cocoyl arginate, C₁₀₋₄₀ isoalkylamidopropylethyldimonium ethosulfate, dipalmitoylethyl hydroxyethylmonium methosulfate, shea butteramidopropyltrimonium chloride, acrylamidopropyl trimonium chloride/acrylamide copolymer, and any combinations thereof.

In some embodiments, the one or more cosmetically or dermatologically acceptable excipients useful for a hair composition of the application comprise one or more naturally derived polymers selected from: xanthan gum, pullulan, algin, tetragonoloba (guar) gum, sclerotium gum, acacia Senegal gum, cellulose, cellulose gum, carboxymethylcellulose, carbomer, hydroxypropyl starch phosphate, guar hydroxypropyltrimonium chloride, carboxymethyl hydroxyethyl cellulose, carboxymethyl hydroxypropyl guar, cellulose, ethyl cellulose, hydroxybutyl methylcellulose, hydroxyethylcellulose, hydroxymethylcellulose, lauryl polyglucose, and any combinations thereof.

In some embodiments, the one or more cosmetically or dermatologically acceptable excipients useful for a hair composition of the application comprise one or more amino acids such as L-cysteine. In some embodiments, the one or more amino acids are selected from arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. In some embodiments, the amino acid is arginine.

In some embodiments, a hair composition of the application can optionally further comprise one or more humectants, such as those selected from: 1,2,6 hexanetriol, dipropylene glycol, glycerin, butylene glycol, caprylyl glycol, pentylene glycol, isopentyldiol, hexylene glycol, hydroxyacetophenone, panthenol, phytantriol, propylene glycol, propanediol, sodium PCA, sorbitol, triethylene glycol, polyglyceryl sorbitol, glucose, fructose, mannitol, xylitol, inositol, trehalose, polydextrose, potassium PCA, hydrogenated honey, hyaluronic acid, inositol, erythritol, capryl glycol, acetamidoethoxyethanol, and any combinations thereof.

In some embodiments, a hair composition of the application further comprises one or more humectants. In some embodiments, the humectant comprises Glycol Distearate, Jojoba Wax PEG-120 esters, Dimethicone, Ethylhexyl palmitate, Olea europaea (olive) fruit unsaponifiables, Caprylic/Capric Triglyceride (CCT), Ethylbexyl Stearate, Castor oil propanediol esters, Crambe abyssinica seed oil phytosterol, Sunflower Seed Oil Glycerides, Simiondsia chinensis (jojoba) butter, Astrocaryum murunmuru Seed butter, Plukenetia volubilis seed oil, Salvia hispanica seed oil, Polycitronellol Acetate, Undecane, Tridecane, Simmondsia chinensis (Jojoba) seed oil, Crambe abyssinica (Abyssinian) Seed oil, Ricinus communis (castor) seed oil, Helianthus annuus (sunflower) seed oil, Helianthus annuus (sunflower) seed oil unsaponifiables, Mangifera indica (mango) seed butter, Squalane, Astrocaryum vulgare Kernel oil, Dimethiconol, Argania spinosa kernel oil (Argan oil), Ethylhexyl Olivate, Glyceryl Ricinoleate, C₁₃₋₁₅ Alkane, Cocos nucifera (coconut) oil, Avocado oil, Amodimethicone, Gluconamido Amodimethicone, Copernicia cerifera (Carnauba) wax, or a combination thereof.

In some embodiments, a hair composition of the application can optionally further comprise one or more cationic polymers, i.e., polymers bearing a positive charge or incorporating cationic entities in their structure. Non-limiting examples cationic polymers useful for a hair composition of the application include cationic cellulose derivatives (e.g., polyquaternium-10), quaternized hydroxyethyl cellulose, cationic starch derivatives, cationic guar gum derivatives, cationic proteins and cationic protein hydrolysates, quaternary diammonium polymers, copolymers of acrylamide and dimethyldiallyammonium chloride, polyquaterniums (e.g., polyquaternium-10), polyquaternium-7, polyquatemium-11, polyquatemium-57, quaternium-15, quatemium-18, quaternium-22, quaternium-87, guar hydroxypropyltrimonium chloride, cationic guar gum derivative, amidoamine, monoalkyl quaternary amine, dialkyl quaternary amine, polyquaternium compound, or salts thereof, oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

In some embodiments, the cationic polymers useful for a hair composition of the application comprise one or more including, but not limited to, polyquaterium-10, cetrimonium chloride, behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, Quaterium-8, or mixtures thereof.

In some embodiments, the cationic polymers useful for a hair composition of the application comprise one or more selected from hydroxypropyl guar hydroxypropyltrimonium chloride, guar hydroxypropyltrimonium chloride, polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, or polyquaternium-94, or mixtures thereof.

In some embodiments, a hair composition of the application can optionally further comprise one or more nonionic polymers, i.e., polymers lacking a net electrical charge. Non-limiting examples of nonionic polymers useful for a hair composition of the application include polysaccharides, polysaccharide derivatives (e.g., guar gum, guar derivatives, cellulose gum, cellulose derivatives, starch, starch derivatives), homopolymers and copolymers of ethylene oxide having a molar mass equal to or greater than 10,000 g/mol, polyvinyl alcohols, homopolymers and copolymers of vinylpyrrolidone, homopolymers and copolymers of vinylcaprolactam, homopolymers and copolymers of polyvinyl methyl ether, and mixtures thereof. Many nonionic polymers have thickening properties and are sometimes referred to as “nonionic thickening polymers.” Nonionic thickening polymers can be useful in compositions of the instant disclosure, for example, when a certain degree of thickening is desired.

In some embodiments, a hair composition of the application can optionally further comprise one or more glucamide(s). The glucamide useful for a hair composition of the application can be an acyl glucamides. For example, the glucamide(s) can be selected from acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide. The glucamide may be a C₁₀-C₁₈ N-alkyl (C₁-C₆) polyhydroxy fatty acid amides, such as the C₁₂-C₁₈ N-methyl glucamides, as described for example in WO 9206154 and U.S. Pat. No. 5,194,639, which are incorporated herein in their entireties for all purposes, and the N-alkoxy polyhydroxy fatty acid amides, such as C₁₀-C₁₈ N-(3-methoxypropyl) glucamide.

In some embodiments, a hair composition of the application can optionally further comprise one or more film formers, such as acrylamidopropyl trimonium chloride/acrylamide copolymer.

In some embodiments, a hair composition of the application can optionally further comprise one or more silicones, such as that selected from: amodimethicone, dimethicone cyclomethicone, cyclopentasiloxane, dimethicone, dimethicone copolyol, dimethiconol, hydrolyzed wheat protein hydroxypropyl polysiloxane, trimethylsilylamodimethicone, and any combinations thereof. In certain embodiments, a hair composition of the application is silicone-free, i.e., does not contain any silicone.

In some embodiments, a hair composition of the application can optionally further comprise one or more proteins or peptides, such as those selected from: cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, cocoyl hydrolyzed collagen, cocoyl hydrolyzed keratin, hydrolyzed keratin, hydrolyzed oat flour, hydrolyzed silk, hydrolyzed silk protein, hydrolyzed soy protein, hydrolyzed wheat protein, hydrolyzed quinoa, hydrolyzed elastin, hydrolyzed collagen, keratin, potassium cocoyl hydrolyzed collagen, and any combinations thereof.

In some embodiments, a hair composition of the application can optionally further comprise one or more vitamins, such as those selected from: retinol, retinyl palmitate, ascorbic acid, tocopherol, tocopherol acetate, and any combinations thereof. In some embodiments, a hair composition of the application can optionally further comprise a provitamin. In some embodiments, the provitamin is panthenol.

In some embodiments, a hair composition of the application can optionally further comprise one or more natural extracts, such as those selected from: Aloe barbadensis leaf extract, Yucca schidigera leaf extract, Yucca schidigera root extract, Camellia sinensis leaf extract, Equisetum arvense leaf extract, Hamamelis virginiana leaf extract, Macrocystis pyrifera kelp extract, Linum usitatissimum (Linseed) Seed Extract, Salvia hispanica (Chia) Seed Extract, Aphanus sativus (Radish) seed extract, Raphanus sativus (Radish) seed extract, Rosmarinus officinales (rosemary) leaf extract, Rosmarinus officinalis (Rosemary) Leaf Extract, Tetrahexyldecyl Ascorbate, Caesalpina spinosa Fruit extract, Helianthus annuus (sunflower) sprout extract, Plukenetia volubilis seed oil, Salvia hispanica seed oil, Astrocaryum vulgare kernel oil, and combinations thereof. In some embodiments, the natural extract is Linum usitatissimum (linseed) seed extract. In some embodiments, the natural extract is Salvia hispanica (chia) seed extract. In some embodiments, the natural extract is a combination of Linum usitatissimum (Linseed) seed extract and Salvia hispanica (Chia) seed extract.

In some embodiments, a hair composition of the application can optionally comprise one or more emollients, such as that selected from: isopropyl lauroyl sarcosinate, phenethyl benzoate, C₁₂₋₁₅ alkyl benzoate, caprylic/capric triglyceride (CCT), cetyl octanoate, octyldodecanol, dilinoleic acid/propanediol copolymer, isoamyl laurate, oleic acid, isohexadecane, squalane, isopropyl palmitate, polycitronellol, diheptyl succinate, capryloyl glycerin/sebacic acid copolymer, PPG-3 benzyl ether ethylhexanoate, hydrogenated vegetable oil, triheptanoin, diisopropyl sebacate, diisopropyl adipate, propylene glycol dibenzoate, propoxytetramethyl piperidinyl dimethicone, castor oil/IPDI copolymer, butylene glycol cocoate, butylene glycol dicaprylate/dicaprate (dispersant), propylene glycol dicaprylate/dicaprate, triolein (algae oil), propylene glycol dicaprylate/dicaprate, phytosteryl/octyldodecyl lauroyl glutamate, C₁₀₋₁₈ triglycerides, isostearyl alcohol, diisostearyl malate, isoamyl laurate, isoamyl cocoate, hemisqualane, polycitronellol acetate, ethyl macadamiate, coco-caprylate/caprate, coconut alkanes, C₉₋₁₂ alkane, C₁₃₋₁₅ alkane, C₁₃₋₁₆ isoalkane, neopentyl glycol diheptanoate, hydrogenated ethylhexyl olivate, hydrogenated olive oil unsaponifiables, undecane, tridecane, isododecane, caprylyl caprylate/caprate, cetyl stearate, cetearyl stearate, decyl oleate, glyceryl stearate, glyceryl adipate, glyceryl arachidate, glyceryl arachidonate, glyceryl behenate, glyceryl caprate, glyceryl caprylate, glyceryl caprylate/caprate, glyceryl citrate/lactate/linoleate/oleate, glyceryl cocoate, glyceryl diarachidate, glyceryl dibehenate, glyceryl dierucate, glyceryl dihydroxystearate, glyceryl diisopalmitate, glyceryl diisostearate, glyceryl dilaurate, glyceryl dilinoleate, glyceryl dimyristate, glyceryl dioleate, glyceryl dipalmitate, glyceryl dipalmitoleate, glyceryl diricinoleate, glyceryl distearate, glyceryl erucate, glycol stearate, glyceryl stearate, isocetyl stearate, isopropyl myristate, isopropyl palmitate, isopropyl stearate, isostearyl stearate, octyl palmitate, octyl stearate, propylene glycol dicaprylate/dicaprate, sorbitan benzoate, sorbitan caprylate, sorbitan isostearate, sorbitan laurate, sorbitan tristearate, stearyl stearate, tocopheryl linoleate, olive oil, avocado oil, coconut oil, sunflower oil, jojoba oil, argan oil, castor oil, almond oil, Sesamum indicum (sesame) seed oil, Prunus areniaca (apricot) kernel oil, Crambe abyssinica seed oil, Oryza sativa (rice) bran oil, Vitis vinifera (grape) seed oil, Passiflora edulis seed oil, meadowfoam seed oil, Butyrospermum parkii (shea) butter, Shorea robusta seed butter, murumuru butter, glycol distearate, Jojoba Wax PEG-120 Esters, lauryl lactyl lactate, and any combinations thereof.

In some embodiments, a hair composition of the application can optionally further comprise one or more antioxidants, such as those selected from: carnosine, tetrahexyl decyl ascorbate, bioflavonoids, lycopene, Daucus carota sativa root cell culture lysate, monoammonium glycyrrhizinate, alpha lipoic acid (thioctic acid), Leontopodium alpinum (edelweiss) extract, Rosmarinus officinalis (rosemary) leaf extract (WS), Vitis vinifera (grape) seed extract, Camellia sinensis leaf extract, Quercus robur wood extract, hydrolyzed olive fruit, Olea europea (olive) leaf extract, oleuropin, hydroxyacetophenone, Punica granatum bark/fruit extract, punicalagin, ellagic acid, polyphenols, epigallocatechin (EGCG), tannins, sulforaphane, resveratrol, nordihydroguaiaretic acid, and any combinations thereof.

In some embodiments, a hair composition of the application can optionally further comprise one or more buffers, such as those selected from: ascorbic acid, citric acid, lactic acid, glycolic acid, tartaric acid, malic acid, L-arginine, potassium hydroxide, sodium hydroxide, triethanolamine, sodium bicarbonate, and any combinations thereof.

In some embodiments, a hair composition of the application further comprises a buffer system to adjust the pH of the composition to about 2 to about 6. The buffer system can be any combination of one or more acid(s) and one or more base(s). Typically, the buffer system comprises one or more inorganic and/or an organic acid(s) and/or salt(s) thereof to provide the hair care composition with a pH value from about 2 to about 6 at 25° C. In some embodiments, the pH value can range from about 2 to about 5. In some embodiments, the pH value can range from about 2 to about 4. In some embodiments, the pH value can range from about 2 to about 3. In some embodiments, the pH value can range from about 3 to about 6. In some embodiments, the pH value can range from about 3 to about 5. In some embodiments, the pH value can range from about 3 to about 4. In some embodiments, the pH value can range from about 4 to about 6. In some embodiments, the pH value can range from about 4 to about 5. In some embodiments, the pH value of the hair composition can range from about 5 to about 6. In some embodiments, the buffering system comprises an inorganic acid selected from the group consisting of hydrogen chloride (HCl), sulfuric acid (H₂SO₄), nitric acid (HNO₃), phosphoric acid (H₃PO₄), and combinations thereof. In some embodiments, the buffering system comprises an organic acid selected from the group consisting of an alpha-hydroxy acid, a polycarboxylic acid, and combinations thereof. In some embodiments, the organic acid has an acidic functional group having a pKa of about 4.5 or less. In some embodiments, the organic has a second acidic functional group having a pKa of about 6 or less. The organic acid can have a molecular weight less than about 500 grams per mole (g/mol). For example, but not by way of limitation, the molecular weight of the organic acid can be from about 90 g/mol to about 400 g/mol, or from about 100 g/mol to about 300 g/mol, or from about 130 g/mol to about 250 g/mol, or from about 150 g/mol to about 200, or about 190 g/mol. The organic acid can be soluble in water in an amount greater than about 0.2 moles per liter at 25° C. For example, but not by way of limitation, the water solubility of the organic acid may be about 0.3 mol/L or more, or about 0.4 mol/L or more, or about 0.5 mol/L or more. In some embodiments, the organic acids can include, but are not limited to, lactic acid, citric acid, caprylic acid, tartaric acid, gluconolactive acid, pimelic acid, glyoxylic acid, aconitic acid, ethylenediaminetetraacetic acid, L-glutamic acid, malic acid, malonic acid, itaconic acid, and combinations thereof. In some embodiments, a salt of such an inorganic acid and an organic acid can include its alkali metal salts such as the sodium salt and the potassium salt; its ammonium salt; and alkanolamine salts such as the triethanolamine salt.

In some embodiments, the one or more cosmetically or dermatologically acceptable excipients useful for a hair composition of the application comprise one or more salts selected from: calcium chloride, magnesium chloride, magnesium sulfate, potassium chloride, potassium glycol sulfate, sodium chloride, and any combinations thereof.

In some embodiments, a hair composition of the application can optionally further comprise one or more aliphatic alcohols, such as those selected from: propylene glycol, lauric acid, palmitic acid, stearic acid, arachidic alcohol, behenyl alcohol, cetearyl alcohol, cetyl alcohol, isocetyl alcohol, isostearyl alcohol, isopropanol, lauryl alcohol, myristyl alcohol, stearyl alcohol, C30-50 alcohols, lanolin alcohol, panthenol, propanediol, and any combinations thereof.

In some embodiments, a hair composition of the application can optionally further comprise one or more perfuming ingredients. In some embodiments, the perfuming ingredient includes a fragrance. In some embodiments, the fragrance includes limonene.

In some embodiments, a hair composition of the application can optionally further comprise one or more exfoliators, such as those selected from alpha-hydroxy acids (glycolic acid, lactic acid, mandelic acid, malic acid, tartaric acid, and citric acid), beta hydroxy acids (salicylic acid), polyhydroxy acids (lactobionic acid, gluconolactone, and galactose) or enzymes (trypsin or collagenase). In some embodiments, the exfoliators useful for a hair composition of the application can be one or more of glycolic acid, lactic acid, mandelic acid, malic acid, tartaric acid, citric acid, salicylic acid, lactobionic acid, gluconolactone, galactose, trypsin, collagenase, or Lactobacillus/Pumpkin fruit ferment filtrate. In some embodiments, the exfoliator is Lactobacillus/Pumpkin fruit ferment filtrate.

In some embodiments, a hair composition of the application can optionally further comprise one or more antimicrobial agents, such as, but not limited to piroctone olamine, 3,4,4′-trichlorocarbanilide (trichlosan), triclocarban, caprylic acid, ethyl lauroyl arginate HCl, or xylityl sesquicaprylate.

In some embodiments, a hair composition of the application comprises one or more solvents. In some embodiments, the one or more solvents is selected from: water, dimethyl isosorbide, triethyl citrate, phenethyl benzoate, dicaprylyl maleate, dilinoleic acid/propanediol copolymer, lower alcohols containing from 1 to 6 carbon atoms, such as ethanol; polyols such as glycerine, glycols such as butylene glycol, isoprene glycol, propylene glycol, dipropylene glycol, propanediol, isopentyldiol, polyethylene glycols such as PEG-8, sorbitol, sugars such as glucose, fructose, maltose, lactose, sucrose; and any combinations thereof. In some embodiments, the solvent is water.

In some embodiments, a hair composition of the application can optionally further comprise one or more ingredients, such as those selected from adenosine, arbutin, niacinamide, retinol, vitamin C, vitamin C derivatives, vitamin A, vita E acetate, brown sea extract, antler extract, Ginseng extract, peony extract, angelica extract, bokryok extract, astragalus extract, licorice extract, ginger extract, jujube extract, cheongung extract, white extract, cinnamon, deer antler extract, green tea flakes, green tea extract, elm extract, angelica extract, carrot collagen, bamboo sap, Lactobacillus/pumpkin fermented extract, lemon extract, royal jelly extract, manroniae extract, guchi extract, mint extract, baobab extract, lettuce extract, lettuce extract, lettuce extract, mulberry extract, sake extract, cornus extract, lettuce extract, pomegranate extract, pomegranate extract extract, pine needle extract, swiss alpine herb extract, mugwort extract, sea silt extract, seaweed extract, Arnica montana extract, avocado oil, ivy extract, Aloe vera extract, aloe extract, fish extract, cucumber extract, cucumber herb extract, olive oil, olive extract, witch hazel extract, ginkgo leaf extract, impression extract, chamomile extract, caviar extract, coconut extract, coconut extract, chlorella extract, papaya extract, peppermint extract, grape seed oil, furellaria mira pica extract, fish collagen, deep sea water, herbal extract, red Ginseng extract, golden extract, rare extract, lavender blossom, lavender extract, sage extract, rosemary extract, yeast extract, pearl extract, acerola extract, tea tree extract, Ginseng extract, astragalus extract, yam root extract, upper extremity extract, jujube extract, or combination thereof.

In some embodiments, a hair composition of the application comprises 90% to 99.8%, by weight, of other ingredients, in addition to the 0.1%-5% (w/w) first component and 0.1%-5% (w/w) second component.

In some embodiments, a hair composition of the application comprises from 5% to 50%, by weight, of hair care conditioners. In some embodiments, the hair composition comprises from 10% to 30%, such as 10%, 15%, 20%, 25% or 30%, by weight, one or more conditioners.

In some embodiments, a hair composition of the application comprises 10% to 60%, by weight, of hair cleansing agents. In some embodiments, the hair composition comprises from 25% to 50%, such as 25%, 30%, 35%, 40%, 45% or 50%, by weight, of hair cleansing agents.

In some embodiments, the hair composition comprises from 0.01% to 10%, by weight, of one or more cationic surfactants. In some embodiments, the hair composition comprises from 0.01% to 2%, such as 0.01%, 0.05%, 0.1%, 0.2%, 0.3%, 0.4%, 0.5%, 0.6%, 0.7%, 0.8%, 0.9%, 1%, 1.2%, 1.4%, 1.6%, 1.8% or 2.0%, by weight, of cationic surfactants.

In some embodiments, the hair composition comprises from 0.1% to 30%, by weight, of one or more anionic surfactants. In some embodiments, the hair composition comprises from 1% to 15%, such as 1%, 2%, 3%, 4%, 5%, 6%, 7%, 8%, 9%, 10%, 11%, 12%, 13%, 14% or 15%, by weight, of one or more anionic surfactants.

In some embodiments, the hair composition comprises from 0.1% to 20%, by weight, of one or more non-ionic surfactants. In some embodiments, the hair composition comprises from 1% to 10%, by weight, such as 1%, 2%, 3%, 4%, 5%, 6%, 7%, 8%, 9% or 10%, of one or more non-ionic surfactants.

In some embodiments, the hair composition comprises from 0.1% to 30%, by weight, of one or more amphoteric surfactants. In some embodiments, the hair composition comprises from 5% to 15%, such as 5%, 6%, 7%, 8%, 9%, 10%, 11%, 12%, 13%, 14% or 15%, by weight, of one or more amphoteric surfactants.

In some embodiments, the hair composition comprises from 0.01% to 20%, by weight, of one or more thickening agents. In some embodiments, the hair composition comprises from 1% to 10%, such as 1%, 2%, 3%, 4%, 5%, 6%, 7%, 8%, 9% or 10%, by weight, of one or more thickening agents.

In some embodiments, the hair composition comprises from 0.01% to 2%, by weight, of one or more chelating agents. In some embodiments, the hair composition comprises from 0.01% to 1%, such as 0.01%, 0.05%, 0.1%, 0.2%, 0.3%, 0.4%, 0.5%, 0.6%, 0.7%, 0.8%, 0.9% or 1%, by weight, of one or more chelating agents.

In some embodiments, the hair composition comprises from 0.01% to 10%, by weight, of one or more preservatives. In some embodiments, the hair composition comprises from 0.1% to 5%, such as 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, 3%, 3.5%, 4%, 4.5% or 5%, by weight, of one or more preservatives.

In some embodiments, the hair composition comprises from 0.01% to 10%, by weight, of one or more amino acids. In some embodiments, the hair composition comprises from 0.01% to 2%, by weight, such as 0.01%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2%, of one or more amino acids.

In some embodiments, the hair composition comprises from 0.1% to 20%, by weight, of one or more humectants. In some embodiments, the hair composition comprises from 0.1% to 5%, by weight, such as 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4% or 5%, by weight, of one or more humectants.

In some embodiments, the hair composition comprises from 0.1% to 30%, by weight, of one or more cationic polymers. In some embodiments, the hair composition comprises from 0.1% to 5%, such as 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4% or 5%, by weight, of one or more cationic polymers.

In some embodiments, the hair composition comprises from 0.01% to 10%, by weight, of one or more natural extracts. In some embodiments, the hair composition comprises from 0.01% to 2%, such as 0.01%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2%, by weight, of one or more natural extracts.

In some embodiments, the hair composition comprises from 0.01% to 10%, by weight, of one or more emollients. In some embodiments, the hair composition comprises from 0.5% to 10%, such as 0.5%, 1%, 2%, 3%, 4%, 5%, 6%, 7%, 8%, 9% or 10%, by weight, of one or more emollients.

In some embodiments, the hair composition comprises from 0.01% to 10%, by weight, of one or more buffers/PH adjusters. In some embodiments, the hair composition comprises from 0.1% to 2%, such as 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2%, by weight, of one or more buffers/PH adjusters.

In some embodiments, the hair composition comprises from 0.01% to 10%, by weight, of one or more exfoliators. In some embodiments, the hair composition comprises 0.1% to 2%, such as 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2%, by weight, of one or more exfoliators.

In some embodiments, a hair composition of the application comprises from 0.001% to 3%, by weight, of one or more antimicrobial agents. In some embodiments, the hair composition comprises from 0.01% to 1%, such as 0.01%, 0.05%, 0.1%, 0.2%, 0.3%, 0.4%, 0.5%, 0.6%, 0.7%, 0.8%, 0.9%, or 1%, by weight, of one or more antimicrobial agents.

In some embodiments, a hair composition of the application comprises about 10% to 99%, by weight, of water, based on the total weight of the hair treatment composition. The hair treatment compositions can include about 10% to 90%, such as 10%, 20%, 30%, 40%, 45%, 50%, 55%, 60%, 65%, 70%, 75%, 80%, 85%, or 90%, by weight, of water, based on the total weight of the hair composition.

### Methods of Preparing

Another general aspect of the application relates to a method of preparing a hair composition of the application. The method comprises combining the components and/or ingredients of the composition to obtain the hair composition. In some embodiments, a method for producing a hair composition, comprising combining a first component described herein, a second component described herein, and one or more additional ingredients described herein to thereby obtaining a hair composition described herein.

A hair composition according to embodiments of the application can be used for any suitable applications. In some embodiments, a hair composition of the application is prepared as a shampoo, conditioner, mask, or leave-in hair treatment product.

### Methods of Use

Another general aspect of the application relates to a method of treating hair of a subject in need thereof, comprising applying to the hair a hair composition according to an embodiment of the application. In some embodiments, a method for strengthening hair comprises applying to the hair a hair composition according to an embodiment of the application. In some embodiments, a method for reducing and/or preventing hair breakage comprises applying to the hair a hair composition according to an embodiment of the application. The hair compositions can be applied to any suitable hair fibers, including but not limited to, virgin hair, hair treated with a chemical or mechanical hair treatment (e.g., bleached hairs, dyed hairs), and damaged or weakened hair fibers.

A hair composition of the application can be applied to the hair using known methods in view of the present disclosure. For example, 0.1 g to 30 g of the hair composition can be applied to 5.0 g of hair in the hair treatment method. The application of the hair composition to the hair can be carried out for 0.5 minute to 60 minutes. The hair composition can be rinsed off the hair immediately after the application or after a desirable delay period. The hair composition can also be left on the hair as a leave in or leave on product.

In some embodiments, a hair composition of the application is a shampoo for use to clean hair. In some embodiments, a hair composition of the application is a conditioner for use, e.g., to protect hair shafts from damage, to make hair shine, to soften hair, and/or to detangle hair and make it easier to manage. In some embodiments, a hair composition of the application is used to massage and detangle the hair. In some embodiments, the hair composition is used to massage and/or detangle the hair, e.g., during or after a hair treatment, such as hair bleaching. Locks of hair can be gently massaged with the hair composition for 1-40, 1-30 or 1-20 minutes. In some embodiments, locks of hair are gently massaged for 1-10 minutes, such as 1, 2, 3, 4, 5, 6, 7, 8, 9 or 10 minutes. In some embodiments, the wicks are rinsed for 1-40, 1-30, 1-20, 1-10 minutes, such as 1, 2, 3, 4, 5, 6, 7, 8, 9 or 10 minutes.

In some embodiments, a hair composition of the application is a leave-in product, such as a hair serum or hair styling product. In some embodiments, the composition is applied to dry locks of hair in an amount of 0.1 g-1.0 g of formulation, such as 0.1, 0.2, 0.3, 0.4, 0.5, 0.6, 0.7, 0.8, 0.9 or 1.0 g of the formulation for 1.0 g of hair along the entire length of the strands.

In some embodiments, the hair composition or formulation is applied before, during or after hair bleaching processes. In some embodiments, the hair composition or formulation is applied before hair bleaching processes. In some embodiments, the hair composition or formulation is applied during hair bleaching processes. In some embodiments, the hair composition or formulation is applied after hair bleaching processes. In some embodiments, the hair composition or formulation is applied before, during and after hair bleaching processes. In some embodiments, the hair composition or formulation is applied during and after hair bleaching processes. In some embodiments, the hair composition or formulation is applied during or after hair bleaching processes.

In some embodiments, the application is carried out during the discoloration process, the effect of the application being called a protective effect or after the hair discoloration process, this effect of the application being called the repairing effect. In some embodiments, the application occurs on hair that has not undergone hair bleaching processes. In some embodiments, the treatment method strengthens the hair. In some embodiments, the hair treatment method provides a protective effect on the cuticular surface. In some embodiments, the hair treatment method increases the shine of the hair.

In some embodiments, a treatment with a hair composition of the application promotes a protective effect, with at least 5%, 10%, 15%, 20%, 25%, 30%, 35%, 40%, 45%, 50% or more increase in the maximum breaking tension of bleached hair, as compared with the treatment with a control hair composition that does not contain the combination of the first component and second component used in the hair composition.

In some embodiments, a treatment with a hair composition of the application promotes a protective effect with at least 5%, 10%, 15%, 20%, 25%, 30%, 35%, 40%, 45%, 50% or more decrease in average combing strength, as compared with the treatment with a control hair composition that does not contain the combination of the first component and second component used in the hair composition.

In some embodiments, a treatment with a hair composition of the application promotes protective effect with at least 10%, 15%, 20%, 25%, 30%, 35%, 40%, 45%, 50%, 55%, 60%, 65%, 70% or more increase in gloss unit, as compared with the treatment with a control hair composition that does not contain the combination of the first component and second component used in the hair composition.

In some embodiments, a treatment with a hair composition of the application promotes a protective effect of alpha-keratin structure with at least 10%, 15%, 20%, 25%, 30%, 35%, 40%, 45%, 50% or more increase in enthalpy assessed through DSC (Differential Scanning Calorimetry) measurements, as compared with the treatment with a control hair composition that does not contain the combination of the first component and second component used in the hair composition.

The methods of treating hair, according to the procedures discussed above, are useful for conditioning the hair. The procedures are also useful in methods for increasing hair strength, prevent hair damages, and repair hairs during or after damages. The procedures are also useful in methods for increasing natural look and feel of the hair, reducing hair frizz, improving hair smoothness, hair alignment, and/or hair shine. Thus, the instant disclosure relates to methods for: (a) conditioning the hair; (b) improving the look and feel of hair; (c) reducing hair frizz or propensity for frizzing; (d) improving hair smoothness, (e) improving hair alignment; (e) improving or increasing hair shine; (f) increasing hair strength; (g) preventing hair damages; (h) repairing hair damages; the method comprising applying a hair treatment composition according to the instant disclosure to the hair.

### Kits

The hair treatment compositions disclosed herein can be provided in a kit. For example, one or more of the hair treatment compositions of the instant disclosure can be included in a kit that optionally also includes one or more additional hair treatment compositions. All of the various compositions in the kit are separately contained. In some instances, the kits include one or more hair treatment compositions according to the instant disclosure and one or more shampoo compositions, wherein each of the one or more hair treatment compositions and the one or more shampoo compositions are separately contained.

### Embodiments

The following list of embodiments is intended to complement, rather than displace or supersede, the previous descriptions.

Embodiment 1. A hair composition comprising:
(1) a first component comprising at least one of a compound of Formula (I) and a compound of Formula (II)

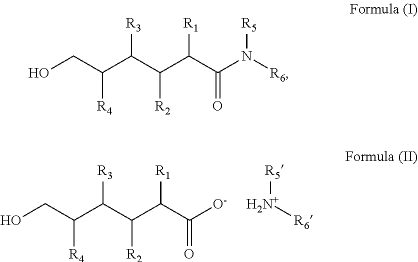

wherein R₁-R₄ and R₁′-R₄′ are independently hydrogen, a hydrocarbon radical having 1 to 10 carbon atoms, a hydroxyl group, an amino group, a sulfhydryl group, an aryl group, or a halogen, and
R₅, R₆, R₅′ and R₆′ are independently hydrogen, an aliphatic hydrocarbon group, an alicyclic hydrocarbon group, an aryl group, an alkylaryl group or a heterocyclic group, with the proviso that R₅′ and R₆′ are not simultaneously hydrogen, wherein the aliphatic hydrocarbon group, the alicyclic hydrocarbon group, the aryl group, the alkylaryl group or the heterocyclic group is substituted with at least one hydroxyl group;
(2) a second component comprising:
   a. an α,β unsaturated dicarboxylic acid and/or an ester thereof;
   b. a nonionic crosslinking agent selected from the group consisting of polyols, saccharides or a combination thereof; and
   c. an ionic cross-linking agent selected from the group consisting of hydrolyzed proteins, amino acids or a combination thereof.

Embodiment 2. The hair composition of Embodiment 1, wherein the first component comprises the compound of Formula (I) and the compound of Formula (II).

Embodiment 3. The hair composition of Embodiment 1 or 2, wherein R₁-R₄ and R₁′-R₄′ are each a hydroxyl group, and R₅, R₆, R₅′ and R₆′ are independently hydrogen, or a —CH₂CH₂CH₂OH group.

Embodiment 4. The hair composition of any one of the Embodiments 1-3, wherein the α,β unsaturated dicarboxylic acid in the second component comprises itaconic acid, acrylic acid, maleic acid, fumaric acid, or methacrylic acid.

Embodiment 5. The hair composition of any one of the Embodiments 1-4, wherein the nonionic crosslinking agent in the second component comprises panthenol or propanediol.

Embodiment 6. The hair composition of any one of the Embodiments 1-5, wherein the ionic cross-linking agent in the second component comprises an amino acid, wherein the amino acid is selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan.

Embodiment 7. The hair composition of any one of the Embodiments 1-6, wherein the second component further comprises one or more plant or seed extracts, preferably selected from the group consisting of Linum usitatissimum (Linseed) Seed Extract, Salvia hispanica (Chia) Seed Extract.

Embodiment 8. The hair composition of any one of the Embodiments 1-7, wherein the second component further comprises an ester, such as an ester of glycerol and a saturated or unsaturated, branched or unbranched carboxylic acid, such as Polyglyceryl-10 Laurate.

Embodiment 9. The hair composition of any one of the Embodiments 1-8, wherein the total amount of the first component is about 0.1% to 5%, such as 0.3% to 3%, 0.75% to 2.5%, 0.5% to 3%, 0.1% to 3%, 0.2% to 2.5%, 0.1% to 2.5%, by weight based on the total weight of the hair composition.

Embodiment 10. The hair composition any one of the Embodiments 1-9, wherein the total amount of the second component is about 0.1% to 5%, such as 0.3% to 3%, 0.2% to 2%, 0.05% to 5%, 0.05% to 2% by weight, based on the total weight of the hair composition.

Embodiment 11. The hair composition of any one of the Embodiments 1-10, further comprising at least one additional component, such as one selected from the group consisting of surfactants, fatty acid soap, hair and skin conditioning agents, suspending aids, emollients, emulsifiers, rheology modifiers, thickening agents, vitamins, hair growth promoters, self-tanning agents, sunscreens, skin lighteners, anti-aging compounds, anti-wrinkle compounds, hair coloring treatment agents, hair color protecting agents, hair styling products, anti-cellulite compounds, anti-acne compounds, anti-dandruff agents, anti-inflammatory compounds, analgesics, antiperspirant agents, deodorant agents, hair fixatives, particulates, abrasives, moisturizers, antioxidants, keratolytic agents, anti-static agents, foam boosters, hydrotropes, solubilizing agents, chelating agents, antimicrobial agents, antifungal agents, pH adjusting agents, chelating agents, buffering agents, botanicals, hair colorants, oxidizing agents, reducing agents, hair and skin bleaching agents, pigments, anticaries, anti-tartar agents, anti-plaque agents, solvents, and combinations thereof.

Embodiment 12. The hair composition of any one of the Embodiments 1-11, comprising one or more preservatives, such as sodium benzoate, phenoxyethanol, benzyl alcohol, ethylhexylglycerin, benzoic acid, xylityl sesquicaprylate, ethylhexylglycerin, caprylic acid, itaconic acid, benzoic acid, potassium sorbate, and combinations thereof.

Embodiment 13. The hair composition of any one of the Embodiments 1-12, comprising a preservative, such as a benzoic acid or salt thereof, xylityl sesquicaprylate, benzyl alcohol, or ethylhexylglycerin.

Embodiment 14. The hair composition of any one of the Embodiments 1-13, comprising one or more surfactants, such as a cationic surfactant, non-ionic surfactant, amphoteric surfactant, or anionic surfactant.

Embodiment 15. The hair composition of any one of the Embodiments 1-14, comprising one or more cationic surfactants, such as one or more cationic surfactants selected from the group consisting of polyquaternium-37, hydroxyethyl cetearamidopropyldimonium chloride, behentrimonium chloride, cetrimonium chloride, ethyl lauroyl arginate HCl, cetyltrimethyl ammonium chloride (CTAC), polyquaternium-10, disunfloweroylethyl dimonium chloride, polyquaternium-73, behenamidopropyl dimethylamine, stearamidopropyl dimethylamine, polyquaternium-16, brassicamidopropyl dimethylamine, polyquaternium-7, Guar hydroxypropyltrimonium chloride, polyquaternium-11, and combinations thereof.

Embodiment 16. The hair composition of any one of the Embodiments 1-15, comprising one or more cationic surfactants, such as one or more cationic surfactants selected from the group consisting of a polyquaternium, such as polyquaternium-10, polyquaternium-7 and polyquaternium-73, Guar hydroxypropyltrimonium chloride, behenamidopropyl dimethylamine, polyquaternium-37, behentrimonium chloride, brassicamidopropyl dimethylamine, stearamidopropyl dimethylamine, polyquaternium-16, disunfloweroylethyl dimonium chloride, cetrimonium chloride, hydroxyethyl cetearamidopropyldimonium chloride, and polyquaternium-11.

Embodiment 17. The hair composition of any one of the Embodiments 1-16, comprising one or more non-ionic surfactants, such as one or more non-ionic surfactants selected from the group consisting of C₁₂₋₂₀ alkyl glucoside, trideceth-7, Trideceth-8, Crambe abyssinica seed oil phytosterol esters, lauryl lactyl lactate, Trideceth-12, lauryl glucoside, Sunfloweroyl methylglucamide, decyl glucoside, coco-glucoside, cocoyl methyl glucamide, capryloyl/caproyl methyl glucamide, lauroyl/myristoyl methyl glucamide, cocamidopropylamine oxide, decylamine oxide, and combinations thereof.

Embodiment 18. The hair composition of any one of the Embodiments 147, comprising one or more non-ionic surfactants, such as one or more non-ionic surfactants selected from the group consisting of lauryl glucoside, Sunfloweroyl methylglucamide.

Embodiment 19. The hair composition of any one of the Embodiments 1-18, comprising one or more amphoteric surfactants, such as one or more amphoteric surfactants selected from the group consisting of betaine, cocamidopropyl hydroxysultaine, lauryl betainec, and combinations thereof.

Embodiment 20. The hair composition of any one of the Embodiments 1-19, comprising one or more amphoteric surfactants, such as one or more amphoteric surfactants selected from the group consisting of cocamidopropyl hydroxysultaine, or lauryl betaine.

Embodiment 21. The hair composition of any one of the Embodiments 1-20, comprising one or more anionic surfactants, such as one or more anionic surfactants selected from the group consisting of sodium lauroyl methyl isethionate, sodium cocoyl glutamate, cocamidopropyl hydroxysultaineutamate, disodium cocoyl glutamate, sodium lauroyl lactylate, sodium methyl cocoyl taurate, sodium lauroyl sarcosinate, and combinations thereof.

Embodiment 22. The hair composition of any one of the Embodiments 1-21, comprising one or more anionic surfactants, such as one or more anionic surfactants selected from the group consisting of sodium lauroyl methyl isethionate, sodium cocoyl glutamate, disodium cocoyl glutamate, sodium lauroyl lactylate, sodium methyl cocoyl taurate, or sodium lauroyl sarcosinate.

Embodiment 23. The hair composition of any one of the Embodiments 1-22, further comprising one or more emollients, such as one or more selected from the group consisting of panthenol, propanediol opernicia Cerifera (Carnauba) Wax, C13-15 alkane, glycol distearate, Cocos nucifera (coconut) oil, avocado oil, glyceryl ricinoleate, Ricinus communis (castor) seed oil, dimethicone, dimethiconol, Argania spinosa kernel oil (Argan oil), ethylhexyl olivate, squalane, gluconamido amodimethicone, glyceryl stearate, Sunflower Seed Oil Glycerides, lauryl lactyl lactate, ethylhexyl palmitate, Olea europaea (olive) fruit unsaponifiables, caprylic/capric triglyceride, ethylhexyl stearate, Crambe abyssinica seed oil phytosterol esters, Mangifera indica (mango) seed butter, castor oil propanediol esters, Helianthus annuus (sunflower) seed oil unsaponifiables, Plukenetia volubilis seed oil, Salvia hispanica seed oil, Astrocaryum vulgare Kernel oil, undecane, tridecane, Simmondsia chinensis (Jojoba) seed oil, Crambe abyssinica (Abyssinian) seed oil, Helianthus annuus (sunflower) seed oil, Astrocaryum murumuru Seed butter, amodimethicone, polycitronellol acetate, cetyl esters Simmondsia chinensis (jojoba) butter, Jojoba Wax PEG-120 esters, acrylates copolymer, Polyquaternium-7, and combinations thereof.

Embodiment 24. The hair composition of any one of the Embodiments 1-23, further comprising one or more emollients, such as one or more selected from the group consisting of Glycol Distearate, Jojoba Wax PEG-120 esters, Dimethicone, Ethylhexyl palmitate, Olea Europaea (olive) fruit unsaponifiables, Caprylic/Capric Triglyceride, Ethylhexyl Stearate, Castor oil propanediol esters, Crambe abyssinica seed oil phytosterol, Helianthus annuus (sunflower) seed oil, Simmondsia chinensis (jojoba) butter, Astrocaryum murumuru Seed butter, Plukenetia volubilis seed oil, Salvia hispanica seed oil, Polycitronellol Acetate, Undecane, Tridecane, Simmondsia chinensis (Jojoba) seed oil, Crambe abyssinica (Abyssinian) Seed oil, Ricinus communis (castor) seed oil, Helianthus annuus (sunflower) seed oil unsaponifiables, Mangifera indica (mango) seed butter, Squalane, Astrocaryum vulgare Kernel oil, Dimethiconol, Argania spinosa kernel oil (Argan oil), Ethylhexyl Olivate, Glyceryl Ricinoleate, C₁₃₋₁₅ Alkane, Cocos nucifera (coconut) oil, Avocado oil, or Gluconamido Amodimethicone.

Embodiment 25. The hair composition of any one of the Embodiments 1-24, further comprising one or more thickening agents, wherein the thickening agents comprise cetyl hydroxyethylcellulose, hydropropylcelluloses, cetearyl alcohol, Copernicia cerifera (Carnauba) wax, behenyl alcohol, Raphanus sativus (Radish) seed extract, Rosmarinus officinales (rosemary) leaf extract, Tetrahexyldecyl Ascorbate, Caesalpina spinosa Fruit extract, Helianthus annuus (sunflower) sprout extract, cetyl alcohol, acrylamidopropyl trimonium chloride/acrylamide copolymer, acrylates copolymer-4, lauryl lactate, hydroxypropyl starch phosphate, acrylates copolymer, ammonium acryloyldimethyltaurate, sodium acryloyldimethyltaurate/VP copolymer, sodium acrylate/sodium acryloyldimethyl taurate copolymer, sodium polyacrylate, polyacrylate crosspolymer-11, carbomer, acrylates/C10-30 alkyl acrylate crosspolymer, acrylates copolymer, Polyquaternium-73, hydroxyethyl acrylate/sodiumacryloyldimethyltaurate copolymer, lithium magnesium sodium silicate, sodium magnesium fluorosilicate, glycol distearate, hydrogenated castor oil, hydrogenated castor oil hydroxystearate, hydrogenated castor oil isostearate, hydrogenated castor oil stearate, hydrogenated castor PEG-8 esters, or combinations thereof.

Embodiment 26. The hair composition of any one of the Embodiments 1-25, further comprising one or more a thickening agent, preferably the thickening agent comprise acrylates copolymer, or Polyquaternium-73.

Embodiment 27. The hair composition of any one of the Embodiments 1-26, further comprising one or more chelating agents, wherein the chelating agents comprise Disodium EDTA, tetrasodium glutamate diacetate, or combinations thereof.

Embodiment 28. The hair composition of any one of the Embodiments 1-27, further comprising one or more humectants, wherein the humectants comprise panthenol, propanediol, glycerin, hydroxyacetophenone, Plukenetia volubilis seed oil, Salvia hispanica seed oil, Undecane, Tridecane, Simmondsia chinensis (Jojoba) seed oil, Crambe abyssinica (Abyssinian) Seed oil, Ricinus communis (castor) seed oil, Helianthus annuus (sunflower) seed oil, 1,2,6 hexanetriol, dipropylene glycol, butylene glycol, caprylyl glycol, pentylene glycol, isopentyldiol, hexylene glycol, phytantriol, propylene glycol, sodium PCA, sorbitol, triethylene glycol, polyglyceryl sorbitol, glucose, fructose, mannitol, xylitol, inositol, trehalose, polydextrose, potassium PCA, hydrogenated honey, hyaluronic acid, inositol, erythritol, capryl glycol, acetamidoethoxyethanol, or combinations thereof.

Embodiment 29. The hair composition of any one of the Embodiments 1-28, further comprising one or more exfoliators, wherein the exfoliators comprise citric acid, Lactobacillus/Pumpkin Fruit Ferment Filtrate, or combinations thereof.

Embodiment 30. The hair composition of any one of the Embodiments 1-29, further comprising one or more natural extracts, wherein the extracts comprise Caesalpina spinosa Fruit extract, Helianthus annuus (sunflower) sprout extract, Plukenetia volubilis seed oil, Salvia hispanica seed oil, Astrocaryum vulgare Kernel oil, Tetrahexyldecyl Ascorbate, Simmondsia chinensis (Jojoba) seed oil, Crambe abyssinica (Abyssinian) Seed oil, Ricinus communis (castor) seed oil, Helianthus annuus (sunflower) seed oil, Raphanus sativus (Radish) seed extract, Rosmarinus officinalis (rosemary) leaf extract, Copernicia cerifera (Carnauba) Wax, or combinations thereof.

Embodiment 31. The hair composition of any one of the Embodiments 1-30, further comprising one or more vitamins, wherein the vitamins comprise tocopherol, panthenol, retinol, retinyl palmitate, ascorbic acid, tocopherol acetate, or combinations thereof.

Embodiment 32. The hair composition of any one of the Embodiments 1-31, further comprising one or more antioxidants, wherein the antioxidants comprise hydroxyacetophenone, Rosmarinus officinalis (rosemary) leaf extract (WS), carnosine, tetrahexyl decyl ascorbate, bioflavonoids, lycopene, or combinations thereof.

Embodiment 33. The hair composition of any one of the Embodiments 1-32, further comprising one or more emulsifiers, wherein the emulsifiers comprise behenyl alcohol, cetearyl alcohol, palmitic acid, stearic acid, C14-22 alcohols, glyceryl stearate, cetyl alcohol, stearyl alcohol, Crambe abyssinica seed oil phytosterol esters, Trideceth-12, caprylic/capric/diglyceryl succinate, stearic acid, C14-16 glycol palmitate, C18-20 glycol isostearate, cocamidopropyl lauryl ether, dipentaerythrityl hydroxystearate, dipentaerythrityl hydroxyisostearate, stearoyl glutamic acid, Polysorbate-20, polyglyceryl-10 laurate, polyglyceryl-6 caprylate, polyglyceryl-3 cocoate, polyglyceryl-4 caprate, polyglyceryl-6 ricinoleate, polyglyceryl-6 caprylate, sodium surfactin, palmitic acid, or combinations thereof.

Embodiment 34. The hair composition of any one of the Embodiments 1-33, further comprising one or more alcohols, wherein the alcohols comprise cetyl alcohol, stearyl alcohol, behenyl alcohol, palmitic acid, stearic acid, benzyl alcohol, propylene glycol, panthenol, propanediol, cetearyl alcohol, isopropanol, stearic acid, lauric acid, palmitic acid, arachidic alcohol, isocetyl alcohol, isostearyl alcohol, lauryl alcohol, myristyl alcohol, C30-50 alcohols, lanolin alcohol, or combinations thereof.

Embodiment 35. The hair composition of any one of the Embodiments 1-34, further comprising one or more amino acids, wherein the amino acids comprise arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, or combinations thereof.

Embodiment 36. The hair composition of any one of the Embodiments 1-35, comprising a chelating agent, e.g., EDTA; a moisturizer, e.g., glycerin; an exfoliator, e.g., Lactobacillus/Pumpkin Fruit Ferment Filtrate; and/or an acid, e.g., citric acid.

Embodiment 37. The hair composition of any one of the Embodiments 1-36, further comprising water.

Embodiment 38. The hair composition of any one of the Embodiments 1-37, further comprising one or more PH adjusters.

Embodiment 39. The hair composition of any one of the Embodiments 1-38, having a pH of 4-6.

Embodiment 40. The hair composition of any one of the Embodiments 1-39, further comprising Betaine, preferably at the amount of 1% to 15% by weight.

Embodiment 41. The hair composition of any one of the Embodiments 1-40, further comprising natural extracts; preferably the extracts comprise one or more of Raphanus sativus (Radish) seed extract, Rosmarinus officinales (rosemary) leaf extract, Tetrahexyldecyl Ascorbate, Caesalpina spinosa Fruit extract, or Helianthus annuus (sunflower) sprout extract.

Embodiment 42. The hair composition of any one of the Embodiments 1-41, wherein the first component contains hydroxypropylgluconamide and hydroxypropylammonium gluconate.

Embodiment 43. The hair composition of Embodiment 42, wherein the first component contains 50% hydroxypropylgluconamide and hydroxypropylammonium gluconate, 49% water, and 1% preservatives (such as benzyl alcohol, sodium benzoate, potassium sorbate) and pH adjuster (such as tartaric acid).

Embodiment 44. The hair composition of any one of the Embodiments 1-43, wherein the first component is FiberHance™ bm.

Embodiment 45. The hair composition of any one of the Embodiments 1-44, wherein the second component contains dicarboxylic acid, amino acid, provitamin B5 and polysaccharides extracted from seeds of Flaxseed (Linum usitatissimum) and Chia (Salvia hispanica.

Embodiment 46. The hair composition of Embodiment 45, wherein the second component contains 30-70% water, 5-20% itaconic acid, 5-20% arginine, 1-15% panthenol, 1-10% Linum usitatissimum (Linseed) seed extract, 1-9% Salvia hispanica seed extract, 1-8% polyglyceryl-10 Laurate, 0.2-0.9% xylitol, and 0.2-0.9% caprylic acid.

Embodiment 47. The hair composition of any one of the Embodiments 1-46, wherein the second component is Restart Pro.

Embodiment 48. The hair composition of Embodiment 47, wherein the first component is FiberHance™ bm and the second component is Restart Pro.

Embodiment 49. The hair composition of any one of the Embodiments 42-48, wherein the total amount of the first component is about 0.1% to 5%, such as 0.3% to 3%, 0.75% to 2.5%, 0.5% to 3%, 0.1% to 3%, 0.2% to 2.5%, 0.1% to 2.5%, by weight based on the total weight of the hair composition.

Embodiment 50. The hair composition any one of the Embodiments 42-49, wherein the total amount of the second component is about 0.1% to 5%, such as 0.3% to 3%, 0.2% to 2%, 0.05% to 5%, 0.05% to 2% by weight, based on the total weight of the hair composition.

Embodiment 51. The hair composition of the Embodiment 50, wherein the total amount of the first component is about 0.1% to 5%, and the total amount of the second component is about 0.1% to 5%; the total amount of the first component is about 0.1% to 3%, and the total amount of the second component is about 0.05% to 2%; or the total amount of the first component is about 0.2% to 2.5%, and the total amount of the second component is about 0.05% to 2%.

Embodiment 52. The hair composition of any one of the Embodiments 1-51, wherein the hair composition is a shampoo, conditioner, mask, or leave-in treatment.

Embodiment 53. The hair composition of any one of the Embodiments 1-52, wherein the hair composition is formulated for treatment of a hair type selected from straight, wavy, curly, coily, or a combination thereof.

Embodiment 54. A method of preparing the hair composition of any one of the Embodiments 1-53, comprising combining the components to thereby obtain the hair composition.

Embodiment 55. A method for treating hair comprising applying to the hair the hair composition of any one of the Embodiments 1-53.

Embodiment 56. A method of strengthening a hair fiber comprising applying to the hair fiber the hair composition of any one of the Embodiments 1-53.

Embodiment 57. A method of reducing and/or preventing hair breakage applying to the hair the hair composition of any one of the Embodiments 1-53.

Embodiment 58. The method of any one of the Embodiments 55-57, wherein the hair or hair fiber is virgin hair, hair treated with a chemical or mechanical hair treatment (e.g., bleached hairs, dyed hairs), or damaged or weakened hair fiber.

### EXAMPLES

The following examples illustrate the presently disclosed and/or claimed inventive concept(s). Each example is provided by way of illustrating the presently disclosed and claimed inventive concept(s), not limitation of the presently disclosed and claimed inventive concept(s). In fact, it will be apparent to those skilled in the art that various modifications and variations can be made in the presently disclosed and claimed inventive concept(s) without departing from the scope or spirit of the invention. For instance, features illustrated or described as part of one embodiment, can be used on another embodiment to yield a still further embodiment. Thus, it is intended that the presently disclosed and claimed inventive concept(s) covers such modifications and variations as come within the scope of the appended claims and their equivalents.

### Example 1: Synergistic Effect Between Restart Pro and Fiberhance BM in Reducing Hair Breakage

Hair samples treated by the essential ingredients in Example 1 were prepared. The hair strength was measured through hair breakage data, using the Sirtaki combing machine. This hair breakage data was compared to hair samples treated by two other separate solutions.

Two solution products were obtained from the commercial sources:
Restart Pro: commercially available from Chemyunion, containing a mixture of water, itaconic acid, Arginine, panthenol, Linum usitatissimum seed extract, Salvia hispanica seed extract, polyglyceryl-10 laurate, xylitol, and caprylic acid.
FiberHance™ bn: commercially available from Ashland, containing a mixture of hydroxypropyl gluconamide, hydroxypropyl ammonium gluconate, water, preservatives and pH adjuster.

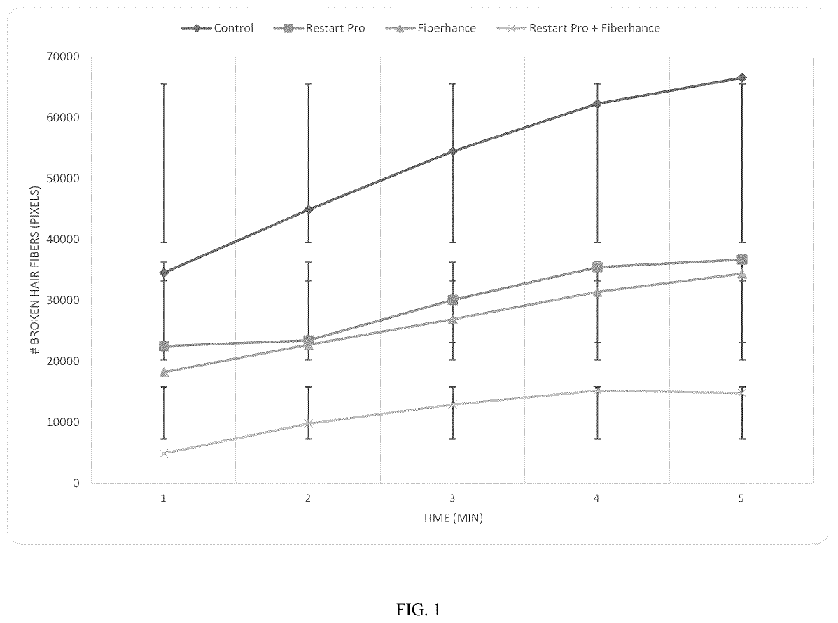

24 hair tresses of the double bleached wavy and curly hair were prepared, each weighing 3-4 g. Each tress was washed with 0.9 g Suave Clarifying Shampoo and blow-dried on low heat. These 24 hair tresses were then divided into 4 groups, each contains 6 tresses. A group of 6 tresses were placed into 400 mL of water (control), a 2% Restart Pro, a 2% FiberHance™ bm, and a combination of 1% Restart Pro+1% FiberHance™ bm respectively. Each tress was taken out after 30 minutes and squeezed 3 times to remove excess water. The tresses were then blow-dried and gently combed to prevent tangling. The tresses were then tested using the Sirtaki combing machine and the hair breakage data of the hair after double bleaching are illustrated in FIG. 1, Table A and Table B, where the indicated time, e.g., 3 minutes, refers to the time the brushing machine was run in the hair breakage performance test. For example, running the brushing machine for 3 minutes also equals to 240 combing cycles for this study.

TABLE A
Comparison of Hair Breakage Performance
Over Time with Different Hair Treatments

| | Improvement | | | | |
|---|---|---|---|---|---|
| | | | Restart | Restart | Restart |
| | | | Pro + | Pro + | Pro + |
| | Restart | Fiberhance | Fiberhance | Fiberhance | Fiberhance |
| | Pro vs. | vs. | vs. | vs. | vs. |
| Time | control | control | control | Restart PRO | Fiberhance |
| 1 min | 1.54 | 1.89 | 7.01 | 4.57 | 3.70 |
| 2 min | 1.91 | 1.97 | 4.58 | 2.40 | 2.32 |
| 3 min | 1.81 | 2.02 | 4.21 | 2.33 | 2.09 |
| 4 min | 1.76 | 1.98 | 4.09 | 2.33 | 2.06 |
| 5 min | 1.81 | 1.93 | 4.49 | 2.48 | 2.32 |

TABLE B
Comparison of Hair Breakage Performance after 3 Minutes

| | Hair Treatment | | | |
|---|---|---|---|---|
| | Control | | 2% FiberHance ™ | 1% Restart Pro + |
| | (no treatment) | 2% Restart Pro | bm | 1% FiberHance ™ bm |
| Hair | No changes in | 1.81x reduction | 2.02x reduction | 4.21x reduction in |
| breakage | hair breakage | in hair breakage | in hair breakage | hair breakage |

The results showed that the combination of Restart Pro and FiberHance™ bm provided significantly more hair protection than the additive effect of Restart Pro and FiberHance™ bm. In particular, a hair composition containing both the ingredients of Restart Pro and FiberHance™ bm increased hair strength significantly than a hair composition containing only Restart Pro or FiberHance™ bm. For example, a hair composition containing 1% Restart Pro and 1% FiberHance™ bm increased hair strength 4.21 times as compared with the control, while a hair composition containing only 2% Restart Pro or 2% FiberHance™ bm increased hair strength only 1.81 times and 2.02 times respectively.

The inventors of the application surprisingly discovered this synergistic effect of Restart Pro and FiberHance™ bm. Without wishing to be bound by theory, it is believed that ingredients in Restart PRO actively crosslink the broken bonds within the cortex, contributing to increased hair strength, while ingredients in FiberHance™ bm penetrate the cortex and create new hydrogen and ionic bonds to support damaged keratin structure, and also provide significant reparative action on the cuticle level.

### Example 2: Preparation of Hair Compositions

Exemplary hair compositions are described in this Example. The exemplary hair compositions can be prepared by conventional formulation and mixing techniques in view of the present disclosure, or by mixing together one or more polyols (e.g., glycerin), water, surfactants, other liquid ingredients, along with any solids that need to be melted at an elevated temperature. The ingredients can be mixed thoroughly at elevated temperature and then cooled to ambient temperature. Additional ingredients are added to the cooled product. The amount stated reflects the weight percent of the active material, unless otherwise specified. The hair compositions include different hair products, such as shampoo for straight and/or wavy hair, shampoo for curly and/or coily hair, conditioner for straight and/or wavy hair, conditioner for curly and/or coily hair, hair mask for straight and/or wavy hair, hair mask for curly and/or coily hair, leave-in treatment product (LIT) for straight and/or wavy hair, and LIT for curly and/or coily hair.

Exemplary Formulation 1

| Percent Formula | |
|---|---|
| (% w/w) | Ingredient |
| 0.1-5 | First component |
| 0.1-5 | Second component |
| 90-99.8 | One or more other ingredients suitable for hair |
| | composition, e.g., water, humectants, surfactants, |
| | emollients and/or thickeners, exfoliators, preservatives, |
| | antifungals and/or antimicrobials, pH adjusters |

Exemplary Formulation 2

| Percent Formula | |
|---|---|
| (% w/w) | Ingredient |
| Adjust to 100 | Water |
| 0.1-0.3 | chelating agent |
| 1-3 | One or more humectants |
| 25-40 | One or more surfactants |
| 2-8 | One or more emollients and/or thickeners |
| 0-0.2 | One or more exfoliators |
| 0.5-2.5 | One or more preservatives, antifungals and/or |
| | antimicrobials |
| 0.3-3 | First component |
| 0.3-3 | Second component |
| 0.3-1 | pH - Adjuster |

Exemplary Formulation 3

| Percent Formula | |
|---|---|
| (% w/w) | Ingredient |
| Adjust to 100 | Water |
| 0.1-0.3 | chelating agent |
| 1-3 | One or more humectant |
| 0-0.5 | One or more cationic surfactants |
| 5-10 | One or more non-ionic surfactants |
| 5-15 | One or more amphoteric surfactants |
| 15-25 | One or more anionic surfactants |
| 2-8 | One or more emollients and/or thickeners |
| 0.1 | Exfoliator |
| 0.5-2.5 | One or more preservatives, antifungals and/or |
| | antimicrobials |
| 0.75-2.5 | First component |
| 0.2-2 | Second component |
| 0.3-1 | pH - Adjuster |

Exemplary Formulation 4

| Percent Formula | |
|---|---|
| (% w/w) | Ingredient |
| Adjust to 100 | Water |
| 0.1-0.3 | Disodium EDTA |
| 1-3 | One or more humectants |
| 0.1-5 | One or more of polyquaternium, such as polyquaternium-10, |
| | polyquaternium-7 and polyquaternium-73, Guar hydroxypropyltrimonium |
| | chloride, behenamidopropyl dimethylamine, polyquaternium-37, |
| | Behentrimonium chloride, Brassicamidopropyl Dimethylamine, |
| | Stearamidopropyl Dimethylamine, Polyquaternium-16, |
| | Disunfloweroylethyl Dimonium Chloride, Cetrimonium Chloride, |
| | Hydroxyethyl Cetearamidopropyldimonium Chloride, and |
| | Polyquaternium-11 |
| 5-10 | One or more of lauryl glucoside, Sunfloweroyl methylglucamide |
| 5-15 | One or more of cocamidopropyl hydroxysultaine, and lauryl betaine |
| 15-25 | One or more of sodium lauroyl methyl isethionate, sodium cocoyl |
| | glutamate, disodium cocoyl glutamate, sodium lauroyl lactylate, sodium |
| | methyl cocoyl taurate, and sodium lauroyl sarcosinate |
| 2-8 | One or more of Glycol Distearate, Jojoba Wax PEG-120 esters, |
| | Dimethicone, Ethylhexyl palmitate, Olea Europaea (olive) fruit |
| | unsaponifiables, Caprylic/Capric Triglyceride, Ethylhexyl Stearate, Castor |
| | oil propanediol esters, Crambe Abyssinica seed oil phytosterol, |
| | Helianthus annuus (sunflower) seed oil, Simmondsia Chinensis (jojoba) |
| | butter, Astrocaryum Murumuru Seed butter, Plukenetia Volubilis seed oil, |
| | Salvia Hispanica seed oil, Polycitronellol Acetate, Undecane, Tridecane, |
| | Simmondsia Chinensis (Jojoba) seed oil, Crambe abyssinica (Abyssinian) |
| | Seed oil, Ricinus communis (castor) seed oil, Helianthus annuus |
| | (sunflower) seed oil unsaponifiables, Mangifera Indica (mango) seed |
| | butter, Squalane, Astrocaryum Vulgare Kernel oil, Dimethiconol, Argania |
| | spinosa kernel oil (Argan oil), Ethylhexyl Olivate, Glyceryl Ricinoleate, |
| | C₁₃-1₅ Alkane, Cocos nucifera (coconut) oil, Avocado oil, or |
| | Gluconamido Amodimethicone, acrylates copolymer, and |
| | Polyquaternium-7 |
| 0.1 | One or more exfoliator, e.g., Lactobacillus/Pumpkin Fruit Ferment Filtrate |
| 0.5-2.5 | One or more of a benzoic acid, Xylityl Sesquicaprylate, sodium benzoate, |
| | potassium sorbate, benzyl alcohol, or ethylhexylglycerin |
| 0.75-2.5 | First component, such as FiberHance ™ bm |
| 0.2-2 | Second component, such as Restart Pro |
| 0.3-1 | pH - Adjuster |

Exemplary Formulation 5 (e.g., shampoo for straight and/or wavy hair)

| Percent Formula | |
|---|---|
| (% w/w) | Ingredient |
| Adjust to 100 | Water |
| 0.1-1 | Disodium EDTA |
| 1-5 | One or more humectants, such as, but not limited to: 1,2,6 hexanetriol, |
| | dipropylene glycol, butylene glycol, caprylyl glycol, pentylene glycol, |
| | isopentyldiol, hexylene glycol, hydroxyacetophenone, panthenol, |
| | phytantriol, propylene glycol, glycerin, propanediol, sodium PCA, |
| | sorbitol, triethylene glycol, polyglyceryl sorbitol, glucose, fructose, |
| | mannitol, xylitol, inositol, trehalose, polydextrose, potassium PCA, |
| | hydrogenated honey, hyaluronic acid, inositol, erythritol, capryl glycol, |
| | and acetamidoethoxyethanol. |
| 0.1-12 | One or more of cationic surfactants, such as, but not limited to: |
| | polyquaternium-37, Behentrimonium chloride, Brassicamidopropyl |
| | Dimethylamine, Stearamidopropyl Dimethylamine, Polyquaternium-16, |
| | polyquaternium-10, polyquaternium-7 and polyquaternium-73, Guar |
| | hydroxypropyltrimonium chloride, behenamidopropyl dimethylamine, |
| | Disunfloweroylethyl Dimonium Chloride, Cetrimonium Chloride, |
| | Hydroxyethyl Cetearamidopropyldimonium Chloride, and |
| | Polyquaternium-11 |
| 5-12 | One or more of non-ionic surfactants, such as, but not limited to: lauryl |
| | glucoside, and Sunfloweroyl methylglucamide |
| 5-20 | One or more of amphoteric surfactants, such as, but not limited to: |
| | cocamidopropyl hydroxysultaine, and lauryl betaine |
| 10-30 | One or more of anionic surfactants, such as, but not limited to: sodium |
| | lauroyl methyl isethionate, sodium cocoyl glutamate, Cocamidopropyl |
| | Hydroxysultaineutamate, disodium cocoyl glutamate, sodium lauroyl |
| | lactylate, sodium methyl cocoyl taurate, and sodium lauroyl sarcosinate |
| 0.5-12 | One or more of emollients, such as, but not limited to: Dimethicone, |
| | Ethylhexyl palmitate, Olea Europaea (olive) fruit unsaponifiables, |
| | Caprylic/Capric Triglyceride, Glycol Distearate, Jojoba Wax PEG-120 |
| | esters, Ethylhexyl Stearate, Castor oil propanediol esters, Crambe |
| | Abyssinica seed oil phytosterol, Helianthus annuus (sunflower) seed oil, |
| | Simmondsia Chinensis (jojoba) butter, Astrocaryum Murumuru Seed |
| | butter, Plukenetia Volubilis seed oil, Salvia Hispanica seed oil, |
| | Polycitronellol Acetate, Undecane, Tridecane, Simmondsia Chinensis |
| | (Jojoba) seed oil, Crambe abyssinica (Abyssinian) Seed oil, Ricinus |
| | communis (castor) seed oil, Helianthus annuus (sunflower) seed oil |
| | unsaponifiables, Mangifera Indica (mango) seed butter, Squalane, |
| | Astrocaryum Vulgare Kernel oil, Dimethiconol, Argania spinosa kernel |
| | oil (Argan oil), Ethylhexyl Olivate, Glyceryl Ricinoleate, C₁₃-1₅ Alkane, |
| | Cocos nucifera (coconut) oil, Avocado oil, or Gluconamido |
| | Amodimethicone, acrylates copolymer, and Polyquaternium-7 |
| 0.05-2 | One or more exfoliators, such as, but not limited: Lactobacillus/Pumpkin |
| | Fruit Ferment Filtrate, and citric acid |
| 1-10 | One or more thickening agents, such as, but not limited: acrylates |
| | copolymer-4, acrylamidopropyl trimonium chloride/acrylamide |
| | copolymer, and Polyquaternium-73 |
| 0.25-5 | One or more of preservatives, such as, but not limited: benzoic acid, |
| | Xylityl Sesquicaprylate, sodium benzoate, potassium sorbate,benzyl |
| | alcohol, and ethylhexylglycerin |
| 0.1-2.5 | First component, such as FiberHance ™ bm |
| 0.05-2 | Second component, such as Restart Pro |
| 0.2-5 | pH - Adjuster |

Exemplary Formulation 6 (e.g., shampoo for curly and/or coily hair)

| Percent Formula | |
|---|---|
| (% w/w) | Ingredient |
| Adjust to 100 | Water |
| 0.1-1 | Disodium EDTA |
| 1-5 | One or more humectants, such as, but not limited to: 1,2,6 hexanetriol, |
| | dipropylene glycol, butylene glycol, caprylyl glycol, pentylene glycol, |
| | isopentyldiol, hexylene glycol, glycerin, hydroxyacetophenone, |
| | panthenol, phytantriol, propylene glycol, propanediol, sodium PCA, |
| | sorbitol, triethylene glycol, polyglyceryl sorbitol, glucose, fructose, |
| | mannitol, xylitol, inositol, trehalose, polydextrose, potassium PCA, |
| | hydrogenated honey, hyaluronic acid, inositol, erythritol, capryl glycol, |
| | and acetamidoethoxyethanol. |
| 0.1-7 | One or more of cationic surfactants, such as, but not limited to: |
| | polyquaternium-37, Behentrimonium chloride, Brassicamidopropyl |
| | Dimethylamine, Stearamidopropyl Dimethylamine, Polyquaternium-16, |
| | polyquaternium-10, polyquaternium-7 and polyquaternium-73, Guar |
| | hydroxypropyltrimonium chloride, behenamidopropyl dimethylamine, |
| | Disunfloweroylethyl Dimonium Chloride, Cetrimonium Chloride, |
| | Hydroxyethyl Cetearamidopropyldimonium Chloride, and |
| | Polyquaternium-11 |
| 5-12 | One or more of non-ionic surfactants, such as, but not limited to: decyl |
| | glucoside, coco-glucoside, cocoyl methyl glucamide, lauryl glucoside, |
| | Sunfloweroyl methylglucamide, capryloyl/caproyl methyl glucamide, |
| | lauroyl/myristoyl methyl glucamide, cocamidopropylamine oxide, and |
| | decylamine oxide |
| 5-20 | One or more of amphoteric surfactants, such as, but not limited to: |
| | cocamidopropyl hydroxysultaine, and lauryl betaine |
| 10-30 | One or more of anionic surfactants, such as, but not limited to: sodium |
| | lauroyl methyl isethionate, sodium cocoyl glutamate, Cocamidopropyl |
| | Hydroxysultaineutamate, disodium cocoyl glutamate, sodium lauroyl |
| | lactylate, sodium methyl cocoyl taurate, and sodium lauroyl sarcosinate |
| 0.5-12 | One or more of emollients, such as, but not limited to: Glycol Distearate, |
| | Jojoba Wax PEG-120 esters, Dimethicone, Ethylhexyl palmitate, Olea |
| | Europaea (olive) fruit unsaponifiables, Caprylic/Capric Triglyceride, |
| | Ethylhexyl Stearate, Castor oil propanediol esters, Crambe Abyssinica |
| | seed oil phytosterol, Helianthus annuus (sunflower) seed oil, Simmondsia |
| | Chinensis (jojoba) butter, Astrocaryum Murumuru Seed butter, Plukenetia |
| | Volubilis seed oil, Salvia Hispanica seed oil, Polycitronellol Acetate, |
| | Undecane, Tridecane, Simmondsia Chinensis (Jojoba) seed oil, Crambe |
| | abyssinica (Abyssinian) Seed oil, Ricinus communis (castor) seed oil, |
| | Helianthus annuus (sunflower) seed oil unsaponifiables, Mangifera Indica |
| | (mango) seed butter, Squalane, Astrocaryum Vulgare Kernel oil, |
| | Dimethiconol, Argania spinosa kernel oil (Argan oil), Ethylhexyl Olivate, |
| | Glyceryl Ricinoleate, C₁₃-1₅ Alkane, Cocos nucifera (coconut) oil, |
| | Avocado oil, or Gluconamido Amodimethicone, acrylates copolymer, |
| | and Polyquaternium-7 |
| 0.05-2 | One or more exfoliators, such as, but not limited: Lactobacillus/Pumpkin |
| | Fruit Ferment Filtrate, and citric acid |
| 1-10 | One or more thickening agents, such as, but not limited: acrylates |
| | copolymer-4, acrylamidopropyl trimonium chloride/acrylamide |
| | copolymer, and Polyquaternium-73 |
| 0.25-5 | One or more of preservatives, such as, but not limited: benzoic acid, |
| | Xylityl Sesquicaprylate, sodium benzoate, potassium sorbate, benzyl |
| | alcohol, and ethylhexylglycerin |
| 0.1-2.5 | First component, such as FiberHance ™ bm |
| 0.05-2 | Second component, such as Restart Pro |
| 0.2-5 | pH - Adjuster |

Exemplary Formulation 7 (e.g., hair conditioner for straight and/or wavy hair)

| Percent Formula | |
|---|---|
| (% w/w) | Ingredient |
| Adjust to 100 | Water |
| 0.05-1 | Disodium EDTA |
| 1-5 | One or more humectants, such as, but not limited to: 1,2,6 hexanetriol, |
| | dipropylene glycol, butylene glycol, caprylyl glycol, pentylene glycol, |
| | isopentyldiol, hexylene glycol, hydroxyacetophenone, panthenol, |
| | phytantriol, propylene glycol, propanediol, sodium PCA, sorbitol, |
| | triethylene glycol, glycerin, polyglyceryl sorbitol, glucose, fructose, |
| | mannitol, xylitol, inositol, trehalose, polydextrose, potassium PCA, |
| | hydrogenated honey, hyaluronic acid, inositol, erythritol, capryl glycol, |
| | acetamidoethoxyethanol. |
| 0.1-9 | One or more of cationic surfactants, such as, but not limited to: |
| | behenamidopropyl dimethylamine, Stearamidopropyl Dimethylamine, |
| | polyquaternium-73, polyquaternium-10, polyquaternium-7, Guar |
| | hydroxypropyltrimonium chloride, polyquaternium-37, Polyquaternium- |
| | 16, Behentrimonium chloride, Brassicamidopropyl Dimethylamine, |
| | polyquaternium-73, Disunfloweroylethyl Dimonium Chloride, |
| | Cetrimonium Chloride, Hydroxyethyl Cetearamidopropyldimonium |
| | Chloride, and Polyquaternium-11 |
| 0.05-12 | One or more of non-ionic surfactants, such as, but not limited to: crambe |
| | abyssinica seed oil phytosterol esters, lauryl glucoside, Sunfloweroyl |
| | methylglucamide, decyl glucoside, coco-glucoside, cocoyl methyl |
| | glucamide, capryloyl/caproyl methyl glucamide, lauroyl/myristoyl methyl |
| | glucamide, cocamidopropylamine oxide, and decylamine oxide |
| 1-20 | One or more of amphoteric surfactants, such as, but not limited to: |
| | cocamidopropyl hydroxysultaine, betaine, and lauryl betaine |
| 0.25-18 | One or more of emollients, such as, but not limited to: Astrocaryum |
| | Murumuru Seed butter, Dimethicone, cetyl esters, Ethylhexyl palmitate, |
| | Olea Europaea (olive) fruit unsaponifiables, Caprylic/Capric Triglyceride, |
| | Ethylhexyl Stearate, Castor oil propanediol esters, Glycol Distearate, |
| | Jojoba Wax PEG-120 esters, Crambe Abyssinica seed oil phytosterol, |
| | Helianthus annuus (sunflower) seed oil, Simmondsia Chinensis (jojoba) |
| | butter, Plukenetia Volubilis seed oil, Salvia Hispanica seed oil, |
| | Polycitronellol Acetate, Undecane, Tridecane, Simmondsia Chinensis |
| | (Jojoba) seed oil, Crambe abyssinica (Abyssinian) Seed oil, Ricinus |
| | communis (castor) seed oil, Helianthus annuus (sunflower) seed oil |
| | unsaponifiables, Mangifera Indica (mango) seed butter, Squalane, |
| | Astrocaryum Vulgare Kernel oil, Dimethiconol, Argania spinosa kernel |
| | oil (Argan oil), Ethylhexyl Olivate, Glyceryl Ricinoleate, C₁₃-1₅ Alkane, |
| | Cocos nucifera (coconut) oil, Avocado oil, or Gluconamido |
| | Amodimethicone, acrylates copolymer, and Polyquaternium-7 |
| 0.05-2 | One or more exfoliators, such as, but not limited: citric acid, |
| | Lactobacillus/Pumpkin Fruit Ferment Filtrate |
| 1-12 | One or more thickeners, such as, but not limited: cetyl |
| | hydroxyethylcellulose, hydropropylcelluloses, cetyl alcohol, cetearyl |
| | alcohol, lauryl lactate, hydroxypropyl starch phosphate, acrylates |
| | copolymer, ammonium acryloyldimethyltaurate, sodium |
| | acryloyldimethyltaurate/VP copolymer, sodium acrylate/sodium |
| | acryloyldimethyl taurate copolymer, sodium polyacrylate, polyacrylate |
| | crosspolymer-11, carbomer, acrylates/C10-30 alkyl acrylate |
| | crosspolymer, acrylates copolymer, acrylamidopropyl trimonium |
| | chloride/acrylamide copolymer, Polyquaternium-73, hydroxyethyl |
| | acrylate/sodiumacryloyldimethyltaurate copolymer, lithium magnesium |
| | sodium silicate, sodium magnesium fluorosilicate, glycol distearate, |
| | hydrogenated castor oil, hydrogenated castor oil hydroxystearate, |
| | hydrogenated castor oil isostearate, hydrogenated castor oil stearate, |
| | hydrogenated castor PEG-8 esters, Copernicia Cerifera (Carnauba) wax |
| 0.25-5 | One or more of preservatives, such as, but not limited: benzyl alcohol, and |
| | ethylhexylglycerin, benzoic acid, Xylityl Sesquicaprylate, sodium |
| | benzoate, potassium sorbate |
| 0.25-5 | One or more of extracts, such as, but not limited: Raphanus Sativus |
| | (Radish) seed extract, Rosmarinus officinalis (rosemary) leaf extract, |
| | Helianthus Annuus (sunflower) see oil, Tetrahexyldecyl Ascorbate, |
| | Caesalpina Spinosa Fruit extract, Copernicia Cerifera (Carnauba) Wax, or |
| | Helianthus Annuus (sunflower) sprout extract |
| 0.05-5 | One or more of vitamins, such as, but not limited: tocopherol, retinol, |
| | retinyl palmitate, ascorbic acid, tocopherol acetate, and panthenol |
| 0.05-5 | One or more of antioxidants, such as, but not limited: Rosmarinus |
| | officinalis (rosemary) leaf extract (WS), carnosine, tetrahexyl decyl |
| | ascorbate, bioflavonoids, lycopene, daucus carota sativa root cell culture |
| | lysate, monoammonium glycyrrhizinate, alpha lipoic acid (thioctic acid), |
| | Leontopodium alpinum (edelweiss) extract, Vitis vinifera (grape) seed |
| | extract, Camellia sinensis leaf extract, Quercus robur wood extract, |
| | hydrolyzed olive fruit, Olea europea (olive) leaf extract, oleuropin, |
| | hydroxyacetophenone, Punica granatum bark/fruit extract, punicalagin, |
| | ellagic acid, polyphenols, epigallocatechin (EGCG), tannins, |
| | sulforaphane, resveratrol, nordihydroguaiaretic acid |
| 0.1-2.5 | First component, such as FiberHance ™ bm |
| 0.05-2 | Second component, such as Restart Pro |
| 0.2-5 | pH - Adjuster |

Exemplary Formulation 8 (e.g., hair conditioner for curly and/or coily hair)

| Percent Formula | |
|---|---|
| (% w/w) | Ingredient |
| Adjust to 100 | Water |
| 0.05-1 | Disodium EDTA |
| 1-10 | One or more humectants, such as, but not limited to: glycerin, panthenol, |
| | 1,2,6 hexanetriol, dipropylene glycol, butylene glycol, caprylyl glycol, |
| | pentylene glycol, isopentyldiol, hexylene glycol, hydroxyacetophenone, |
| | phytantriol, propylene glycol, propanediol, sodium PCA, sorbitol, |
| | triethylene glycol, polyglyceryl sorbitol, glucose, fructose, mannitol, |
| | xylitol, inositol, trehalose, polydextrose, potassium PCA, hydrogenated |
| | honey, hyaluronic acid, inositol, erythritol, capryl glycol, |
| | acetamidoethoxyethanol. |
| 0.1-9 | One or more of cationic surfactants, such as, but not limited to: |
| | Behentrimonium chloride, Brassicamidopropyl Dimethylamine, |
| | behenamidopropyl dimethylamine, Stearamidopropyl Dimethylamine, |
| | polyquaternium-73, polyquaternium-10, polyquaternium-7, Guar |
| | hydroxypropyltrimonium chloride, polyquaternium-37, Polyquaternium- |
| | 16, Disunfloweroylethyl Dimonium Chloride, Cetrimonium Chloride, |
| | Hydroxyethyl Cetearamidopropyldimonium Chloride, and |
| | Polyquaternium-11 |
| 0.05-12 | One or more of non-ionic surfactants, such as, but not limited to: crambe |
| | abyssinica seed oil phytosterol esters, lauryl glucoside, Sunfloweroyl |
| | methylglucamide, decyl glucoside, coco-glucoside, cocoyl methyl |
| | glucamide, capryloyl/caproyl methyl glucamide, lauroyl/myristoyl methyl |
| | glucamide, cocamidopropylamine oxide, and decylamine oxide |
| 0.25-18 | One or more of emollients, such as, but not limited to: Glycol Distearate, |
| | Jojoba Wax PEG-120 esters, Crambe Abyssinica seed oil phytosterol |
| | esters, Polycitronellol Acetate, Undecane, Tridecane, Simmondsia |
| | Chinensis (Jojoba) seed oil, Crambe abyssinica (Abyssinian) Seed oil, |
| | Ricinus communis (castor) seed oil, Astrocaryum Murumuru Seed butter, |
| | Dimethicone, cetyl esters, Ethylhexyl palmitate, Olea Europaea (olive) |
| | fruit unsaponifiables, Caprylic/Capric Triglyceride, Ethylhexyl Stearate, |
| | Castor oil propanediol esters, Helianthus annuus (sunflower) seed oil, |
| | Simmondsia Chinensis (jojoba) butter, Plukenetia Volubilis seed oil, |
| | Salvia Hispanica seed oil, Helianthus annuus (sunflower) seed oil |
| | unsaponifiables, Mangifera Indica (mango) seed butter, Squalane, |
| | Astrocaryum Vulgare Kernel oil, Dimethiconol, Argania spinosa kernel |
| | oil (Argan oil), Ethylhexyl Olivate, Glyceryl Ricinoleate, C₁₃-1₅ Alkane, |
| | Cocos nucifera (coconut) oil, Avocado oil, or Gluconamido |
| | Amodimethicone, acrylates copolymer, and Polyquaternium-7 |
| 0.05-2 | One or more exfoliators, such as, but not limited: citric acid, |
| | Lactobacillus/Pumpkin Fruit Ferment Filtrate |
| 1-12 | One or more thickeners, such as, but not limited: cetyl |
| | hydroxyethylcellulose, hydropropylcelluloses, cetyl alcohol, cetearyl |
| | alcohol, lauryl lactate, hydroxypropyl starch phosphate, acrylates |
| | copolymer, ammonium acryloyldimethyltaurate, sodium |
| | acryloyldimethyltaurate/VP copolymer, sodium acrylate/sodium |
| | acryloyldimethyl taurate copolymer, sodium polyacrylate, polyacrylate |
| | crosspolymer-11, carbomer, acrylates/C10-30 alkyl acrylate |
| | crosspolymer, acrylates copolymer, acrylamidopropyl trimonium |
| | chloride/acrylamide copolymer, Polyquaternium-73, hydroxyethyl |
| | acrylate/sodiumacryloyldimethyltaurate copolymer, lithium magnesium |
| | sodium silicate, sodium magnesium fluorosilicate, glycol distearate, |
| | hydrogenated castor oil, hydrogenated castor oil hydroxystearate, |
| | hydrogenated castor oil isostearate, hydrogenated castor oil stearate, |
| | hydrogenated castor PEG-8 esters, and Copernicia Cerifera (Carnauba) |
| | wax |
| 0.25-5 | One or more of preservatives, such as, but not limited: benzyl alcohol, and |
| | ethylhexylglycerin, benzoic acid, Xylityl Sesquicaprylate, sodium |
| | benzoate, and potassium sorbate |
| 0.25-5 | One or more of extracts, such as, but not limited: Raphanus Sativus |
| | (Radish) seed extract, Rosmarinus officinalis (rosemary) leaf extract, |
| | Helianthus Annuus (sunflower) see oil, Tetrahexyldecyl Ascorbate, |
| | Caesalpina Spinosa Fruit extract, Copernicia Cerifera (Carnauba) Wax, or |
| | Helianthus Annuus (sunflower) sprout extract |
| 0.05-5 | One or more of vitamins, such as, but not limited: tocopherol, retinol, |
| | retinyl palmitate, ascorbic acid, tocopherol acetate, and panthenol |
| 0.05-5 | One or more of antioxidants, such as, but not limited: Rosmarinus |
| | officinalis (rosemary) leaf extract (WS), carnosine, tetrahexyl decyl |
| | ascorbate, bioflavonoids, lycopene, daucus carota sativa root cell culture |
| | lysate, monoammonium glycyrrhizinate, alpha lipoic acid (thioctic acid), |
| | Leontopodium alpinum (edelweiss) extract, Vitis vinifera (grape) seed |
| | extract, Camellia sinensis leaf extract, Quercus robur wood extract, |
| | hydrolyzed olive fruit, Olea europea (olive) leaf extract, oleuropin, |
| | hydroxyacetophenone, Punica granatum bark/fruit extract, punicalagin, |
| | ellagic acid, polyphenols, epigallocatechin (EGCG), tannins, |
| | sulforaphane, resveratrol, nordihydroguaiaretic acid |
| 0.1-2.5 | First component, such as FiberHance ™ bm |
| 0.05-2 | Second component, such as Restart Pro |
| 0.2-5 | pH - Adjuster |

Exemplary Formulation 9 (e.g., hair mask for straight and/or wavy hair)

| Percent Formula | |
|---|---|
| (% w/w) | Ingredient |
| Adjust to 100 | Water |
| 0.05-1 | Disodium EDTA |
| 1-10 | One or more humectants, such as, but not limited to: glycerin, |
| | hydroxyacetophenone, Undecane, Tridecane, Simmondsia Chinensis |
| | (Jojoba) seed oil, Crambe abyssinica (Abyssinian) Seed oil, Ricinus |
| | communis (castor) seed oil, Helianthus Annuus (sunflower) seed oil, |
| | panthenol, 1,2,6 hexanetriol, dipropylene glycol, butylene glycol, caprylyl |
| | glycol, pentylene glycol, isopentyldiol, hexylene glycol, phytantriol, |
| | propylene glycol, propanediol, sodium PCA, sorbitol, triethylene glycol, |
| | polyglyceryl sorbitol, glucose, fructose, mannitol, xylitol, inositol, |
| | trehalose, polydextrose, potassium PCA, hydrogenated honey, hyaluronic |
| | acid, inositol, erythritol, capryl glycol, and acetamidoethoxyethanol. |
| 0.1-20 | One or more of cationic surfactants, such as, but not limited to: |
| | polyquaternium-73, Polyquaternium-16, Disunfloweroylethyl Dimonium |
| | Chloride, Brassicamidopropyl Dimethylamine, behenamidopropyl |
| | dimethylamine, Stearamidopropyl Dimethylamine, polyquaternium-10, |
| | polyquaternium-7, Guar hydroxypropyltrimonium chloride, |
| | polyquaternium-37, Hydroxyethyl Cetearamidopropyldimonium Chloride, |
| | and Polyquaternium-11, Behentrimonium chloride, Cetrimonium Chloride |
| 0.05-12 | One or more of non-ionic surfactants, such as, but not limited to: crambe |
| | abyssinica seed oil phytosterol esters, lauryl lactyl lactate, Trideceth-12, |
| | lauryl glucoside, Sunfloweroyl methylglucamide, decyl glucoside, coco- |
| | glucoside, cocoyl methyl glucamide, capryloyl/caproyl methyl glucamide, |
| | lauroyl/myristoyl methyl glucamide, cocamidopropylamine oxide, and |
| | decylamine oxide |
| 1-20 | One or more of amphoteric surfactants, such as, but not limited to: betaine, |
| | cocamidopropyl hydroxysultaine, and lauryl betaine |
| 0.25-25 | One or more of emollients, such as, but not limited to: Dimethicone, |
| | Undecane, Tridecane, Simmondsia Chinensis (Jojoba) seed oil, Crambe |
| | abyssinica (Abyssinian) Seed oil, Ricinus communis (castor) seed oil, |
| | Helianthus Annuus (sunflower) seed oil, Ethylhexyl palmitate, Olea |
| | Europaea (olive) fruit unsaponifiables, Caprylic/Capric Triglyceride, |
| | Ethylhexyl Stearate, Helianthus annuus (sunflower) seed oil |
| | unsaponifiables, Crambe Abyssinica seed oil phytosterol esters, Sunflower |
| | Seed Oil Glycerides, lauryl lactyl lactate, Mangifera Indica (mango) seed |
| | butter, Astrocaryum Murumuru Seed butter, Amodimethicone, |
| | Polycitronellol Acetate, cetyl esters, Castor oil propanediol esters, |
| | Simmondsia Chinensis (jojoba) butter, Plukenetia Volubilis seed oil, Salvia |
| | Hispanica seed oil, Glycol Distearate, Jojoba Wax PEG-120 esters, |
| | Mangifera Indica (mango) seed butter, Squalane, Astrocaryum Vulgare |
| | Kernel oil, Dimethiconol, Argania spinosa kernel oil (Argan oil), |
| | Ethylhexyl Olivate, Glyceryl Ricinoleate, C₁₃-1₅ Alkane, Cocos nucifera |
| | (coconut) oil, Avocado oil, Gluconamido Amodimethicone, acrylates |
| | copolymer, and Polyquaternium-7 |
| 0.01-2 | One or more exfoliators, such as, but not limited: citric acid, |
| | Lactobacillus/Pumpkin Fruit Ferment Filtrate |
| 1-12 | One or more thickeners, such as, but not limited: acrylamidopropyl |
| | trimonium chloride/acrylamide copolymer, cetyl hydroxyethylcellulose, |
| | hydropropylcelluloses, cetyl alcohol, cetearyl alcohol, lauryl lactate, |
| | hydroxypropyl starch phosphate, acrylates copolymer, ammonium |
| | acryloyldimethyltaurate, sodium acryloyldimethyltaurate/VP copolymer, |
| | sodium acrylate/sodium acryloyldimethyl taurate copolymer, sodium |
| | polyacrylate, polyacrylate crosspolymer-11, carbomer, acrylates/C10-30 |
| | alkyl acrylate crosspolymer, acrylates copolymer, Polyquaternium-73, |
| | hydroxyethyl acrylate/sodiumacryloyldimethyltaurate copolymer, lithium |
| | magnesium sodium silicate, sodium magnesium fluorosilicate, glycol |
| | distearate, hydrogenated castor oil, hydrogenated castor oil |
| | hydroxystearate, hydrogenated castor oil isostearate, hydrogenated castor |
| | oil stearate, hydrogenated castor PEG-8 esters, and Copernicia Cerifera |
| | (Carnauba) wax |
| 0.05-15 | One or more of emulsifiers, such as, but not limited: stearyl alcohol, |
| | crambe abyssinica seed oil phytosterol esters, Trideceth-12, |
| | caprylic/capric/diglyceryl succinate, C14-16 glycol palmitate, C18-20 |
| | glycol isostearate, cocamidopropyl lauryl ether, dipentaerythrityl |
| | hydroxystearate, dipentaerythrityl hydroxyisostearate, stearoyl glutamic |
| | acid, Polysorbate-20, polyglyceryl-10 laurate, polyglyceryl-6 caprylate, |
| | polyglyceryl-3 cocoate, polyglyceryl-4 caprate, polyglyceryl-6 ricinoleate, |
| | polyglyceryl-6 caprylate, sodium surfactin, C14-22 alcohols, palmitic acid, |
| | stearic acid. |
| 0.05-12 | One or more of alcohols, such as, but not limited: cetearyl alcohol, cetyl |
| | alcohol, stearyl alcohol, isopropanol, benzyl alcohol, propylene glycol, |
| | lauric acid, palmitic acid, stearic acid, arachidic alcohol, behenyl alcohol, |
| | isocetyl alcohol, isostearyl alcohol, lauryl alcohol, myristyl alcohol, C30- |
| | 50 alcohols, and lanolin alcohol |
| 0.05-5 | One or more of preservatives, such as, but not limited: benzyl alcohol, and |
| | ethylhexylglycerin, benzoic acid, Xylityl Sesquicaprylate, sodium |
| | benzoate, and potassium sorbate |
| 0.25-8 | One or more of extracts, such as, but not limited: Simmondsia Chinensis |
| | (Jojoba) seed oil, Crambe abyssinica (Abyssinian) Seed oil, Ricinus |
| | communis (castor) seed oil, Helianthus Annuus (sunflower) seed oil, |
| | Tetrahexyldecyl Ascorbate, Raphanus Sativus (Radish) seed extract, |
| | Rosmarinus officinalis (rosemary) leaf extract, Caesalpina Spinosa Fruit |
| | extract, Copernicia Cerifera (Carnauba) Wax, or Helianthus Annuus |
| | (sunflower) sprout extract |
| 0.05-5 | One or more of vitamins, such as, but not limited: tocopherol, retinol, |
| | retinyl palmitate, ascorbic acid, tocopherol acetate, and panthenol |
| 0.05-5 | One or more of antioxidants, such as, but not limited: Rosmarinus |
| | officinalis (rosemary) leaf extract (WS), carnosine, tetrahexyl decyl |
| | ascorbate, bioflavonoids, lycopene, daucus carota sativa root cell culture |
| | lysate, monoammonium glycyrrhizinate, alpha lipoic acid (thioctic acid), |
| | Leontopodium alpinum (edelweiss) extract, Vitis vinifera (grape) seed |
| | extract, Camellia sinensis leaf extract, Quercus robur wood extract, |
| | hydrolyzed olive fruit, Olea europea (olive) leaf extract, oleuropin, |
| | hydroxyacetophenone, Punica granatum bark/fruit extract, punicalagin, |
| | ellagic acid, polyphenols, epigallocatechin (EGCG), tannins, sulforaphane, |
| | hydroxyacetophenone, resveratrol, nordihydroguaiaretic acid |
| 0.1-2.5 | First component, such as FiberHance ™ bm |
| 0.05-2 | Second component, such as Restart Pro |
| 0.01-5 | pH - Adjuster |

Exemplary Formulation 10 (e.g., hair mask for curly and/or coily hair)

| Percent Formula | |
|---|---|
| (% w/w) | Ingredient |
| Adjust to 100 | Water |
| 0.05-1 | Disodium EDTA |
| 1-10 | One or more humectants, such as, but not limited to: Undecane, Tridecane, |
| | Simmondsia Chinensis (Jojoba) seed oil, Crambe abyssinica (Abyssinian) |
| | Seed oil, Ricinus communis (castor) seed oil, Helianthus Annuus |
| | (sunflower) seed oil, panthenol, 1,2,6 hexanetriol, dipropylene glycol, |
| | butylene glycol, caprylyl glycol, pentylene glycol, isopentyldiol, hexylene |
| | glycol, phytantriol, propylene glycol, propanediol, sodium PCA, sorbitol, |
| | glycerin, hydroxyacetophenone, Plukenetia Volubilis seed oil, Salvia |
| | Hispanica seed oil, triethylene glycol, polyglyceryl sorbitol, glucose, |
| | fructose, mannitol, xylitol, inositol, trehalose, polydextrose, potassium |
| | PCA, hydrogenated honey, hyaluronic acid, inositol, erythritol, capryl |
| | glycol, and acetamidoethoxyethanol. |
| 0.1-20 | One or more of cationic surfactants, such as, but not limited to: |
| | cetyltrimethyl ammonium chloride (CTAC), polyquaternium-10, |
| | Disunfloweroylethyl Dimonium Chloride, polyquaternium-73, |
| | Hydroxyethyl Cetearamidopropyldimonium Chloride, behenamidopropyl |
| | dimethylamine, Stearamidopropyl Dimethylamine, Polyquaternium-16, |
| | Behentrimonium chloride, Cetrimonium Chloride, Brassicamidopropyl |
| | Dimethylamine, polyquaternium-7, Guar hydroxypropyltrimonium |
| | chloride, polyquaternium-37, and Polyquaternium-11 |
| 0.05-12 | One or more of non-ionic surfactants, such as, but not limited to: crambe |
| | abyssinica seed oil phytosterol esters, lauryl lactyl lactate, Trideceth-12, |
| | lauryl glucoside, Sunfloweroyl methylglucamide, decyl glucoside, coco- |
| | glucoside, cocoyl methyl glucamide, capryloyl/caproyl methyl glucamide, |
| | lauroyl/myristoyl methyl glucamide, cocamidopropylamine oxide, and |
| | decylamine oxide |
| 1-10 | One or more of amphoteric surfactants, such as, but not limited to: betaine, |
| | cocamidopropyl hydroxysultaine, and lauryl betaine |
| 0.25-25 | One or more of emollients, such as, but not limited to: Sunflower Seed Oil |
| | Glycerides, lauryl lactyl lactate, Ethylhexyl palmitate, Olea Europaea |
| | (olive) fruit unsaponifiables, Caprylic/Capric Triglyceride, Ethylhexyl |
| | Stearate, Crambe Abyssinica seed oil phytosterol esters, Mangifera Indica |
| | (mango) seed butter, Squalane, Castor oil propanediol esters, Helianthus |
| | annuus (sunflower) seed oil unsaponifiables, Plukenetia Volubilis seed oil, |
| | Salvia Hispanica seed oil, Astrocaryum Vulgare Kernel oil, Dimethicone, |
| | Undecane, Tridecane, Simmondsia Chinensis (Jojoba) seed oil, Crambe |
| | abyssinica (Abyssinian) Seed oil, Ricinus communis (castor) seed oil, |
| | Helianthus Annuus (sunflower) seed oil, Astrocaryum Murumuru Seed |
| | butter, Amodimethicone, Polycitronellol Acetate, cetyl esters Simmondsia |
| | Chinensis (jojoba) butter, Glycol Distearate, Jojoba Wax PEG-120 esters, |
| | Mangifera Indica (mango) seed butter, Dimethiconol, Argania spinosa |
| | kernel oil (Argan oil), Ethylhexyl Olivate, Glyceryl Ricinoleate, C₁₃-1₅ |
| | Alkane, Cocos nucifera (coconut) oil, Avocado oil, Gluconamido |
| | Amodimethicone, acrylates copolymer, and Polyquaternium-7 |
| 0.01-2 | One or more exfoliators, such as, but not limited: citric acid, |
| | Lactobacillus/Pumpkin Fruit Ferment Filtrate |
| 1-10 | One or more thickeners, such as, but not limited: cetyl |
| | hydroxyethylcellulose, cetearyl alcohol, behenyl alcohol, cetyl alcohol, |
| | hydropropylcelluloses, acrylamidopropyl trimonium chloride/acrylamide |
| | copolymer, lauryl lactate, hydroxypropyl starch phosphate, acrylates |
| | copolymer, ammonium acryloyldimethyltaurate, sodium |
| | acryloyldimethyltaurate/VP copolymer, sodium acrylate/sodium |
| | acryloyldimethyl taurate copolymer, sodium polyacrylate, polyacrylate |
| | crosspolymer-11, carbomer, acrylates/C10-30 alkyl acrylate crosspolymer, |
| | acrylates copolymer, Polyquaternium-73, hydroxyethyl |
| | acrylate/sodiumacryloyldimethyltaurate copolymer, lithium magnesium |
| | sodium silicate, sodium magnesium fluorosilicate, glycol distearate, |
| | hydrogenated castor oil, hydrogenated castor oil hydroxystearate, |
| | hydrogenated castor oil isostearate, hydrogenated castor oil stearate, |
| | hydrogenated castor PEG-8 esters, and Copernicia Cerifera (Carnauba) |
| | wax |
| 0.05-15 | One or more of emulsifiers, such as, but not limited: stearyl alcohol, |
| | crambe abyssinica seed oil phytosterol esters, etearyl alcohol, behenyl |
| | alcohol, Trideceth-12, caprylic/capric/diglyceryl succinate, C14-16 glycol |
| | palmitate, C18-20 glycol isostearate, cocamidopropyl lauryl ether, |
| | dipentaerythrityl hydroxystearate, dipentaerythrityl hydroxyisostearate, |
| | stearoyl glutamic acid, Polysorbate-20, polyglyceryl-10 laurate, |
| | polyglyceryl-6 caprylate, polyglyceryl-3 cocoate, polyglyceryl-4 caprate, |
| | polyglyceryl-6 ricinoleate, polyglyceryl-6 caprylate, sodium surfactin, |
| | C14-22 alcohols, palmitic acid, stearic acid. |
| 0.05-12 | One or more of alcohols, such as, but not limited: cetearyl alcohol, stearyl |
| | alcohol, behenyl alcohol, benzyl alcohol, cetyl alcohol, isopropanol, |
| | propylene glycol, lauric acid, palmitic acid, stearic acid, arachidic alcohol, |
| | isocetyl alcohol, isostearyl alcohol, lauryl alcohol, myristyl alcohol, C30- |
| | 50 alcohols, and lanolin alcohol |
| 0.05-5 | One or more of preservatives, such as, but not limited: benzyl alcohol, and |
| | ethylhexylglycerin, benzoic acid, Xylityl Sesquicaprylate, sodium |
| | benzoate, and potassium sorbate |
| 0.05-8 | One or more of extracts, such as, but not limited: Simmondsia Chinensis |
| | (Jojoba) seed oil, Crambe abyssinica (Abyssinian) Seed oil, Ricinus |
| | communis (castor) seed oil, Helianthus Annuus (sunflower) seed oil, |
| | Raphanus Sativus (Radish) seed extract, Plukenetia Volubilis seed oil, |
| | Salvia Hispanica seed oil, Astrocaryum Vulgare Kernel oil, |
| | Tetrahexyldecyl Ascorbate, Rosmarinus officinalis (rosemary) leaf extract, |
| | Caesalpina Spinosa Fruit extract, Copernicia Cerifera (Carnauba) Wax, or |
| | Helianthus Annuus (sunflower) sprout extract |
| 0.05-5 | One or more of vitamins, such as, but not limited: tocopherol, retinol, |
| | retinyl palmitate, ascorbic acid, tocopherol acetate, and panthenol |
| 0.05-5 | One or more of antioxidants, such as, but not limited: |
| | hydroxyacetophenone, Rosmarinus officinalis (rosemary) leaf extract |
| | (WS), carnosine, tetrahexyl decyl ascorbate, bioflavonoids, lycopene, |
| | daucus carota sativa root cell culture lysate, monoammonium |
| | glycyrrhizinate, alpha lipoic acid (thioctic acid), Leontopodium alpinum |
| | (edelweiss) extract, Vitis vinifera (grape) seed extract, Camellia sinensis |
| | leaf extract, Quercus robur wood extract, hydrolyzed olive fruit, Olea |
| | europea (olive) leaf extract, oleuropin, hydroxyacetophenone, Punica |
| | granatum bark/fruit extract, punicalagin, ellagic acid, polyphenols, |
| | epigallocatechin (EGCG), tannins, sulforaphane, resveratrol, |
| | nordihydroguaiaretic acid |
| 0.5-3 | First component, such as FiberHance ™ bm |
| 0.05-2 | Second component, such as Restart Pro |
| 0.01-5 | pH - Adjuster |

Exemplary Formulation 11 (e.g., leave-in treatment for straight and/or wavy hairs)

| Percent Formula | |
|---|---|
| (% w/w) | Ingredient |
| Adjust to 100 | Water |
| 0.01-1 | One or more chelating agents, such as, but not limited to: Disodium |
| | EDTA, and tetrasodium glutamate diacetate |
| 1-10 | One or more humectants, such as, but not limited to: glycerin, |
| | hydroxyacetophenone, Plukenetia Volubilis seed oil, Salvia Hispanica seed |
| | oil, Undecane, Tridecane, Simmondsia Chinensis (Jojoba) seed oil, |
| | Crambe abyssinica (Abyssinian) Seed oil, Ricinus communis (castor) seed |
| | oil, Helianthus Annuus (sunflower) seed oil, 1,2,6 hexanetriol, |
| | dipropylene glycol, butylene glycol, caprylyl glycol, pentylene glycol, |
| | isopentyldiol, hexylene glycol, phytantriol, propylene glycol, sodium PCA, |
| | sorbitol, triethylene glycol, polyglyceryl sorbitol, glucose, fructose, |
| | panthenol, propanediol, mannitol, xylitol, inositol, trehalose, polydextrose, |
| | potassium PCA, hydrogenated honey, hyaluronic acid, inositol, erythritol, |
| | capryl glycol, and acetamidoethoxyethanol. |
| 0.1-15 | One or more of cationic surfactants, such as, but not limited to: |
| | cetyltrimethyl ammonium chloride (CTAC), polyquaternium-10, |
| | Disunfloweroylethyl Dimonium Chloride, polyquaternium-73, |
| | behenamidopropyl dimethylamine, Stearamidopropyl Dimethylamine, |
| | Polyquaternium-16, Brassicamidopropyl Dimethylamine, polyquaternium- |
| | 7, polyquaternium-37, Hydroxyethyl Cetearamidopropyldimonium |
| | Chloride, Behentrimonium chloride, Cetrimonium Chloride, ethyl lauroyl |
| | arginate HCl, Guar hydroxypropyltrimonium chloride, and |
| | Polyquaternium-11 |
| 0.05-12 | One or more of non-ionic surfactants, such as, but not limited to: C12-20 |
| | alkyl glucoside, trideceth-7, Trideceth-8, crambe abyssinica seed oil |
| | phytosterol esters, lauryl lactyl lactate, Trideceth-12, lauryl glucoside, |
| | Sunfloweroyl methylglucamide, decyl glucoside, coco-glucoside, cocoyl |
| | methyl glucamide, capryloyl/caproyl methyl glucamide, lauroyl/myristoyl |
| | methyl glucamide, cocamidopropylamine oxide, and decylamine oxide |
| 0.25-20 | One or more of emollients, such as, but not limited to: glyceryl stearate, |
| | Dimethicone, Dimethiconol, Argania spinosa kernel oil (Argan oil), |
| | Ethylhexyl Olivate, Squalane, Glyceryl Ricinoleate, Ricinus communis |
| | (castor) seed oil, Gluconamido Amodimethicone, panthenol, propanediol, |
| | Sunflower Seed Oil Glycerides, lauryl lactyl lactate, Ethylhexyl palmitate, |
| | Olea Europaea (olive) fruit unsaponifiables, Caprylic/Capric Triglyceride, |
| | Ethylhexyl Stearate, Crambe Abyssinica seed oil phytosterol esters, |
| | Mangifera Indica (mango) seed butter, Castor oil propanediol esters, |
| | Helianthus annuus (sunflower) seed oil unsaponifiables, Plukenetia |
| | Volubilis seed oil, Salvia Hispanica seed oil, Astrocaryum Vulgare Kernel |
| | oil, Undecane, Tridecane, Simmondsia Chinensis (Jojoba) seed oil, |
| | Crambe abyssinica (Abyssinian) Seed oil, Helianthus Annuus (sunflower) |
| | seed oil, Astrocaryum Murumuru Seed butter, Amodimethicone, |
| | Polycitronellol Acetate, cetyl esters Simmondsia Chinensis (jojoba) butter, |
| | Glycol Distearate, Jojoba Wax PEG-120 esters, Mangifera Indica (mango) |
| | seed butter, C₁₃-1₅ Alkane, Cocos nucifera (coconut) oil, Avocado oil, |
| | acrylates copolymer, and Polyquaternium-7 |
| 0.05-10 | One or more thickeners, such as, but not limited: cetyl |
| | hydroxyethylcellulose, hydropropylcelluloses, cetyl alcohol, behenyl |
| | alcohol, stearic acid, cetearyl alcohol, hydropropylcelluloses, |
| | acrylamidopropyl trimonium chloride/acrylamide copolymer, lauryl |
| | lactate, hydroxypropyl starch phosphate, acrylates copolymer, ammonium |
| | acryloyldimethyltaurate, sodium acryloyldimethyltaurate/VP copolymer, |
| | sodium acrylate/sodium acryloyldimethyl taurate copolymer, sodium |
| | polyacrylate, polyacrylate crosspolymer-11, carbomer, acrylates/C10-30 |
| | alkyl acrylate crosspolymer, acrylates copolymer, Polyquaternium-73, |
| | hydroxyethyl acrylate/sodiumacryloyldimethyltaurate copolymer, lithium |
| | magnesium sodium silicate, sodium magnesium fluorosilicate, glycol |
| | distearate, hydrogenated castor oil, hydrogenated castor oil |
| | hydroxystearate, hydrogenated castor oil isostearate, hydrogenated castor |
| | oil stearate, hydrogenated castor PEG-8 esters, and Copernicia Cerifera |
| | (Carnauba) wax |
| 0.05-10 | One or more of emulsifiers, such as, but not limited: C14-22 alcohols, |
| | glyceryl stearate, cetyl alcohol, stearyl alcohol, behenyl alcohol, palmitic |
| | acid, stearic acid, crambe abyssinica seed oil phytosterol esters, cetearyl |
| | alcohol, behenyl alcohol, Trideceth-12, caprylic/capric/diglyceryl |
| | succinate, C14-16 glycol palmitate, C18-20 glycol isostearate, |
| | cocamidopropyl lauryl ether, dipentaerythrityl hydroxystearate, |
| | dipentaerythrityl hydroxyisostearate, stearoyl glutamic acid, Polysorbate- |
| | 20, polyglyceryl-10 laurate, polyglyceryl-6 caprylate, polyglyceryl-3 |
| | cocoate, polyglyceryl-4 caprate, polyglyceryl-6 ricinoleate, polyglyceryl-6 |
| | caprylate, sodium surfactin, palmitic acid, stearic acid. |
| 0.05-8 | One or more of alcohols, such as, but not limited: cetyl alcohol, stearyl |
| | alcohol, behenyl alcohol, palmitic acid, stearic acid, benzyl alcohol, |
| | propylene glycol, panthenol, propanediol, cetearyl alcohol, isopropanol, |
| | lauric acid, palmitic acid, stearic acid, arachidic alcohol, isocetyl alcohol, |
| | isostearyl alcohol, lauryl alcohol, myristyl alcohol, C30-50 alcohols, and |
| | lanolin alcohol |
| 0.05-5 | One or more of preservatives, such as, but not limited: sodium benzoate, |
| | phenoxyethanol, benzyl alcohol, and ethylhexylglycerin, benzoic acid, |
| | Xylityl Sesquicaprylate, and potassium sorbate |
| 0.05-8 | One or more of extracts, such as, but not limited: Caesalpina Spinosa Fruit |
| | extract, Helianthus Annuus (sunflower) sprout extract, Plukenetia |
| | Volubilis seed oil, Salvia Hispanica seed oil, Astrocaryum Vulgare Kernel |
| | oil, Tetrahexyldecyl Ascorbate, Simmondsia Chinensis (Jojoba) seed oil, |
| | Crambe abyssinica (Abyssinian) Seed oil, Ricinus communis (castor) seed |
| | oil, Helianthus Annuus (sunflower) seed oil, Raphanus Sativus (Radish) |
| | seed extract, Rosmarinus officinalis (rosemary) leaf extract, or Copernicia |
| | Cerifera (Carnauba) Wax |
| 0.05-5 | One or more of vitamins, such as, but not limited: tocopherol, retinol, |
| | retinyl palmitate, ascorbic acid, tocopherol acetate, and panthenol |
| 0.05-5 | One or more of amino acids, such as, but not limited: arginine, histidine, |
| | lysine, aspartic acid, glutamic acid, serine, threonine, asparagine, |
| | glutamine, cysteine, selenocysteine, glycine, proline, alanine, valine, |
| | isoleucine, leucine, methionine, phenylalanine, tyrosine, and tryptophan |
| 0.5-3 | First component, such as FiberHance ™ bm |
| 0.05-2 | Second component, such as Restart Pro |
| 0.01-5 | pH - Adjuster |

Exemplary Formulation 12 (e.g., leave-in treatment for curly and/or coily hairs)

| Percent Formula | |
|---|---|
| (% w/w) | Ingredient |
| Adjust to 100 | Water |
| 0.01-1 | One or more chelating agents, such as, but not limited to: Disodium |
| | EDTA, and tetrasodium glutamate diacetate |
| 1-10 | One or more humectants, such as, but not limited to: glycerin, |
| | hydroxyacetophenone, Plukenetia Volubilis seed oil, Salvia Hispanica seed |
| | oil, Undecane, Tridecane, Simmondsia Chinensis (Jojoba) seed oil, |
| | Crambe abyssinica (Abyssinian) Seed oil, Ricinus communis (castor) seed |
| | oil, Helianthus Annuus (sunflower) seed oil, 1,2,6 hexanetriol, panthenol, |
| | propanediol, dipropylene glycol, butylene glycol, caprylyl glycol, |
| | pentylene glycol, isopentyldiol, hexylene glycol, phytantriol, propylene |
| | glycol, sodium PCA, sorbitol, triethylene glycol, polyglyceryl sorbitol, |
| | glucose, fructose, mannitol, xylitol, inositol, trehalose, polydextrose, |
| | potassium PCA, hydrogenated honey, hyaluronic acid, inositol, erythritol, |
| | capryl glycol, and acetamidoethoxyethanol. |
| 0.1-15 | One or more of cationic surfactants, such as, but not limited to: |
| | polyquaternium-37, Hydroxyethyl Cetearamidopropyldimonium Chloride, |
| | Behentrimonium chloride, Cetrimonium Chloride, ethyl lauroyl arginate |
| | HCl, cetyltrimethyl ammonium chloride (CTAC), polyquaternium-10, |
| | Disunfloweroylethyl Dimonium Chloride, polyquaternium-73, |
| | behenamidopropyl dimethylamine, Stearamidopropyl Dimethylamine, |
| | Polyquaternium-16, Brassicamidopropyl Dimethylamine, polyquaternium- |
| | 7, Guar hydroxypropyltrimonium chloride, and Polyquaternium-11 |
| 0.25-20 | One or more of emollients, such as, but not limited to: panthenol, |
| | propanediol Copernicia Cerifera (Carnauba) Wax, C₁₃-₁₅ Alkane, Glycol |
| | Distearate, Cocos nucifera (coconut) oil, Avocado oil, Glyceryl |
| | Ricinoleate, Ricinus communis (castor) seed oil, Dimethicone, |
| | Dimethiconol, Argania spinosa kernel oil (Argan oil), Ethylhexyl Olivate, |
| | Squalane, Gluconamido Amodimethicone, , glyceryl stearate, Sunflower |
| | Seed Oil Glycerides, lauryl lactyl lactate, Ethylhexyl palmitate, Olea |
| | Europaea (olive) fruit unsaponifiables, Caprylic/Capric Triglyceride, |
| | Ethylhexyl Stearate, Crambe Abyssinica seed oil phytosterol esters, |
| | Mangifera Indica (mango) seed butter, Castor oil propanediol esters, |
| | Helianthus annuus (sunflower) seed oil unsaponifiables, Plukenetia |
| | Volubilis seed oil, Salvia Hispanica seed oil, Astrocaryum Vulgare Kernel |
| | oil, Undecane, Tridecane, Simmondsia Chinensis (Jojoba) seed oil, |
| | Crambe abyssinica (Abyssinian) Seed oil, Helianthus Annuus (sunflower) |
| | seed oil, Astrocaryum Murumuru Seed butter, Amodimethicone, |
| | Polycitronellol Acetate, cetyl esters Simmondsia Chinensis (jojoba) butter, |
| | Jojoba Wax PEG-120 esters, acrylates copolymer, and Polyquaternium-7 |
| 0.05-10 | One or more thickeners, such as, but not limited: stearic acid, cetyl alcohol, |
| | acrylamidopropyl trimonium chloride/acrylamide copolymer, lauryl |
| | lactate, hydroxypropyl starch phosphate, acrylates copolymer, ammonium |
| | acryloyldimethyltaurate, sodium acryloyldimethyltaurate/VP copolymer, |
| | sodium acrylate/sodium acryloyldimethyl taurate copolymer, sodium |
| | polyacrylate, polyacrylate crosspolymer-11, carbomer, acrylates/C10-30 |
| | alkyl acrylate crosspolymer, acrylates copolymer, cetyl |
| | hydroxyethylcellulose, hydropropylcelluloses, cetearyl alcohol, Copernicia |
| | Cerifera (Carnauba) wax, behenyl alcohol, Polyquaternium-73, |
| | hydroxyethyl acrylate/sodiumacryloyldimethyltaurate copolymer, lithium |
| | magnesium sodium silicate, sodium magnesium fluorosilicate, glycol |
| | distearate, hydrogenated castor oil, hydrogenated castor oil |
| | hydroxystearate, hydrogenated castor oil isostearate, hydrogenated castor |
| | oil stearate, and hydrogenated castor PEG-8 esters |
| 0.05-10 | One or more of emulsifiers, such as, but not limited: behenyl alcohol, |
| | cetearyl alcohol, palmitic acid, stearic acid, C14-22 alcohols, glyceryl |
| | stearate, cetyl alcohol, stearyl alcohol, crambe abyssinica seed oil |
| | phytosterol esters, behenyl alcohol, Trideceth-12, |
| | caprylic/capric/diglyceryl succinate, C14-16 glycol palmitate, C18-20 |
| | glycol isostearate, cocamidopropyl lauryl ether, dipentaerythrityl |
| | hydroxystearate, dipentaerythrityl hydroxyisostearate, stearoyl glutamic |
| | acid, Polysorbate-20, polyglyceryl-10 laurate, polyglyceryl-6 caprylate, |
| | polyglyceryl-3 cocoate, polyglyceryl-4 caprate, polyglyceryl-6 ricinoleate, |
| | polyglyceryl-6 caprylate, sodium surfactin, palmitic acid, stearic acid. |
| 0.05-8 | One or more of alcohols, such as, but not limited: cetyl alcohol, stearyl |
| | alcohol, behenyl alcohol, palmitic acid, stearic acid, benzyl alcohol, |
| | propylene glycol, panthenol, propanediol, cetearyl alcohol, isopropanol, |
| | lauric acid, palmitic acid, stearic acid, arachidic alcohol, isocetyl alcohol, |
| | isostearyl alcohol, lauryl alcohol, myristyl alcohol, C30-50 alcohols, and |
| | lanolin alcohol |
| 0.05-5 | One or more of preservatives, such as, but not limited: sodium benzoate, |
| | phenoxyethanol, benzyl alcohol, and ethylhexylglycerin, benzoic acid, |
| | Xylityl Sesquicaprylate, and potassium sorbate |
| 0.05-8 | One or more of extracts, such as, but not limited: Caesalpina Spinosa Fruit |
| | extract, Helianthus Annuus (sunflower) sprout extract, Plukenetia |
| | Volubilis seed oil, Salvia Hispanica seed oil, Astrocaryum Vulgare Kernel |
| | oil, Tetrahexyldecyl Ascorbate, Simmondsia Chinensis (Jojoba) seed oil, |
| | Crambe abyssinica (Abyssinian) Seed oil, Ricinus communis (castor) seed |
| | oil, Helianthus Annuus (sunflower) seed oil, Raphanus Sativus (Radish) |
| | seed extract, Rosmarinus officinalis (rosemary) leaf extract, or Copernicia |
| | Cerifera (Carnauba) Wax |
| 0.05-5 | One or more of vitamins, such as, but not limited: tocopherol, retinol, |
| | retinyl palmitate, ascorbic acid, tocopherol acetate, and panthenol |
| 0.05-5 | One or more of amino acids, such as, but not limited: arginine, histidine, |
| | lysine, aspartic acid, glutamic acid, serine, threonine, asparagine, |
| | glutamine, cysteine, selenocysteine, glycine, proline, alanine, valine, |
| | isoleucine, leucine, methionine, phenylalanine, tyrosine, and tryptophan |
| 0.5-3 | First component, such as FiberHance ™ bm |
| 0.05-2 | Second component, such as Restart Pro |
| 0.01-5 | pH - Adjuster |

In each of the exemplary formulations:
The first component contains hydroxypropylgluconamide and hydroxypropylammonium gluconate; preferably 50% hydroxypropylgluconamide and hydroxypropylammonium gluconate, 49% water, and 1% preservatives (such as benzyl alcohol, sodium benzoate, potassium sorbate) and pH adjuster (such as tartaric acid); and more preferably, the first component is FiberHance™ bm; and
The second component contains dicarboxylic acid, amino acid, provitamin B5 and polysaccharides extracted from seeds of Flaxseed (Linum usitatissimum) and Chia (Salvia hispanica), preferably 30-70% water, 5-20% itaconic acid, 5-20% arginine, 1-15% panthenol, 1-10% Linum usitatissimum (Linseed) seed extract, 1-9% Salvia hispanica seed extract, 1-8% polyglyceryl-10 Laurate, 0.2-0.9% xylitol, and 0.2-0.9% caprylic acid; and more preferably the second component is Restart Pro.

### Example 3: Sensory Effects of all Hair Types after Hair Treatment

Hair compositions according to the application were prepared. Sensory effects of the hair compositions on damaged all hair types were evaluated.

In particular, the following three hair products were prepared: shampoo and conditioner bundle, shampoo and mask bundle, and shampoo, conditioner and leave-in treatment (LIT) bundle. These hair products were tested on subjects with damaged hair types, including straight, wavy, curly, and coily hairs. The subjects were provided with the following instructions for the application of the hair products:
Shampoo directions: wet hair completely, apply a generous amount and massage through hair and scalp for 1 minute, then rinse.
Conditioner Application: section wet hair and apply conditioner from root to tips, leave on for 1-3 minute, then rinse.
Mask Application: section wet hair and apply conditioner from root to tips, leave on for 3-5 minute, then rinse.
LIT Application: post shower (damp [towel dry wet])—Start with 1 pump for finer and short hair and increase the usage amount with increasing hair density and length. Spread the product evenly throughout the hair with fingers/palms of your hands on the side that is getting the treatment. Air dry or blow dry/style as usual.

The subjects were instructed to record the wet and dry feel and complete questionnaires evaluating the sensory effects (e.g., wet feel and dry feel) of hair products tested.

Wet feel was evaluated based on 5 parameters, such as: Flash Foam, Foam Density, Wet Conditioned Feel, Web comb/Detangling, and Rinsability (1=hard to rinse, 5=easy to rinse).

Dry feel was evaluated based on 12 parameters, such as: Hair is soft; Hair has shine; Dry Comb/dry detangling; Hair ends are smooth; “Hair feels greasy (1=very greasy, 5=clean fee)”; “No visible residue (1=residue/dull, 5=no residue)”; Conditioned hair feel (1=coated, 5=clean conditioned feel); Hair feels repaired; Hair is manageable; Curl Definition; Hair does not feel weighed down; Hair has no frizz.

Different points (from 1 to 5) were given based on the sensory effects on damaged all hair types, with 5 being the highest score, and 1 being the lowest score.

Results on three different experiments are summarized in Tables F, G, and H below.

TABLE C
Sensory effects of shampoo and conditioner bundle test on damaged all hair types

| | Shampoo and Conditioner bundle test | | | | | | | | | | | | | Average |
|---|---|---|---|---|---|---|---|---|---|---|---|---|---|---|
| | on damaged all hair types | | | | | | | | | | | | | scores: |
| Wet | Flash Foam | 5 | 5 | 5 | 5 | 5 | 5 | 3 | 5 | 4 | 5 | 4 | 5 | 4.67 |
| feel | Foam | 5 | 5 | 4 | 4 | 4 | 4 | 3 | 5 | 4 | 5 | 4 | 5 | 4.33 |
| | Density | | | | | | | | | | | | | |
| | Wet | 4 | 4 | 5 | 5 | 4 | 5 | 3 | 5 | 5 | 5 | 3 | 5 | 4.42 |
| | Conditioned | | | | | | | | | | | | | |
| | Feel | | | | | | | | | | | | | |
| | Web comb/ | 5 | 3 | 5 | 5 | 4 | 4 | 2 | 5 | N/a | 5 | 3 | 5 | 4.18 |
| | Detangling | | | | | | | | | | | | | |
| | Rinsability | 5 | 5 | 5 | 5 | 5 | 5 | 4 | 4 | 5 | 5 | 5 | 5 | 4.83 |
| | (1 = hard to | | | | | | | | | | | | | |
| | rinse, 5 = easy | | | | | | | | | | | | | |
| | to rinse) | | | | | | | | | | | | | |
| Dry | Hair is soft | 5 | 4 | 3 | 5 | 5 | 5 | 5 | 4 | 5 | 5 | 3 | 5 | 4.50 |
| feel | Hair has | 5 | 5 | 5 | 5 | 5 | 5 | 3 | 3 | 5 | 5 | 3 | N/A | 4.45 |
| | shine | | | | | | | | | | | | | |
| | Dry | 5 | 4 | 4 | 4 | 5 | 4 | 3 | 3 | N/A | 5 | 4 | N/A | 4.10 |
| | Comb/dry | | | | | | | | | | | | | |
| | detangling | | | | | | | | | | | | | |
| | Hair ends are | 5 | 5 | 3 | 4 | 4 | 4 | 4 | 3 | 4 | 5 | 3 | 5 | 4.08 |
| | smooth | | | | | | | | | | | | | |
| | Hair feels | 5 | 5 | 5 | 5 | 5 | 5 | 4 | 5 | 5 | 5 | 5 | 5 | 4.92 |
| | greasy | | | | | | | | | | | | | |
| | (1 = very | | | | | | | | | | | | | |
| | greasy, 5 = | | | | | | | | | | | | | |
| | clean fee) | | | | | | | | | | | | | |
| | No visible | 5 | 5 | 5 | 5 | 5 | 5 | 5 | 5 | 5 | 5 | 5 | 5 | 5.00 |
| | residue | | | | | | | | | | | | | |
| | (1 = | | | | | | | | | | | | | |
| | residue/dull, | | | | | | | | | | | | | |
| | 5 = no | | | | | | | | | | | | | |
| | residue) | | | | | | | | | | | | | |
| | Conditioned | 5 | 5 | 4 | 5 | 5 | 5 | 4 | 5 | 5 | 5 | 3 | 5 | 4.67 |
| | hair feel (1 = | | | | | | | | | | | | | |
| | coated, 5 = | | | | | | | | | | | | | |
| | clean | | | | | | | | | | | | | |
| | conditioned | | | | | | | | | | | | | |
| | feel) | | | | | | | | | | | | | |
| | Hair feels | 4 | 5 | 4 | 4 | 5 | 5 | 3 | 3 | 3 | 5 | 3 | N/A | 4.00 |
| | repaired | | | | | | | | | | | | | |
| | Hair is | 5 | 5 | 5 | 5 | 5 | 5 | 5 | 3 | 5 | 5 | 3 | 5 | 4.67 |
| | manageable | | | | | | | | | | | | | |
| | Curl | NA | NA | NA | NA | NA | NA | 4 | 3 | 5 | 5 | N/A | N/A | 4.25 |
| | Definition | | | | | | | | | | | | | |
| | Hair does | 5 | 5 | 5 | 5 | 5 | 5 | 4 | 3 | 5 | 5 | 5 | 5 | 4.75 |
| | not feel | | | | | | | | | | | | | |
| | weighed | | | | | | | | | | | | | |
| | down | | | | | | | | | | | | | |
| | Hair has no | 5 | 5 | 4 | 4 | 4 | 5 | 5 | 2 | 3 | 5 | 4 | N/A | 4.18 |
| | frizz | | | | | | | | | | | | | |

TABLE D
Sensory effects of shampoo and mask bundle test on damaged all hair types

| | Shampoo and Mask bundle test | | | | | | | | | | | | | | Average |
|---|---|---|---|---|---|---|---|---|---|---|---|---|---|---|---|
| | on damaged all hair types | | | | | | | | | | | | | | scores: |
| Dry | Hair is soft | 5 | 5 | 5 | 5 | 5 | 5 | 5 | 4 | 5 | 5 | 3 | 4 | 5 | 4.69 |
| feel | Hair has | 4 | 5 | 5 | 5 | 4 | 5 | 5 | 4 | 5 | N/A | 4 | 4 | 5 | 4.58 |
| | shine | | | | | | | | | | | | | | |
| | Hair ends | 4.5 | 4 | 5 | 5 | 4 | 5 | 5 | 4 | 5 | 5 | 3 | 3 | 5 | 4.42 |
| | are smooth | | | | | | | | | | | | | | |
| | Conditioned | 4.5 | 4 | 5 | 5 | 5 | 4 | 5 | 4 | 5 | 5 | 5 | 5 | 5 | 4.73 |
| | hair feel | | | | | | | | | | | | | | |
| | (1 = coated, | | | | | | | | | | | | | | |
| | 5 = clean | | | | | | | | | | | | | | |
| | conditioned | | | | | | | | | | | | | | |
| | feel) | | | | | | | | | | | | | | |
| | No visible | 5 | 4 | 4 | 4 | 5 | 5 | 5 | 5 | 5 | 5 | 5 | 5 | 5 | 4.77 |
| | residue | | | | | | | | | | | | | | |
| | (1 = residue/ | | | | | | | | | | | | | | |
| | dull, 5 = no | | | | | | | | | | | | | | |
| | residue) | | | | | | | | | | | | | | |
| | Hair is | 5 | 5 | NA | 5 | 5 | 4 | 5 | 5 | 5 | 5 | 4 | 3 | 4 | 4.58 |
| | manageable | | | | | | | | | | | | | | |
| | Hair looks | 5 | 5 | 3 | 4 | 4 | 4 | 5 | 5 | 4 | 5 | 5 | 3 | 4 | 4.31 |
| | repaired/ | | | | | | | | | | | | | | |
| | healthy | | | | | | | | | | | | | | |
| | Touchable | 5 | 5 | 5 | 4 | 5 | 5 | 5 | 5 | 4 | 5 | 5 | 5 | 5 | 4.85 |
| | hair | | | | | | | | | | | | | | |
| | Hair feels | 5 | 4 | 4 | 5 | 5 | 5 | 5 | 5 | 4 | 5 | 4 | 5 | 5 | 4.69 |
| | greasy | | | | | | | | | | | | | | |
| | (1 = very | | | | | | | | | | | | | | |
| | greasy, 5 = | | | | | | | | | | | | | | |
| | clean feel) | | | | | | | | | | | | | | |
| | Curl | NA | NA | NA | NA | NA | 4 | 5 | 4 | 3 | 5 | 4 | 5 | 5 | 4.38 |
| | Definition | | | | | | | | | | | | | | |
| | Hair has no | 4.5 | 4 | 4 | 4 | 4 | 4 | 5 | 4 | 5 | 5 | 4 | 2 | 5 | 4.19 |
| | frizz | | | | | | | | | | | | | | |

TABLE E
Sensory effects of shampoo, conditioner and leave-in
treatment (LIT) bundle test on damaged all hair types

| | Shampoo, Conditioner, LIT bundle test | | | | | | | | | | | | | | | Average |
|---|---|---|---|---|---|---|---|---|---|---|---|---|---|---|---|---|
| | on damaged all hair types | | | | | | | | | | | | | | | scores: |
| Wet | Flowing | 4.5 | NA | NA | 5 | NA | 4 | 5 | 5 | 5 | 5 | 5 | 4 | 5 | 4 | 4.75 |
| feel | Characteristics | | | | | | | | | | | | | | | |
| | Wet | 4.5 | NA | NA | 4 | NA | 4 | 5 | 5 | 5 | 5 | 5 | 5 | 5 | 5 | 4.25 |
| | conditioned/ | | | | | | | | | | | | | | | |
| | cushioned feel | | | | | | | | | | | | | | | |
| | Web comb/ | 5 | NA | 5 | 4 | NA | 4 | 4 | 5 | 5 | 5 | 5 | 5 | 4 | 5 | 4.67 |
| | Detangling | | | | | | | | | | | | | | | |
| Dry | Hair is soft | 4.5 | 4 | 5 | 5 | 5 | 5 | 5 | 4 | 5 | 3 | 5 | 5 | 5 | 5 | 4.70 |
| feel | Hair has shine | 4 | 4 | 4 | 5 | 4 | 3 | 5 | 4 | 4 | 3 | 4 | 5 | 5 | 4 | 4.20 |
| | Hair ends are | 4 | 4 | 5 | 5 | 5 | 4 | 5 | 5 | 5 | 3 | 4 | 5 | 5 | 4 | 4.60 |
| | smooth | | | | | | | | | | | | | | | |
| | Conditioned | 4 | 3 | 5 | 4 | 5 | 5 | 5 | 4 | 5 | 5 | 5 | 5 | 5 | 5 | 4.20 |
| | hair feel (1 = | | | | | | | | | | | | | | | |
| | coated, 5 = | | | | | | | | | | | | | | | |
| | clean | | | | | | | | | | | | | | | |
| | conditioned | | | | | | | | | | | | | | | |
| | feel) | | | | | | | | | | | | | | | |
| | No visible | 5 | 4 | 5 | 5 | 5 | 5 | 5 | 4 | 5 | 5 | 5 | 5 | 5 | 5 | 4.80 |
| | residue | | | | | | | | | | | | | | | |
| | (1 = | | | | | | | | | | | | | | | |
| | residue/dull, | | | | | | | | | | | | | | | |
| | 5 = no residue) | | | | | | | | | | | | | | | |
| | Hair is bouncy | 4 | 4 | 5 | 5 | 3 | 5 | 5 | 4 | 5 | 4 | 5 | 4 | 5 | 5 | 4.20 |
| | and manageable | | | | | | | | | | | | | | | |
| | Hair looks | 4.5 | 3 | 5 | 5 | 5 | 3 | 5 | 4 | 4 | 3 | 4 | 4 | 5 | 5 | 4.50 |
| | repaired/healthy | | | | | | | | | | | | | | | |
| | Hair has no | 4.5 | 4 | 4 | 5 | 4 | 3 | 4 | 4 | 5 | 3 | 4 | 4 | 5 | 4.5 | 4.30 |
| | frizz | | | | | | | | | | | | | | | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the applications as defined by the specific description.

We claim:

1. A hair composition, comprising:
(1) a first component comprising at least one of a compound of Formula (I) and a compound of Formula (II) 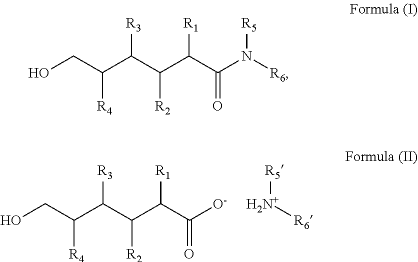
wherein R₁-R₄ and R₁′-R₄′ are independently hydrogen, a hydrocarbon radical having 1 to 10 carbon atoms, a hydroxyl group, an amino group, a sulfhydryl group, an aryl group, or a halogen, and
R₅, R₆, R₅′ and R₆′ are independently hydrogen, an aliphatic hydrocarbon group, an alicyclic hydrocarbon group, an aryl group, an alkylaryl group or a heterocyclic group, with the proviso that R₅′ and R₆′ are not simultaneously hydrogen, wherein the aliphatic hydrocarbon group, the alicyclic hydrocarbon group, the aryl group, the alkylaryl group or the heterocyclic group is substituted with at least one hydroxyl group; and
(2) a second component comprising:
a. an α,β unsaturated dicarboxylic acid and/or an ester thereof:
b. a nonionic crosslinking agent selected from the group consisting of polyols, saccharides or a combination thereof; and
c. an ionic cross-linking agent selected from the group consisting of hydrolyzed proteins, amino acids or a combination thereof.

2. The hair composition of claim 1, wherein:
(1) the first component comprises the compound of Formula (I) and the compound of Formula (II); R₁-R₄ and R₁′-R₄′ are each a hydroxyl group, and R₅, R₆, R₅′ and R₆′ are independently hydrogen, or a —CH₂CH₂CH₂OH group;
(2) the α,β unsaturated dicarboxylic acid in the second component comprises itaconic acid, acrylic acid, maleic acid, fumaric acid, or methacrylic acid;
(3) the nonionic crosslinking agent in the second component comprises panthenol or propanediol;
(4) the ionic cross-linking agent in the second component comprises an amino acid, wherein the amino acid is selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan;
(5) the second component further comprises one or more plant or seed extracts;
(6) the second component further comprises an ester;
(7) the total amount of the first component is about 0.2% to 2.5% by weight based on the total weight of the hair composition; and/or
(8) the total amount of the second component is about 0.05% to 2% by weight based on the total weight of the hair composition.

3. The hair composition of claim 2, wherein the second component further comprises one or more plant or seed extracts selected from the group consisting of Linum usitatissimum (Linseed) Seed Extract, Salvia hispanica (Chia) Seed Extract; and an ester of glycerol and a saturated or unsaturated, branched or unbranched carboxylic acid.

4. The hair composition of claim 3, wherein the second component further comprises polyglyceryl-10 Laurate.

5. The hair composition of claim 2, further comprising at least one additional component selected from the group consisting of surfactants, fatty acid soap, hair and skin conditioning agents, suspending aids, emollients, emulsifiers, rheology modifiers, thickening agents, vitamins, hair growth promoters, self-tanning agents, sunscreens, skin lighteners, anti-aging compounds, anti-wrinkle compounds, hair coloring treatment agents, hair color protecting agents, hair styling products, anti-cellulite compounds, anti-acne compounds, anti-dandruff agents, anti-inflammatory compounds, analgesics, antiperspirant agents, deodorant agents, hair fixatives, particulates, abrasives, moisturizers, antioxidants, keratolytic agents, anti-static agents, foam boosters, hydrotropes, solubilizing agents, chelating agents, antimicrobial agents, antifungal agents, pH adjusting agents, buffering agents, botanicals, hair colorants, oxidizing agents, reducing agents, hair and skin bleaching agents, pigments, anticaries, anti-tartar agents, anti-plaque agents, solvents, preservatives, humectants, exfoliators, natural extracts, alcohols, water, and combinations thereof.

6. The hair composition of claim 5, further comprising:
(1) one or more preservatives selected from the group consisting of sodium benzoate, phenoxyethanol, benzyl alcohol, ethylhexylglycerin, benzoic acid, xylityl sesquicaprylate, ethylhexylglycerin, caprylic acid, itaconic acid, benzoic acid, potassium sorbate, and combinations thereof;
(2) one or more surfactants selected from the group consisting of a cationic surfactant, non-ionic surfactant, amphoteric surfactant, and anionic surfactant; wherein:
the one or more cationic surfactants are selected from the group consisting of polyquaternium-37, hydroxyethyl cetearamidopropyldimonium chloride, behentrimonium chloride, cetrimonium chloride, ethyl lauroyl arginate HCl, cetyltrimethyl ammonium chloride (CTAC), polyquaternium-10, disunfloweroylethyl dimonium chloride, polyquaternium-73, behenamidopropyl dimethylamine, stearamidopropyl dimethylamine, polyquaternium-16, brassicamidopropyl dimethylamine, polyquaternium-7, Guar hydroxypropyltrimonium chloride, polyquaternium-11, and combinations thereof;
the one or more cationic surfactants are selected from the group consisting of a polyquaternium, polyquaternium-10, polyquaternium-7 and polyquaternium-73, Guar hydroxypropyltrimonium chloride, behenamidopropyl dimethylamine, polyquaternium-37, behentrimonium chloride, brassicamidopropyl dimethylamine, stearamidopropyl dimethylamine, polyquaternium-16, disunfloweroylethyl dimonium chloride, cetrimonium chloride, hydroxyethyl cetearamidopropyldimonium chloride, and polyquaternium-11;
the one or more non-ionic surfactants are selected from the group consisting of C₁₂₋₂₀ alkyl glucoside, trideceth-7, Trideceth-8, Crambe abyssinica seed oil phytosterol esters, lauryl lactyl lactate, Trideceth-12, lauryl glucoside, Sunfloweroyl methylglucamide, decyl glucoside, coco-glucoside, cocoyl methyl glucamide, capryloyl/caproyl methyl glucamide, lauroyl/myristoyl methyl glucamide, cocamidopropylamine oxide, decylamine oxide, and combinations thereof;
the one or more amphoteric surfactants are selected from the group consisting of betaine, cocamidopropyl hydroxysultaine, lauryl betainec, and combinations thereof; and/or
the one or more anionic surfactants are selected from the group consisting of sodium lauroyl methyl isethionate, sodium cocoyl glutamate, cocamidopropyl hydroxysultaineutamate, disodium cocoyl glutamate, sodium lauroyl lactylate, sodium methyl cocoyl taurate, sodium lauroyl sarcosinate, and combinations thereof;
(3) one or more emollients selected from the group consisting of panthenol, propanediol opernicia Cerifera (Carnauba) Wax, C13-15 alkane, glycol distearate, Cocos nucifera (coconut) oil, avocado oil, glyceryl ricinoleate, Ricinus communis (castor) seed oil, dimethicone, dimethiconol, Argania spinosa kernel oil (Argan oil), ethylhexyl olivate, squalane, gluconamido amodimethicone, glyceryl stearate, Sunflower Seed Oil Glycerides, lauryl lactyl lactate, ethylhexyl palmitate, Olea Europaea (olive) fruit unsaponifiables, caprylic/capric triglyceride, ethylhexyl stearate, Crambe Abyssinica seed oil phytosterol esters, Mangifera Indica (mango) seed butter, castor oil propanediol esters, Helianthus annuus (sunflower) seed oil unsaponifiables, Plukenetia volubilis seed oil, Salvia hispanica seed oil, Astrocaryum vulgare Kernel oil, undecane, tridecane, Simmondsia chinensis (Jojoba) seed oil, Crambe abyssinica (Abyssinian) seed oil, Helianthus annuus (sunflower) seed oil, Astrocaryum murumuru Seed butter, amodimethicone, polycitronellol acetate, cetyl esters Simmondsia chinensis (jojoba) butter, Jojoba Wax PEG-120 esters, acrylates copolymer, Polyquaternium-7, and combinations thereof;
(4) one or more thickening agents, wherein the thickening agents comprise cetyl hydroxyethylcellulose, hydropropylcelluloses, cetearyl alcohol, Copernicia cerifera (Carnauba) wax, behenyl alcohol, Raphanus sativus (Radish) seed extract, Rosmarinus officinales (rosemary) leaf extract, Tetrahexyldecyl Ascorbate, Caesalpina spinosa Fruit extract, Helianthus annuus (sunflower) sprout extract, cetyl alcohol, acrylamidopropyl trimonium chloride/acrylamide copolymer, acrylates copolymer-4, lauryl lactate, hydroxypropyl starch phosphate, acrylates copolymer, ammonium acryloyldimethyltaurate, sodium acryloyldimethyltaurate/VP copolymer, sodium acrylate/sodium acryloyldimethyl taurate copolymer, sodium polyacrylate, polyacrylate crosspolymer-11, carbomer, acrylates/C10-30 alkyl acrylate crosspolymer, acrylates copolymer, Polyquaternium-73, hydroxyethyl acrylate/sodiumacryloyldimethyltaurate copolymer, lithium magnesium sodium silicate, sodium magnesium fluorosilicate, glycol distearate, hydrogenated castor oil, hydrogenated castor oil hydroxystearate, hydrogenated castor oil isostearate, hydrogenated castor oil stearate, hydrogenated castor PEG-8 esters, or combinations thereof;
(5) one or more chelating agents selected from the group consisting of an EDTA or salt thereof, tetrasodium glutamate diacetate, or combinations thereof;
(6) one or more humectants, wherein the humectants comprise panthenol, propanediol, glycerin, hydroxyacetophenone, Plukenetia volubilis seed oil, Salvia hispanica seed oil, Undecane, Tridecane, Simmondsia chinensis (Jojoba) seed oil, Crambe abyssinica (Abyssinian) Seed oil, Ricinus communis (castor) seed oil, Helianthus annuus (sunflower) seed oil, 1,2,6 hexanetriol, dipropylene glycol, butylene glycol, caprylyl glycol, pentylene glycol, isopentyldiol, hexylene glycol, phytantriol, propylene glycol, sodium PCA, sorbitol, triethylene glycol, polyglyceryl sorbitol, glucose, fructose, mannitol, xylitol, inositol, trehalose, polydextrose, potassium PCA, hydrogenated honey, hyaluronic acid, inositol, erythritol, capryl glycol, acetamidoethoxyethanol, or combinations thereof;
(7) one or more exfoliators, wherein the exfoliators comprise citric acid, Lactobacillus/Pumpkin Fruit Ferment Filtrate, or combinations thereof;
(8) one or more natural extracts, wherein the extracts comprise Caesalpina spinosa Fruit extract, Helianthus annuus (sunflower) sprout extract, Plukenetia volubilis seed oil, Salvia hispanica seed oil, Astrocaryum vulgare Kernel oil, Tetrahexyldecyl Ascorbate, Simmondsia chinensis (Jojoba) seed oil, Crambe abyssinica (Abyssinian) Seed oil, Ricinus communis (castor) seed oil, Helianthus annuus (sunflower) seed oil, Linum usitatissimum (Linseed) Seed Extract, Raphanus sativus (Radish) seed extract, Rosmarinus officinalis (rosemary) leaf extract, Copernicia cerifera (Carnauba) Wax, or combinations thereof;
(9) one or more vitamins, wherein the vitamins comprise tocopherol, panthenol, retinol, retinyl palmitate, ascorbic acid, tocopherol acetate, or combinations thereof;
(10) one or more antioxidants, wherein the antioxidants comprise hydroxyacetophenone, Rosmarinus officinalis (rosemary) leaf extract (WS), carnosine, tetrahexyl decyl ascorbate, bioflavonoids, lycopene, or combinations thereof;
(11) one or more emulsifiers, wherein the emulsifiers comprise behenyl alcohol, cetearyl alcohol, palmitic acid, stearic acid, C14-22 alcohols, glyceryl stearate, cetyl alcohol, stearyl alcohol, Crambe abyssinica seed oil phytosterol esters, Trideceth-12, caprylic/capric/diglyceryl succinate, stearic acid, C14-16 glycol palmitate, C18-20 glycol isostearate, cocamidopropyl lauryl ether, dipentaerythrityl hydroxystearate, dipentaerythrityl hydroxyisostearate, stearoyl glutamic acid, Polysorbate-20, polyglyceryl-10 laurate, polyglyceryl-6 caprylate, polyglyceryl-3 cocoate, polyglyceryl-4 caprate, polyglyceryl-6 ricinoleate, polyglyceryl-6 caprylate, sodium surfactin, palmitic acid, or combinations thereof;
(12) one or more alcohols, wherein the alcohols comprise cetyl alcohol, stearyl alcohol, behenyl alcohol, palmitic acid, stearic acid, benzyl alcohol, propylene glycol, panthenol, propanediol, cetearyl alcohol, isopropanol, stearic acid, lauric acid, palmitic acid, arachidic alcohol, isocetyl alcohol, isostearyl alcohol, lauryl alcohol, myristyl alcohol, C30-50 alcohols, lanolin alcohol, or combinations thereof; and/or
(13) one or more amino acids, wherein the amino acids comprise arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, or combinations thereof.

7. The hair composition of claim 6, further comprising:
(1) the one or more preservatives selected from the group consisting of a benzoic acid or salt thereof, xylityl sesquicaprylate, benzyl alcohol, or ethylhexylglycerin;
(2) the one or more non-ionic surfactants selected from the group consisting of lauryl glucoside, Sunfloweroyl methylglucamide;
(3) the one or more amphoteric surfactants selected from the group consisting of cocamidopropyl hydroxysultaine, or lauryl betaine;
(4) the one or more anionic surfactants selected from the group consisting of sodium lauroyl methyl isethionate, sodium cocoyl glutamate, disodium cocoyl glutamate, sodium lauroyl lactylate, sodium methyl cocoyl taurate, or sodium lauroyl sarcosinate;
(5) the one or more emollients selected from the group consisting of Glycol Distearate, Jojoba Wax PEG-120 esters, Dimethicone, Ethylhexyl palmitate, Olea Europaea (olive) fruit unsaponifiables, Caprylic/Capric Triglyceride, Ethylhexyl Stearate, Castor oil propanediol esters, Crambe Abyssinica seed oil phytosterol, Helianthus annuus (sunflower) seed oil, Simmondsia chinensis (jojoba) butter, Astrocaryum murumuru Seed butter, Plukenetia volubilis seed oil, Salvia hispanica seed oil, Polycitronellol Acetate, Undecane, Tridecane, Simmondsia chinensis (Jojoba) seed oil, Crambe abyssinica (Abyssinian) Seed oil, Ricinus communis (castor) seed oil, Helianthus annuus (sunflower) seed oil unsaponifiables, Mangifera Indica (mango) seed butter, Squalane, Astrocaryum vulgare Kernel oil, Dimethiconol, Argania spinosa kernel oil (Argan oil), Ethylhexyl Olivate, Glyceryl Ricinoleate, C₁₃₋₁₅ Alkane, Cocos nucifera (coconut) oil, Avocado oil, or Gluconamido Amodimethicone; and/or
(6) the one or more thickening agent comprising acrylates copolymer, or Polyquaternium-73.

8. The hair composition of claim 2, further comprising a chelating agent; a moisturizer; an exfoliator; and/or an acid.

9. The hair composition of claim 8, further comprising EDTA, glycerin, Lactobacillus/Pumpkin Fruit Ferment Filtrate, and/or citric acid.

10. The hair composition of claim 8, further comprising water; one or more pH adjusting agents; Betaine; and/or natural extracts.

11. The hair composition of claim 10, further comprising water; one or more pH adjusting agents and the composition has a pH value of 4-6; Betaine at the amount of 1% to 15% by weight; and/or natural extracts comprising one or more of Raphanus sativus (Radish) seed extract, Rosmarinus officinales (rosemary) leaf extract, Tetrahexyldecyl Ascorbate, Caesalpina spinosa Fruit extract, or Helianthus annuus (sunflower) sprout extract.

12. A hair composition, comprising:
(1) a first component containing hydroxypropylgluconamide and hydroxypropylammonium gluconate; and
(2) a second component containing dicarboxylic acid, amino acid, provitamin B5 and polysaccharides extracted from seeds of Flaxseed (Linum usitatissimum) and Chia (Salvia hispanica),
wherein, the total amount of the first component is about 0.1% to 5% by weight based on the total weight of the hair composition; and/or the total amount of the second component is about 0.05% to 2% by weight based on the total weight of the hair composition.

13. The hair composition of claim 12, wherein:
(1) the first component contains 50% hydroxypropylgluconamide and hydroxypropylammonium gluconate, 49% water, and 1% preservatives selected from the group consisting of benzyl alcohol, sodium benzoate, and potassium sorbate, and a pH adjuster; and
(2) the second component contains 30-70% water, 5-20% itaconic acid, 5-20% arginine, 1-15% panthenol, 1-10% Linum usitatissimum (Linseed) seed extract, 1-9% Salvia hispanica seed extract, 1-8% polyglyceryl-10 Laurate, 0.2-0.9% xylitol, and 0.2-0.9% caprylic acid,
wherein, the total amount of the first component is about 0.2% to 2.5% by weight based on the total weight of the hair composition; and the total amount of the second component is about 0.05% to 2% by weight based on the total weight of the hair composition.

14. The hair composition of claim 13, wherein the first component is FiberHance bm and the second component is Restart Pro.

15. The hair composition of claim 12, further comprising at least one additional component selected from the group consisting of surfactants, fatty acid soap, hair and skin conditioning agents, suspending aids, emollients, emulsifiers, rheology modifiers, thickening agents, vitamins, hair growth promoters, self-tanning agents, sunscreens, skin lighteners, anti-aging compounds, anti-wrinkle compounds, hair coloring treatment agents, hair color protecting agents, hair styling products, anti-cellulite compounds, anti-acne compounds, anti-dandruff agents, anti-inflamatory compounds, analgesics, antiperspirant agents, deodorant agents, hair fixatives, particulates, abrasives, moisturizers, antioxidants, keratolytic agents, anti-static agents, foam boosters, hydrotropes, solubilizing agents, chelating agents, antimicrobial agents, antifungal agents, pH adjusting agents, buffering agents, botanicals, hair colorants, oxidizing agents, reducing agents, hair and skin bleaching agents, pigments, anticaries, anti-tartar agents, anti-plaque agents, solvents, preservatives, humectants, exfoliators, natural extracts, alcohols, water, and combinations thereof.

16. The hair composition of claim 2, wherein the hair composition is a shampoo, conditioner, mask, or leave-in treatment.

17. A method of preparing the hair composition of claim 5, comprising combining the components to thereby obtain the hair composition.

18. A method for treating hair or strengthening a hair fiber comprising applying to the hair or the hair fiber the hair composition of claim 1, thereby treating the hair or strengthening the hair fiber, wherein the hair or hair fiber is a hair type selected from straight, wavy, curly, coily, or a combination thereof.

19. A method of reducing and/or preventing breakage of hair comprising applying to the hair the hair composition of claim 1, thereby reducing and/or preventing breakage of hair.

20. The method of claim 19, wherein the hair is virgin hair, hair treated with a chemical or mechanical hair treatment (e.g., bleached hairs, dyed hairs), or damaged or weakened hair.
